(19) United States
(12) Patent Application Publication
(10) Pub. No.: US 2024/0301328 A1
(43) Pub. Date: Sep. 12, 2024

(54) POLYPEPTIDE VARIANTS

(71) Applicant: Novozymes A/S, Bagsvaerd (DK)

(72) Inventors: Jesper Vind, Vaerloese (DK); Trine Holst Soerensen, Copenhagen (DK); Pengfei Tian, Copenhagen (DK); Jesper Henrik Rung, Frederiksberg (DK); Anne Marie Schoenfeld, Basel (CH)

(73) Assignee: Novozymes A/S, Bagsvaerd (DK)

(21) Appl. No.: 18/547,655

(22) PCT Filed: Mar. 9, 2022
(86) PCT No.: PCT/EP2022/056064

§ 371 Date: Aug. 23, 2023

(30) Foreign Application Priority Data
Mar. 12, 2021 (EP) 21162234.5

(51) Int. Cl. C11D 3/386; C12N 9/22; C12R 1/69

(52) U.S. Cl. CPC C11D 3/38636; C12N 9/22; C12R 2001/69

(57) ABSTRACT

The present invention relates to polypeptide variants having DNase activity and detergent compositions comprising the polypeptide variants, as well as methods for obtaining the variants, polynucleotides encoding the variants; nucleic acid constructs, vectors, and host cells comprising the polynucleotides, and methods of using the variants.

### REFERENCE TO A SEQUENCE LISTING

This application contains a Sequence Listing in computer readable form, which is incorporated herein by reference.

### FIELD OF THE INVENTION

The present invention relates to novel DNase variants exhibiting alterations relative to a reference DNase e.g. the parent DNase in one or more properties such as: detergent stability (e.g. improved stability in a detergent composition and/or storage stability in a detergent composition). The present invention also relates to detergent compositions comprising the DNase variants, isolated DNA sequences encoding the variants, expression vectors, host cells, and methods for producing and using the DNase variants of the present invention. The variants of the present invention are suitable for use in cleaning processes and detergent compositions, such as laundry compositions and dishwashing compositions, including hand wash and automatic dishwashing compositions.

### BACKGROUND OF THE INVENTION

Microorganisms generally live attached to surfaces in many natural, industrial and medical environments, encapsulated by extracellular substances including biopolymers and macromolecules. The resulting layer of slime-encapsulated microorganisms is termed a biofilm. Biofilms are the predominant mode of growth of bacteria in the natural environment, and bacteria growing in biofilms exhibit distinct physiological properties. Compared to their planktonically grown counterparts, the bacteria in a biofilm are more resistant to antibiotics, UV irradiation, detergents and the host immune response.

A biofilm may include one or more microorganisms, including gram-positive and gram-negative bacteria, algae, protozoa, and/or yeast or filamentous fungi and viruses and/or bacteriophage. Examples of problematic biofilms are dental plaque, infections on medical implants, but also the initial fouling on ship hulls. Biofilms are attributed to the pathogenesis of many infections in humans and are a significant problem in industry in terms of biofouling of exposed surfaces, where biofilm colonisation can form the base component of a localised ecosystem which can disrupt and interfere with industrial processes and components.

When laundry items like T-shirts or sportswear are used, they are exposed to bacteria from the body of the user and from the rest of the environment in which they are used. Some of these bacteria are capable of adhering to the laundry item and form a biofilm on the item. The presence of bacteria implies that the laundry items become sticky and therefore soil adheres to the sticky areas. This soil has been shown to be difficult to remove by commercially available detergent compositions. Further, when very dirty laundry items are washed together with less dirty laundry items the dirt present in the wash liquor tends to stick to the biofilm. As a result, the laundry item may be more “soiled” after wash than before wash. Further, these bacteria are a source of malodor, which develops after use of the laundry item. The malodor is difficult to remove and may remain even after wash. The reason for this malodor is adhesion of bacteria to the textile surface. Because of the adhesion to the textile, the bacteria may remain even after wash and may continue to be a source of malodor.

International patent application WO 2014/087011 (Novozymes A/S) concerns bacterial deoxyribonuclease (DNase) compounds and methods for biofilm disruption and prevention, and WO 2017/064269 (Novozymes A/S) concerns DNase variants with improved stability. To further improve applicability and/or performance of such enzymes and/or to reduce cost, there is an ongoing search for DNase variants with altered properties, such as increased activity or stability, e.g. improved stability in a detergent composition.

### SUMMARY OF THE INVENTION

The present invention relates to a DNase variant which are particularly useful in detergents and cleaning processes, such as laundry and dishwashing.

In some aspects, the present invention relates to a DNase variant which comprises at least two alterations at positions selected from the group consisting of positions 20, 26, 30, 32, 36, 37, 43, 46, 55, 68, 69, 76, 79, 82, 84, 86, 91, 92, 95, 97, 101, 102, 103, 105, 106, 107, 112, 125, 129, 133, 134, 138, 140, 141, 144, 146, 147, 148, 149, 150, 153, 154, 155, 157, 158, 159, 160, 170, 172, 185, 187, 191, 192, 197, 199, 208, 211, 213, 214, 217 and 218 of SEQ ID NO: 1; for example at least two alterations at positions selected from the group consisting of positions 26, 32, 36, 37, 43, 46, 55, 68, 69, 76, 79, 82, 84, 86, 91, 92, 95, 97, 101, 102, 105, 107, 112, 129, 133, 138, 140, 141, 144, 146, 147, 148, 149, 150, 153, 154, 155, 157, 158, 159, 160, 170, 172, 185, 187, 191, 192, 197, 199, 208, 211, 213, 214, 217 and 218 of SEQ ID NO: 1; wherein the variant has a sequence identity of at least 80%, but less than 100%, to the polypeptide of SEQ ID NO: 1, SEQ ID NO: 2 or SEQ ID NO: 3, and wherein the variant has DNase activity. Preferably, the variant has at least one improved property, in particular improved stability and/or activity, as compared to SEQ ID NO: 1 or a reference DNase without said alterations.

In some aspects, the present invention relates to a DNase variant which comprises at least two alterations selected from the group consisting of G20C, S26*, K30C, D32Q, K36C,H, G37C,R, F43W, D46G, A55I, N68D, S69V, A76I, K82S,T, P84D, T, K86G,L,N,Q,T,V,Y, A91R, L92E, K95I, P97E,N, A101E,N, Q102E, D103S, K105N,G,Q,T,D, S106C,D, F112Y,W, A125C, L129K, N133Q,R, S134C, V138C, N140H, G141Q,R, S144E, N146A, K147N,E,R, V148I, A149D,E,F, Q150D, P153D,V, S154E, K155E,F,L,S,T, Q157D,E, Q158D, T159Q, K160D, T170Q, A172D,E,H,R, K185*, V187N,Y, N191*, K192A, D197K,S, G199Q, Q208V, E211Y,T,P, N213S, N214D, N217A and Y218D, E, wherein positions correspond to the positions of SEQ ID NO: 1; for example at least two alterations selected from the group consisting of S26*, D32Q, K36C, H, G37R, F43W, D46G, A55I, N68D, S69V, A76I, K82S,T, P84D,T, K86G,L,N,Q,T,V,Y, A91R, L92E, K95I, P97E,N, A101E, Q102E, K105N,G,Q,T,D, F112Y,W, L129K, N133Q, V138C, N140H, G141Q,R, S144E, N146A, K147N,E, V148I, A149D,E,F, Q150D, P153D,V, S154E, K155E,F,L,S,T, Q157D,E, Q158D, T159Q, K160D, T170Q, A172D,E,H,R, K185*, V187N,Y, N191*, K192A, D197K,S, G199Q, Q208V, E211Y,T,P, N213S, N214D, N217A and Y218D,E, wherein the positions correspond to the positions of SEQ ID NO: 1; wherein the variant has a sequence identity of at least 80%, but less than 100%, to the polypeptide of SEQ ID NO: 1, SEQ ID NO: 2 or SEQ ID NO: 3, and wherein the variant has DNase activity. Preferably, the variant has at least one improved property, in particular improved stability and/or activity, as compared to SEQ ID NO: 1 or a reference DNase without said alterations.

In some aspects, the present invention relates to a DNase variant which comprises at least two alterations selected from the group consisting of S26*, G37R, S69V, A76I, K86G,L,N,Q,T,V,Y, K95I, A101E, Q102E, K105N,G,Q,T,D, F112Y,W, L129K, N140H, G141Q,R, S144E, N146A, K147N, E, V148I, A149D,E,F, Q150D, P153D,V, S154E, K155E,F,L,S,T, Q157E, Q158D, T159Q, K160D, A172D,E,H,R, V187N,Y, K192A, D197K,S, G199Q, Q208V, E211Y,T,P, N214D, N217A and Y218D, E, wherein the positions correspond to the positions of SEQ ID NO: 1, wherein the variant has a sequence identity of at least 80%, but less than 100%, to the polypeptide of SEQ ID NO: 1, SEQ ID NO: 2 or SEQ ID NO: 3, and wherein the variant has DNase activity. Preferably, the variant has at least one improved property, in particular improved stability and/or activity, as compared to SEQ ID NO: 1 or a reference DNase without said alterations.

In some aspects, the present invention relates to a DNase variant which comprises at least two alterations selected from the group consisting of S26*, G37R, S69V, A76I, K86G,L,N,Q,T,V,Y, K95I, A101E, Q102E, K105N,G,Q,T,D, L129K, G141Q,R, V148I, P153D,V, K155E,F,L,S,T, Q157E, T159Q, A172E, V187N, Y, D197K,S, N214D and Y218D,E, wherein the positions correspond to the positions of SEQ ID NO: 1, wherein the variant has a sequence identity of at least 80%, but less than 100%, to the polypeptide of SEQ ID NO: 1, SEQ ID NO: 2 or SEQ ID NO: 3, and wherein the variant has DNase activity. Preferably, the variant has at least one improved property, in particular improved stability and/or activity, as compared to SEQ ID NO: 1 or a reference DNase without said alterations.

In some aspects, the present invention relates to a DNase variant which has a sequence identity of at least 85%, at least 90%, at least 95%, at least 96%, at least 97%, at least 98% or at least 99% to the polypeptide of SEQ ID NO: 1, SEQ ID NO: 2 or SEQ ID NO: 3.

In some aspects, the present invention relates to a DNase variant wherein the total number of alterations of the DNase variant is 2-20, e.g. 3-15 or 4-12, such as 3, 4, 5, 6, 7, 8, 9, 10 or 11 alterations, compared to SEQ ID NO: 1.

In some preferred embodiments, the DNase variant of the present invention comprises at least one of the following sets of alterations:

| | |
|---|---|
| 1 | N133Q, V148I, L180S |
| 2 | D46G, N133Q, K185* |
| 3 | K86L, K155L, A172H |
| 4 | K86T, K155S, E211T |
| 5 | K86T, K155E, A172R |
| 6 | K86L, T170Q, V187Y |
| 7 | K36H, K86T, K155E |
| 8 | A91R, A149E, K155T |
| 9 | K86Y, Q102E, A149E |
| 10 | A40H, K155S, V187N |
| 11 | K105Q, V187N, Y209H |
| 12 | A55I, K86V, K155T |
| 13 | S144E, A149E, K155E, K192A |
| 14 | K105D, N214D, Y218E |
| 15 | K147E, A172E, V187N, K192A |
| 16 | S69V, Q102E, T159Q |
| 17 | K36C, V138C |
| 18 | L92E, K147N, Q150D, Q157D |
| 19 | S69V, K86T, L92E, V187N |
| 20 | K82S, K155F, N214D |
| 21 | N133Q, N146A, P153D, D197S |
| 22 | S69V, G79C, P107C |
| 23 | K105G, F112Y, A172E |
| 24 | K86Q, N140H, K147E, K155F |
| 25 | F112W, N214D, Y218D |
| 26 | K105N, N140H, K147E, S154E |
| 27 | Q102E, N214D, Y218D |
| 28 | D32Q, K86V, K155E |
| 29 | F112W, N214D, Y218E |
| 30 | K86L, K95I, A149E |
| 31 | K86L, A101E, E211Y |
| 32 | A101E, N214D, Y218D |
| 33 | A101E, N214D, Y218E |
| 34 | A149E, N214D, Y218D |
| 35 | A149E, N214D, Y218E |
| 36 | A149E, P153D, A172E, Q208V |
| 37 | D46G, A101E, G141Q |
| 38 | F43W, K155F, N217A |
| 39 | K105G, N214D, Y218E |
| 40 | K105N, A149D, N213S |
| 41 | K105N, A172R, V187N |
| 42 | K105N, K155F, E211P |
| 43 | K105N, K155L, D197K |
| 44 | K105Q, K155F, N191* |
| 45 | K105Q, K155T, E211T |
| 46 | K105Q, V187N, K192A, D197S |
| 47 | K105T, K155S, V187N |
| 48 | K147N, A172D, V187N, K192A |
| 49 | K147N, Q150D, S154E, D197S |
| 50 | K155E, D197S, N217A |
| 51 | K36C, K105G, V138C |
| 52 | K82T, N140H, K155E |
| 53 | K86G, A101E, V187Y |
| 54 | K86G, K105Q, K155S |
| 55 | K86N, N146A, Q150D, S154E |
| 56 | K86Q, A101E, K105Q, N146A |
| 57 | K86Q, A149E, K155E, A172D |
| 58 | K86T, A101E, G199Q |
| 59 | K86V, A149F, K155F, A172E |
| 60 | K86V, K95I, K155E |
| 61 | K86V, K95I, K155L |
| 62 | K95I, A172D, V187N, K192A |
| 63 | K95I, S144E, A149F, S154E |
| 64 | N68D, N140H, K147N, K155E |
| 65 | P84D, K155L, K160D, A172D |
| 66 | P84D, N140H, K147E, Q158D |
| 67 | P84T, A101E, F112Y |
| 68 | P97E, N140H, A149E, K155L |
| 69 | Q102E, N146A, S154E, K160D |
| 70 | Q102E, N214D, Y218E |
| 71 | S144E, A149F, K155L, D197S |
| 72 | S26*, K105N, G141Q |
| 73 | S69V, K86Q, L92E, K192A |
| 74 | V148I, P153D, A172E, K192A |
| 75 | A101E, A172E, N214D, Y218E |
| 76 | A101E, K105N, N214D, Y218E |
| 77 | S69V, Q102E, P153V, T159Q |
| 78 | S69V, Q102E, Q157E, T159Q |

The present invention also relates to isolated polynucleotides encoding said DNase variants; nucleic acid constructs or vectors, as well as host cells comprising said polynucleotides; methods of producing or obtaining said DNase variants; compositions (e.g. detergent compositions) comprising said DNase variants; as well as use of such DNase variants or compositions.

### OVERVIEW OF SEQUENCE LISTING

SEQ ID NO: 1 mature DNase polypeptide obtained from Aspergillus oryzae
SEQ ID NO: 2 mature DNase polypeptide obtained from Aspergillus oryzae
SEQ ID NO: 3 mature DNase polypeptide obtained from Aspergillus oryzae

### Definitions

DNase: The term “DNase” means a polypeptide with DNase (deoxyribonuclease) activity that catalyzes the hydrolytic cleavage of phosphodiester linkages in DNA, thus degrading DNA. DNases belong to the esterases (EC-number 3.1), a subgroup of the hydrolases. The DNases are classified EC 3.1.21.1. For purposes of the present invention, DNase activity is determined according to the procedure described in the Assay I. The terms “DNase” and “a polypeptide with DNase activity” may be used interchangeably throughout the application.

Parent: The term “parent” means any polypeptide with DNase activity to which an alteration is made to produce the DNase variants of the present invention. Thus a DNase parent or precursor DNase means a DNase in which an alteration is made to produce the DNase variants of the present invention. The terms parent and precursor may be used interchangeably in the present application. Thus, the parent is a DNase having the identical amino acid sequence of the variant but not having the alterations at one or more of the specified positions. It will be understood, that in the present context the expression “having identical amino acid sequence” relates to 100% sequence identity. In a particular embodiment the DNase parent is a DNase with at least 60%, at least 61%, at least 62%, at least 63%, at least 64%, at least 65%, at least 66%, at least 67%, at least 68%, at least 69%, at least 70%, at least 72%, at least 73%, at least 74%, at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99%, at least 99.5% or 100% identity to a polypeptide with SEQ ID NO: 1, SEQ ID NO: 2 or SEQ ID NO: 3.

DNase variant: The term “DNase variant” means a DNase having DNase activity and which comprises an alteration, i.e., a substitution, insertion, and/or deletion at one or more (or one or several) positions compared to its parent e.g. compared to SEQ ID NO: 1. A substitution means a replacement of an amino acid occupying a position with a different amino acid; a deletion means removal of an amino acid occupying a position; and an insertion means adding amino acids e.g. 1 to 10 amino acids, preferably 1-3 amino acids adjacent to an amino acid occupying a position. Preferably, the variant is modified by the hand of man. In one aspect, the variant is at least 1% pure, e.g., at least 5% pure, at least 10% pure, at least 20% pure, at least 40% pure, at least 60% pure, at least 80% pure, and at least 90% pure, as determined by SDS PAGE.

The DNase variants of the present invention preferably have at least one improved property compared to the parent DNase.

Preferably, the DNase variants have improved stability, e.g. improved storage stability in a detergent composition, compared to the parent DNase or the DNase of SEQ ID NO: 1. Storage stability may e.g. be measured and expressed as residual activity (RA), for example by measuring activity of a variant after storage in a liquid detergent compared to the activity of the same variant stored in buffer under the same conditions. The DNase variants of the invention preferably have a residual activity after storage in a liquid detergent, determined e.g. as described in Example 2 herein compared to storage in buffer, for example for 30 min. at room temperature, of at least 0.2, more preferably at least 0.3, such as at least 0.4, at least 0.5, at least 0.6, at least 0.7, at least 0.8 or at least 0.9, and most preferably 1.

In another embodiment, the DNase variants of the invention may have an improved specific activity compared to the parent DNase, e.g. the DNase of SEQ ID NO: 1.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Isolated polynucleotide: The term “isolated polynucleotide” means a polynucleotide that is modified by the hand of man. In one aspect, the isolated polynucleotide is at least 1% pure, e.g., at least 5% pure, at least 10% pure, at least 20% pure, at least 40% pure, at least 60% pure, at least 80% pure, at least 90% pure, and at least 95% pure, as determined by agarose electrophoresis. The polynucleotides may be of genomic, cDNA, RNA, semisynthetic, synthetic origin, or any combinations thereof.

Substantially pure variant: The term “substantially pure variant” means a preparation that contains at most 10%, at most 8%, at most 6%, at most 5%, at most 4%, at most 3%, at most 2%, at most 1%, and at most 0.5% by weight of other polypeptide material with which it is natively or recombinantly associated. Preferably, the variant is at least 92% pure, e.g., at least 94% pure, at least 95% pure, at least 96% pure, at least 97% pure, at least 98% pure, at least 99%, at least 99.5% pure, and 100% pure by weight of the total polypeptide material present in the preparation. The variants of the present invention are preferably in a substantially pure form. This can be accomplished, for example, by preparing the variant by well-known recombinant methods or by classical purification methods.

Wild-type DNase: The term “wild-type DNase” means a DNase expressed by a naturally occurring organism, such as a fungal, bacterium, archaea, yeast, plant or animal found in nature.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide corresponds to the amino acid sequence with SEQ ID NO: 1. It is known in the art that a host cell may produce a mixture of two of more different mature polypeptides (i.e., with a different C terminal and/or N terminal amino acid) expressed by the same polynucleotide. It is also known that different host cells process polypeptides differently, and thus, one host cell expressing a polynucleotide may produce a different mature polypeptide (e.g., having a different C terminal and/or N terminal amino acid) as compared to another host cell expressing the same polynucleotide. In some aspects, a mature polypeptide of the invention is truncated at the N-terminal compared to SEQ ID NO: 1, and may e.g. contain up to 206 amino acid residues, corresponding to SEQ ID NO: 2, or up to 204 amino acid residues, corresponding to SEQ ID NO: 3.

Mature polypeptide coding sequence: The term “mature polypeptide coding sequence” means a polynucleotide that encodes a mature polypeptide having DNase activity.

cDNA: The term “cDNA” means a DNA molecule that can be prepared by reverse transcription from a mature, spliced, mRNA molecule obtained from a prokaryotic or eukaryotic cell. A cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of its polypeptide product. The boundaries of the coding sequence are generally determined by an open reading frame, which usually begins with the ATG start codon or alternative start codons such as GTG and TTG and ends with a stop codon such as TAA, TAG, and TGA. The coding sequence may be a DNA, cDNA, synthetic, or recombinant polynucleotide.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present invention.

Operably linked: The term “operably linked” means a configuration in which a control sequence is placed at an appropriate position relative to the coding sequence of a polynucleotide such that the control sequence directs the expression of the coding sequence.

Control sequences: The term “control sequences” means all components necessary for the expression of a polynucleotide encoding a variant of the present invention. Each control sequence may be native or foreign to the polynucleotide encoding the variant or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a variant.

Expression: The term “expression” includes any step involved in the production of the variant including, but not limited to, transcription, post-transcriptional modification, translation, post-translational modification, and secretion.

Expression vector: The term “expression vector” means a linear or circular DNA molecule that comprises a polynucleotide encoding a variant and is operably linked to additional nucleotides that provide for its expression.

Transcription promoter: The term “transcription promoter” is used for a promoter which is a region of DNA that facilitates the transcription of a particular gene. Transcription promoters are typically located near the genes they regulate, on the same strand and upstream (towards the 5′ region of the sense strand).

Transcription terminator: The term “transcription terminator” is used for a section of the genetic sequence that marks the end of gene or operon on genomic DNA for transcription.

Host cell: The term “host cell” means any cell type that is susceptible to transformation, transfection, transduction, and the like with a nucleic acid construct or expression vector comprising a polynucleotide of the present invention. The term “host cell” encompasses any progeny of a parent cell that is not identical to the parent cell due to mutations that occur during replication.

Improved property: The term “improved property” means a characteristic associated with a variant that is improved compared to the parent and/or compared to a DNase with SEQ ID NO: 1, or compared to a DNase having the identical amino acid sequence of said variant but not having the alterations at one or more of said specified positions. Such improved properties include, but are not limited to, stability, such as detergent stability, and wash performance e.g. deep cleaning effect. The term “deep cleaning” refers to disruption or removal of a biofilm or components of a biofilm such as polysaccharides, proteins, DNA, soil or other components present in the biofilm. The DNase variants of the invention preferably have improved stability, in particular improved storage stability in a detergent composition, compared to the parent DNase or a reference DNase such as the DNase of SEQ ID NO: 1. Alternatively or additionally, the DNase variants of the invention may have improved activity, in particular improved specific activity, compared to the parent DNase or a reference DNase such as the DNase of SEQ ID NO: 1.

Biofilm: A biofilm is any group of microorganisms in which cells stick to each other on a surface, such as a textile, dishware or hard surface. These adherent cells are frequently embedded within a self-produced matrix of extracellular polymeric substance (EPS). Biofilm EPS is a polymeric conglomeration generally composed of extracellular DNA, proteins and polysaccharides. Biofilms may form on living or non-living surfaces. The microbial cells growing in a biofilm are physiologically distinct from planktonic cells of the same organism, which, by contrast, are single cells that may float or swim in a liquid medium. Bacteria living in a biofilm usually have significantly different properties from free-floating bacteria of the same species, as the dense and protected environment of the film allows them to cooperate and interact in various ways. One benefit of this environment is increased resistance to detergents and antibiotics, as the dense extracellular matrix and the outer layer of cells protect the interior of the community. On laundry, biofilm producing bacteria can be found among the following species: Acinetobacter sp., Aeromicrobium sp., Brevundimonas sp., Microbacterium sp., Micrococcus luteus, Pseudomonas sp., Staphylococcus epidermidis, and Stenotrophomonas sp.

Improved DNase activity: The term “improved DNase activity” is defined herein as an altered DNase activity, e.g. by increased catalysis of hydrolytic cleavage of phosphodiester linkages in the DNA, the DNase variant displaying an alteration of the activity relative to the activity of the parent DNase, such as compared to a DNase with SEQ ID NO: 1. The improved activity may e.g. be improved specific activity.

Stability: The term “stability” includes storage stability and stability during use, e.g. during a wash process and reflects the stability of the DNase variant according to the invention as a function of time e.g. how much activity is retained when the DNase variant is kept in solution in particular in a detergent solution. The stability is influenced by many factors e.g. pH, temperature, detergent composition e.g. amount of builder, surfactants etc. The DNase stability may be measured as described in Example 2. The term “improved stability” or “increased stability” is defined herein as a variant DNase displaying an increased stability in solutions, relative to the stability of the parent DNase and/or relative to SEQ ID NO: 1. “Improved stability” and “increased stability” includes detergent stability. The term “detergent stability” or “improved detergent stability” may be improved stability of the DNase activity compared to the DNase parent, e.g. the DNase of SEQ ID NO: 1. The DNase stability is measured as described in Example 2.

Improved wash performance: The term “improved wash performance” may be defined as improved deep cleaning effect (e.g. the disruption or removal of a biofilm or components thereof) of a DNase variant according to the invention compared to the DNase parent or the DNase with SEQ ID NO: 1. The DNase variants may also have improved malodor removal. By the term “malodor” is meant an odor which is not desired on clean items. The cleaned item should smell fresh and clean without malodors adhered to the item. One example of malodor is compounds with an unpleasant smell, which may be produced by microorganisms. Another example is unpleasant smells caused by sweat or body odor adhered to an item which has been in contact with a human or animal. Another example of malodor can be the odor from spices which stick to items, for example curry or other spices which smell strongly. One way of measuring the ability of an item to adhere malodor is by using Assay II disclosed herein.

Wash performance may be expressed as a Remission value of the stained swatches. After washing and rinsing the swatches are spread out flat and allowed to air dry at room temperature overnight. All washed swatches are evaluated the day after the wash. Light reflectance evaluations of the swatches are done using a Macbeth Color Eye 7000 reflectance spectrophotometer with very small aperture. The measurements are made without UV in the incident light and remission value at 460 nm was extracted.

Laundering: The term “laundering” relates to both household laundering and industrial laundering and means the process of treating textiles with a solution containing e.g. a cleaning or detergent composition of the present invention. The laundering process can for example be carried out using e.g. a household or an industrial washing machine or can be carried out by hand.

Detergent composition: The term “detergent composition” (or “cleaning composition”) includes unless otherwise indicated granular or powder-form all-purpose or heavy-duty washing agents, especially cleaning detergents; liquid, gel or paste-form all-purpose washing agents, especially the so-called heavy-duty liquid (HDL) types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, including antibacterial hand-wash types, cleaning bars, soap bars, mouthwashes, denture cleaners, car or carpet shampoos, bathroom cleaners; hair shampoos and hair-rinses; shower gels, foam baths; metal cleaners; as well as cleaning auxiliaries such as bleach additives and “stain-stick” or pre-treat types. The terms “detergent composition” and “detergent formulation” are used in reference to mixtures which are intended for use in a wash medium for the cleaning of soiled objects. In some embodiments, the term is used in reference to laundering fabrics and/or garments (e.g., “laundry detergents”). In alternative embodiments, the term refers to other detergents, such as those used to clean dishes, cutlery, etc. (e.g., “dishwashing detergents”). It is not intended that the present invention be limited to any particular detergent formulation or composition. The term “detergent composition” is not intended to be limited to compositions that contain surfactants. It is intended that in addition to the variants according to the invention, the term encompasses detergents that may contain, e.g., surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxido reductases, bluing agents and fluorescent dyes, antioxidants, and solubilizers.

Fabric: The term “fabric” encompasses any textile material. Thus, it is intended that the term encompass garments, as well as fabrics, yarns, fibers, non-woven materials, natural materials, synthetic materials, and any other textile material.

Textile: The term “textile” refers to woven fabrics, as well as staple fibers and filaments suitable for conversion to or use as yarns, woven, knit, and non-woven fabrics. The term encompasses yarns made from natural, as well as synthetic (e.g., manufactured) fibers. The term, “textile materials” is a general term for fibers, yarn intermediates, yarn, fabrics, and products made from fabrics (e.g., garments and other articles).

Non-fabric detergent compositions: The term “non-fabric detergent compositions” include non-textile surface detergent compositions, including but not limited to compositions for hard surface cleaning, such as dishwashing detergent compositions including manual dishwashing compositions, oral detergent compositions, denture detergent compositions, and personal cleansing compositions.

Effective amount of enzyme: The term “effective amount of enzyme” refers to the quantity of enzyme necessary to achieve the enzymatic activity required in the specific application, e.g., in a defined detergent composition. Such effective amounts are readily ascertained by one of ordinary skill in the art and are based on many factors, such as the particular enzyme used, the cleaning application, the specific composition of the detergent composition, and whether a liquid or dry (e.g., granular, bar) composition is required, and the like. The term “effective amount” of a DNase variant refers to the quantity of DNase variant described hereinbefore that achieves a desired level of enzymatic activity, e.g., in a defined detergent composition.

Relevant washing conditions: The term “relevant washing conditions” is used herein to indicate the conditions, particularly washing temperature, time, washing mechanics, detergent concentration, type of detergent and water hardness, actually used in households in a detergent market segment.

Wash liquor: The term “wash liquor” refers to an aqueous solution comprising a DNase variant of the invention. A wash liquor is a solution, e.g. found in a washing machine or dishwasher, containing water and a detergent composition comprising the DNase. The detergent composition, prior to being mixed with water to form a wash liquor, may be in any form as described elsewhere herein, for example a liquid or powder.

Water hardness: The term “water hardness” or “degree of hardness” or “dH” or “°dH” as used herein refers to German degrees of hardness. One degree is defined as 10 milligrams of calcium oxide per liter of water.

Adjunct materials: The term “adjunct materials” means any liquid, solid or gaseous material selected for the particular type of detergent composition desired and the form of the product (e.g., liquid, granule, powder, bar, paste, spray, tablet, gel, or foam composition), which materials are also preferably compatible with the DNase variant enzyme used in the composition. In some embodiments, granular compositions are in “compact” form, while in other embodiments, the liquid compositions are in a “concentrated” form.

Low detergent concentration: The term “low detergent concentration” system includes detergents where less than about 800 ppm of detergent components is present in the wash water. Asian, e.g., Japanese detergents are typically considered low detergent concentration systems.

Medium detergent concentration: The term “medium detergent concentration” system includes detergents wherein between about 800 ppm and about 2000 ppm of detergent components is present in the wash water. North American detergents are generally considered to be medium detergent concentration systems.

High detergent concentration: The term “high detergent concentration” system includes detergents wherein greater than about 2000 ppm of detergent components is present in the wash water. European detergents are generally considered to be high detergent concentration systems.

Sequence identity: Sequence identity percentages may be referred to herein with reference to SEQ ID NO: 1, SEQ ID NO: 2 or SEQ ID NO: 3. As explained above, it is known that a host cell may produce a mixture of two of more different mature polypeptides, and that different host cells process polypeptides differently. In the case of the mature polypeptide of SEQ ID NO: 1, it has been found that this may be expressed with different N-terminal truncations, for example as the polypeptide of SEQ ID NO: 2 (206 amino acid residues, corresponding to amino acids 16 to 221 of SEQ ID NO: 1) or SEQ ID NO: 3 (204 amino acid residues, corresponding to amino acids 18 to 221 of SEQ ID NO: 1). Any reference herein to a polypeptide having a given percent sequence identity to SEQ ID NO: 1 should therefore be understood as including polypeptides having the same percent sequence identity to SEQ ID NO: 2 or to SEQ ID NO: 3.

### Conventions for Designation of Variants

For purposes of the present invention, the mature polypeptide disclosed in SEQ ID NO: 1 is used to determine the corresponding amino acid residue in another DNase. The amino acid sequence of another DNase is aligned with the polypeptide disclosed in SEQ ID NO: 1, and based on the alignment, the amino acid position number corresponding to any amino acid residue in the polypeptide disclosed in SEQ ID NO: 1 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another DNase can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

When the other enzyme has diverged from the polypeptide of SEQ ID NO: 1 such that traditional sequence-based comparison fails to detect their relationship (Lindahl and Elofsson, 2000, J. Mol. Biol. 295: 613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example, the SCOP super families of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g., Holm and Park, 2000, Bioinformatics 16: 566-567).

In describing the variants of the present invention, the nomenclature described below is adapted for ease of reference. The accepted IUPAC single letter or three letter amino acid abbreviation is employed.

Substitutions. For an amino acid substitution, the following nomenclature is used: Original amino acid, position, substituted amino acid. Accordingly, the substitution of threonine at position 226 with alanine is designated as “T226A” (or “Thr226Ala” using the three-letter abbreviation). Multiple mutations may be separated by addition marks (“+”), e.g., “G205R+S411F”, or by a comma, e.g. “G205R, S411F”, representing substitutions at positions 205 and 411 of glycine (G) with arginine (R) and serine (S) with phenylalanine (F), respectively.

Deletions. For an amino acid deletion, the following nomenclature is used: Original amino acid, position, *. Accordingly, the deletion of glycine at position 195 is designated as “G195*”. Multiple deletions may be separated by addition marks (“+”), e.g., “G195*+S411*” or by a comma, e.g. “G195*, S411*”.

Insertions. For an amino acid insertion, the following nomenclature is used: Original amino acid, position, original amino acid, inserted amino acid. Accordingly the insertion of lysine after glycine at position 195 is designated “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “G195GKA”. An indication of an insertion at a particular position is understood as being an insertion after the original amino acid residue. For example, an “insertion at position 195” is understood to be an insertion after the original residue in position 195.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G-K-A |

Multiple alterations. Variants comprising multiple alterations are separated by addition marks (“+”) or commas, e.g., “R170Y+G195E” or “R170Y,G195E” representing a substitution of arginine and glycine at positions 170 and 195 with tyrosine and glutamic acid, respectively.

Different alterations. Where different alterations can be introduced at a position, the different alterations may be separated by a comma, e.g., “R170Y,E” represents a substitution of arginine at position 170 with tyrosine or glutamic acid. Thus, “Y167G,A+R170G,A” designates the following variants: “Y167G+R170G”, “Y167G+R170A”, “Y167A+R170G”, and “Y167A+R170A”.

Alternatively, different alterations may be indicated using brackets, e.g., R170 [Y,G].

### DETAILED DESCRIPTION OF THE INVENTION

The present invention provides novel DNases obtained from Aspergillus, in particular, Aspergillus oryzae. The DNases of the present invention comprise at least 80% sequence identity to a polypeptide of SEQ ID NO: 1, SEQ ID NO: 2 or SEQ ID NO: 3, and comprise at least two alterations made at positions equivalent to positions in SEQ ID NO: 1.

The present invention also relates to methods for generating DNase variants.

One embodiment of the present invention relates to DNase variants having at least 80% identity to SEQ ID NO: 1 and comprising at least two alterations at positions selected from the group consisting of positions 26, 32, 36, 37, 43, 46, 55, 68, 69, 76, 79, 82, 84, 86, 91, 92, 95, 97, 101, 102, 105, 107, 112, 129, 133, 138, 140, 141, 144, 146, 147, 148, 149, 150, 153, 154, 155, 157, 158, 159, 160, 170, 172, 185, 187, 191, 192, 197, 199, 208, 211, 213, 214, 217 and 218 of SEQ ID NO: 1, wherein the variant has DNase activity, and wherein the variant has improved stability and/or activity as compared to SEQ ID NO: 1 or a reference DNase. Each alteration is independently a substitution, insertion or deletion.

One embodiment of the present invention relates to a DNase variant comprising at least two alterations, e.g., at least three, at least four, at least five, at least six, at least seven, at least eight, at least nine, or at least ten alterations, selected from the group consisting of S26*, D32Q, K36C, H, G37R, F43W, D46G, A55I, N68D, S69V, A76I, K82S,T, P84D, T, K86G,L,N,Q,T,V,Y, A91R, L92E, K95I, P97E,N, A101E, Q102E, K105N,G,Q,T,D, F112Y,W, L129K, N133Q, V138C, N140H, G141Q,R, S144E, N146A, K147N,E, V148I, A149D,E,F, Q150D, P153D,V, S154E, K155E,F,L,S,T, Q157D,E, Q158D, T159Q, K160D, T170Q, A172D,E,H,R, K185*, V187N,Y, N191*, K192A, D197K,S, G199Q, Q208V, E211Y,T,P, N213S, N214D, N217A and Y218D,E, wherein the variant has a sequence identity of at least 80%, but less than 100%, to the polypeptide of SEQ ID NO: 1, SEQ ID NO: 2 or SEQ ID NO: 3; wherein the variant has DNase activity, and wherein the variant has improved stability and/or activity as compared to SEQ ID NO: 1 or a reference DNase, and wherein the positions correspond to the positions of SEQ ID NO: 1.

A preferred embodiment of the present invention relates to a DNase variant comprising at least two alterations selected from the group consisting of S26*, G37R, S69V, A76I, K86G,L,N,Q,T,V,Y, K95I, A101E, Q102E, K105N,G,Q,T,D, F112Y,W, L129K, N140H, G141Q,R, S144E, N146A, K147N,E, V148I, A149D,E,F, Q150D, P153D,V, S154E, K155E,F,L,S,T, Q157E, Q158D, T159Q, K160D, A172D,E,H,R, V187N,Y, K192A, D197K,S, G199Q, Q208V, E211Y,T,P, N214D, N217A and Y218D, E, wherein the variant has a sequence identity of at least 80%, but less than 100%, to the polypeptide of SEQ ID NO: 1, SEQ ID NO: 2 or SEQ ID NO: 3; wherein the variant has DNase activity, and wherein the variant has improved stability and/or activity as compared to SEQ ID NO: 1 or a reference DNase, and wherein the positions correspond to the positions of SEQ ID NO: 1. The group of mutations are located at 36 different positions. Thus, in an embodiment, the variants of the present invention comprise at least two alterations, e.g., at least three, at least four, at least five, at least six, at least seven, at least eight, at least nine, or at least ten, etc.

Another preferred embodiment of the present invention relates to a DNase variant comprising at least two alterations selected from the group consisting of S26*, G37R, S69V, A76I, K86G,L,N,Q,T,V,Y, K95I, A101E, Q102E, K105N,G,Q,T,D, L129K, G141Q,R, V148I, P153D,V, K155E,F,L,S,T, Q157E, T159Q, A172E, V187N, Y, D197K,S, N214D and Y218D,E, wherein the variant has a sequence identity of at least 80%, but less than 100%, to the polypeptide of SEQ ID NO: 1, SEQ ID NO: 2 or SEQ ID NO: 3; wherein the variant has DNase activity, and wherein the variant has improved stability and/or activity as compared to SEQ ID NO: 1 or a reference DNase, and wherein the positions correspond to the positions of SEQ ID NO: 1. The group of mutations are located at 21 different positions. Thus, in an embodiment, the variants of the present invention comprise at least two alterations, e.g., at least three, at least four, at least five, at least six, at least seven, at least eight, at least nine, or at least ten, etc.

In some embodiments, the DNase variant has a sequence identity of at least 85%, e.g., at least 90%, at least 95%, at least 96%, at least 97%, at least 98% or at least 99%, but less than 100%, to the amino acid sequence of the parent DNase.

In some embodiments, the DNase variant has a sequence identity of at least 85%, e.g., at least 90%, at least 95%, at least 96%, at least 97%, at least 98% or at least 99%, but less than 100%, to the polypeptide shown in SEQ ID NO: 1, or to SEQ ID NO: 2 or SEQ ID NO: 3.

The percent sequence identity is determined using the Needleman-Wunsch algorithm (Needleman and Wunsch, 1970, J. Mol. Biol. 48: 443-453) as described in “Conventions for Designation of Variants”.

In one embodiment, the alteration is a substitution. In another embodiment, the alteration is a deletion. In one aspect, the number of alterations in the variants of the present invention is 2-20, e.g. 3-15 or 4-12, such as 3, 4, 5, 6, 7, 8, 9, 10 or 11 alterations.

Exemplary descriptions of preferred alterations are provided below.

### K36H,C

In one aspect, the variants comprise the substitution K36H or K36C of SEQ ID NO: 1. The substitution confers improved detergent stability to the variants.

In a preferred embodiment, the variant comprises SEQ ID NO: 1 with the substitution K36H or K36C, and at least one substitution, e.g., at least two, at least three, at least four, or at least five substitutions, selected from the group consisting of G37R, A76I, L129K, K86T, K155E, K105G, V138C and G141R; and wherein the variant has DNase activity; and wherein position numbers are based on the numbering of SEQ ID NO: 1. In a particularly preferred embodiment, the variant comprises SEQ ID NO: 1 with the substitution K36H or K36C, and at least one substitution, e.g., at least two, at least three, or at least four substitutions, selected from the group consisting of K86T, K155E, K105G and V138C, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
K36H+K86T+K155E,
K36C+V138C, and
K36C+K105G+V138C;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

### N68D

In one aspect, the variants comprise the substitution N68D of SEQ ID NO: 1. The substitution confers improved detergent stability to the variants.

In a preferred embodiment, the variant comprises SEQ ID NO: 1 with the substitution N68D, and at least one substitution, e.g., at least two, at least three, at least four, at least five or at least six substitutions, selected from the group consisting of G37R, A76I, L129K, N140H, G141R, K147N and K155E; and wherein the variant has DNase activity; and wherein position numbers are based on the numbering of SEQ ID NO: 1. In a particularly preferred embodiment, the variant comprises SEQ ID NO: 1 with the substitution N68D, and at least one substitution, e.g., at least two or at least three substitutions, selected from the group consisting of N140H, K147N and K155E, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90% at least 95%, at least 96%, at least 97% or at least 98%, but less than 100%, to the parent polypeptide of SEQ ID NO: 1, SEQ ID NO: 2 or SEQ ID NO: 3; wherein the variant comprises the following set of alterations: N68D+N140H+K147N+K155E; and wherein the variant having the above set of alterations may further comprise at least one additional substitution disclosed herein, e.g. at least one substitution selected from the group consisting of G37R, A76I, L129K and G141R; wherein the variant has DNase activity; and wherein position numbers are based on the numbering of SEQ ID NO: 1.

### S69V

In one aspect, the variants comprise the substitution S69V of SEQ ID NO: 1. The substitution confers improved detergent stability to the variants.

In a preferred embodiment, the variant comprises SEQ ID NO: 1 with the substitution S69V, and at least one substitution, e.g., at least two, at least three, at least four, at least five or at least six substitutions, selected from the group consisting of G37R, A76I, K86Q, T, L92E, Q102E, L129K, G141R, P153V, Q157E, T159Q, V187N and K192A; and wherein the variant has DNase activity; and wherein position numbers are based on the numbering of SEQ ID NO: 1. In a particularly preferred embodiment, the variant comprises SEQ ID NO: 1 with the substitution S69V, and at least one substitution, e.g., at least two, at least three, or at least four substitutions, selected from the group consisting of K86Q, T, L92E, Q102E, P153V, Q157E, T159Q, V187N and K192A, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. In some preferred embodiments, variants of the invention comprise the substitutions S69V+Q102E, and optionally at least one additional substitution disclosed herein. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
S69V+Q102E;
S69V+K86Q+L92E+K192A,
S69V+G79C+P107C,
S69V+K86T+L92E+V187N,
S69V+Q102E+T159Q,
S69V+Q102E+Q157E+T159Q, and
S69V+Q102E+P153V+T159Q;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

### K82S

In one aspect, the variants comprise the substitution K82S of SEQ ID NO: 1. The substitution confers improved detergent stability to the variants.

In a preferred embodiment, the variant comprises SEQ ID NO: 1 with the substitution K82S, and at least one substitution, e.g., at least two, at least three, at least four, at least five or at least six substitutions, selected from the group consisting of G37R, A76I, L129K, G141R, K155F and N214D; and wherein the variant has DNase activity; and wherein position numbers are based on the numbering of SEQ ID NO: 1. In a particularly preferred embodiment, the variant comprises SEQ ID NO: 1 with the substitution K82S, and at least one substitution, e.g., two substitutions, selected from the group consisting of K155F and N214D, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises the following set of alterations: K82S+K155F+N214D; and wherein the variant having the above set of alterations may further comprise at least one additional substitution disclosed herein, e.g. at least one substitution selected from the group consisting of G37R, A76I, L129K and G141R; wherein the variant has DNase activity; and wherein position numbers are based on the numbering of SEQ ID NO: 1.

### P84D,T

In one aspect, the variants comprise the substitution P84D or P84T of SEQ ID NO: 1. The substitution confers improved detergent stability to the variants.

In a preferred embodiment, the variant comprises SEQ ID NO: 1 with the substitution P84D or P84T, and at least one substitution, e.g., at least two, at least three, at least four, at least five or at least six substitutions, selected from the group consisting of G37R, A76I, A101E, F112Y, L129K, G141R, N140H, K147E, K155L, Q158D, K160D and A172D; and wherein the variant has DNase activity; and wherein position numbers are based on the numbering of SEQ ID NO: 1. In a particularly preferred embodiment, the variant comprises SEQ ID NO: 1 with the substitution P84D or P84T, and at least one substitution, e.g., at least two, at least three, at least four or at least five substitutions, selected from the group consisting of A101E, F112Y, N140H, K147E, K155L, Q158D, K160D and A172D, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
P84D+K155L+K160D+A172D,
P84D+N140H+K147E+Q158D, and
P84T+A101E+F112Y;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

### K86G,L,N,Q,T,V,Y

In one aspect, the variants comprise the substitution K86G,L,N,Q,T,V,Y of SEQ ID NO: 1. The substitution confers improved detergent stability to the variants.

In a preferred embodiment, the variant comprises SEQ ID NO: 1 with the substitution K86G, and at least one substitution, e.g., at least two, at least three, at least four, at least five or at least six substitutions, selected from the group consisting of G37R, A76I, A101E, K105Q, L129K, G141R, K155S and V187N; and wherein the variant has DNase activity; and wherein position numbers are based on the numbering of SEQ ID NO: 1. In a particularly preferred embodiment, the variant comprises SEQ ID NO: 1 with the substitution K86G, and at least one substitution, e.g., at least two, at least three or at least four substitutions, selected from the group consisting of A101E, K105Q, K155S and V187N, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
K86G+K105Q+K155S, and
K86G+A101E+V187Y;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

In a preferred embodiment, the variant comprises SEQ ID NO: 1 with the substitution K86L, and at least one substitution, e.g., at least two, at least three, at least four, at least five or at least six substitutions, selected from the group consisting of G37R, A76I, K95I, A101E, L129K, G141R, A149E, K155L, T170Q, A172H, V187Y and E211Y; and wherein the variant has DNase activity; and wherein position numbers are based on the numbering of SEQ ID NO: 1. In a particularly preferred embodiment, the variant comprises SEQ ID NO: 1 with the substitution K86L, and at least one substitution, e.g., at least two, at least three or at least four substitutions, selected from the group consisting of K95I, A101E, A149E, K155L, T170Q, A172H, V187Y and E211Y, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
K86L+A101E+E211Y,
K86L+K95I+A149E,
K86L+T170Q+V187Y, and
K86L+K155L+A172H;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

In a preferred embodiment, the variant comprises SEQ ID NO: 1 with the substitution K86N, and at least one substitution, e.g., at least two, at least three, at least four, at least five or at least six substitutions, selected from the group consisting of G37R, A76I, L129K, G141R, N146A, Q150D and S154E; and wherein the variant has DNase activity; and wherein position numbers are based on the numbering of SEQ ID NO: 1. In a particularly preferred embodiment, the variant comprises SEQ ID NO: 1 with the substitution K86N, and at least one substitution, e.g., at least two or at least three substitutions, selected from the group consisting of N146A, Q150D and S154E, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises the following set of alterations: K86N+N146A+Q150D+S154E; wherein the variant having the above set of alterations may further comprise at least one additional substitution disclosed herein, e.g. at least one substitution selected from the group consisting of G37R, A76I, L129K and G141R; wherein the variant has DNase activity; and wherein position numbers are based on the numbering of SEQ ID NO: 1.

In a preferred embodiment, the variant comprises SEQ ID NO: 1 with the substitution K86Q, and at least one substitution, e.g., at least two, at least three, at least four, at least five or at least six substitutions, selected from the group consisting of G37R, S69V, A76I, L92E, A101E, K105Q, L129K, N140H, G141R, N146A, K147E, A149E, K155E,F, A172D and K192A; and wherein the variant has DNase activity; and wherein position numbers are based on the numbering of SEQ ID NO: 1. In a particularly preferred embodiment, the variant comprises SEQ ID NO: 1 with the substitution K86Q, and at least one substitution, e.g., at least two, at least three or at least four substitutions, selected from the group consisting of S69V, L92E, A101E, K105Q, N140H, N146A, K147E, A149E, K155E,F, A172D and K192A, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
K86Q+A101E+K105Q+N146A,
S69V+K86Q+L92E+K192A,
K86Q+A149E+K155E+A172D, and
K86Q+N140H+K147E+K155F;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

In a preferred embodiment, the variant comprises SEQ ID NO: 1 with the substitution K86T, and at least one substitution, e.g., at least two, at least three, at least four, at least five or at least six substitutions, selected from the group consisting of K36H, G37R, A76I, S69V, L92E, A101E, L129K, G141R, K155E,S, A172R, V187N, G199Q and E211T; and wherein the variant has DNase activity; and wherein position numbers are based on the numbering of SEQ ID NO: 1. In a particularly preferred embodiment, the variant comprises SEQ ID NO: 1 with the substitution K86T, and at least one substitution, e.g., at least two, at least three or at least four substitutions, selected from the group consisting of K36H, S69V, L92E, A101E, K155E,S, A172R, V187N, G199Q and E211T, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
K86T+A101E+G199Q,
S69V+K86T+L92E+V187N,
K36H+K86T+K155E,
K86T+K155E+A172R, and
K86T+K155S+E211T;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

In a preferred embodiment, the variant comprises SEQ ID NO: 1 with the substitution K86V, and at least one substitution, e.g., at least two, at least three, at least four, at least five or at least six substitutions, selected from the group consisting of D32Q, G37R, A55I, A76I, K95I, L129K, G141R, A149F, K155E,F,L,T, and A172E; and wherein the variant has DNase activity; and wherein position numbers are based on the numbering of SEQ ID NO: 1. In a particularly preferred embodiment, the variant comprises SEQ ID NO: 1 with the substitution K86V, and at least one substitution, e.g., at least two, at least three or at least four substitutions, selected from the group consisting of D32Q, A55I, K95I, A149F, K155E,F,L,T, and A172E, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
K86V+K95I+K155E,
K86V+A149F+K155F+A172E,
K86V+K95I+K155L,
A55I+K86V+K155T, and
D32Q+K86V+K155E;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

In a preferred embodiment, the variant comprises SEQ ID NO: 1 with the substitution K86Y, and at least one substitution, e.g., at least two, at least three, at least four, at least five or at least six substitutions, selected from the group consisting of G37R, A76I, Q102E, L129K, G141R and A149E; and wherein the variant has DNase activity; and wherein position numbers are based on the numbering of SEQ ID NO: 1. In a particularly preferred embodiment, the variant comprises SEQ ID NO: 1 with the substitution K86Y, and at least one substitution, e.g., at least two substitutions, selected from the group consisting of Q102E and A149E, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises the following set of alterations: K86Y+Q102E+A149E; wherein the variant having the above set of alterations may further comprise at least one additional substitution disclosed herein, e.g. at least one substitution selected from the group consisting of G37R, A76I, L129K and G141R; wherein the variant has DNase activity; and wherein position numbers are based on the numbering of SEQ ID NO: 1.

### K95I

In one aspect, the variants comprise the substitution K95I of SEQ ID NO: 1. The substitution confers improved detergent stability to the variants.

In a preferred embodiment, the variant comprises SEQ ID NO: 1 with the substitution K95I, and at least one substitution, e.g., at least two, at least three, at least four, at least five, or at least six substitutions, selected from the group consisting of G37R, A76I, K86V,L, L129K, G141R, S144E, A149E,F, S154E, K155E,L, A172D, V187N and K192A; and wherein the variant has DNase activity; and wherein position numbers are based on the numbering of SEQ ID NO: 1. In a particularly preferred embodiment, the variant comprises SEQ ID NO: 1 with the substitution K95I, and at least one substitution, e.g., at least two, at least three, at least four, or at least five substitutions, selected from the group consisting of K86V,L, S144E, A149E,F, S154E, K155E,L, A172D, V187N and K192A, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
K86V+K95I+K155E,
K95I+A172D+V187N+K192A,
K95I+S144E+A149F+S154E,
K86V+K95I+K155L, and
K86L+K95I+A149E;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

### P97E

In one aspect, the variants comprise the substitution P97E of SEQ ID NO: 1. The substitution confers improved detergent stability to the variants.

In a preferred embodiment, the variant comprises SEQ ID NO: 1 with the substitution P97E, and at least one substitution, e.g., at least two, at least three, at least four, at least five, or at least six substitutions, selected from the group consisting of G37R, A76I, L129K, G141R, N140H, A149E and K155L; and wherein the variant has DNase activity; and wherein position numbers are based on the numbering of SEQ ID NO: 1. In a particularly preferred embodiment, the variant comprises SEQ ID NO: 1 with the substitution P97E, and at least one substitution, e.g., at least two, or at least three substitutions, selected from the group consisting of N140H, A149E and K155L, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises the following set of alterations P97E+N140H+A149E+K155L; and wherein the variant having the above set of alterations may further comprise at least one additional substitution disclosed herein, e.g. at least one substitution selected from the group consisting of G37R, A76I, L129K and G141R; and wherein the variant has DNase activity; and wherein position numbers are based on the numbering of SEQ ID NO: 1.

### A101E

In one aspect, the variants comprise the substitution A101E of SEQ ID NO: 1. The substitution confers improved detergent stability to the variants.

In a preferred embodiment, the variant comprises SEQ ID NO: 1 with the substitution A101E, and at least one substitution, e.g., at least two, at least three, at least four, at least five, or at least six substitutions, selected from the group consisting of G37R, D46G, A76I, P84T, K86T,L,Q,G, K105N,Q, F112Y, L129K, G141R,Q, N146A, A172E, V187Y, G199Q, E211Y, N214D and Y218D, E; and wherein the variant has DNase activity; and wherein position numbers are based on the numbering of SEQ ID NO: 1. In a particularly preferred embodiment, the variant comprises SEQ ID NO: 1 with the substitution A101E, and at least one substitution, e.g., at least two, at least three, at least four, or at least five substitutions, selected from the group consisting of D46G, P84T, K86T,L,Q,G, K105N,Q, F112Y, G141Q, N146A, A172E, V187Y, G199Q, E211Y, N214D and Y218D, E, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
K86Q+A101E+K105Q+N146A,
A101E+N214D+Y218E,
K86G+A101E+V187Y,
P84T+A101E+F112Y,
A101E+N214D+Y218D,
K86T+A101E+G199Q,
D46G+A101E+G141Q,
A101E+A172E+N214D+Y218E,
A101E+K105N+N214D+Y218E, and
K86L+A101E+E211Y;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

### K105N,Q,T

In one aspect, the variants comprise the substitution K105N,Q,T of SEQ ID NO: 1. The substitution confers improved detergent stability to the variants.

In a preferred embodiment, the variant comprises SEQ ID NO: 1 with the substitution K105N, and at least one alterations, e.g., at least two, at least three, at least four, at least five, or at least six alterations, selected from the group consisting of S26*, G37R, A76I, A101E, L129K, N140H, G141R,Q, K147E, A149D, S154E, K155F,L, A172R, V187N, D197K, E211P, N213S, N214D and Y218E; and wherein the variant has DNase activity; and wherein position numbers are based on the numbering of SEQ ID NO: 1. In a particularly preferred embodiment, the variant comprises SEQ ID NO: 1 with the substitution K105N, and at least one alterations, e.g., at least two, at least three, at least four, or at least five alterations, selected from the group consisting of S26*, A101E, N140H, G141Q, K147E, A149D, S154E, K155F,L, A172R, V187N, D197K, E211P, N213S, N214D and Y218E, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
K105N+A149D+N213S,
K105N+K155L+D197K,
K105N+K155F+E211P,
S26*+K105N+G141Q,
K105N+A172R+V187N,
K105N+N140H+K147E+S154E, and
A101E+K105N+N214D+Y218E;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

In a preferred embodiment, the variant comprises SEQ ID NO: 1 with the substitution K105Q, and at least one alteration, e.g., at least two, at least three, at least four, at least five, or at least six alterations, selected from the group consisting of G37R, A76I, Q86G,Q, A101E, L129K, G141R, N146A, K155F,S,T, V187N, N191*, K192A, D197S and E211T; and wherein the variant has DNase activity; and wherein position numbers are based on the numbering of SEQ ID NO: 1. In a particularly preferred embodiment, the variant comprises SEQ ID NO: 1 with the substitution K105Q, and at least one alteration, e.g., at least two, at least three, at least four, or at least five alterations, selected from the group consisting of Q86G,Q, A101E, N146A, K155F,S,T, V187N, N191*, K192A, D197S and E211T, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
K86G+K105Q+K155S,
K86Q+A101E+K105Q+N146A,
K105Q+K155F+N191*,
K105Q+V187N+K192A+D197S, and
K105Q+K155T+E211T;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

In a preferred embodiment, the variant comprises SEQ ID NO: 1 with the substitution K105T, and at least one substitution, e.g., at least two, at least three, at least four or at least five substitutions, selected from the group consisting of G37R, A76I, L129K, G141R, K155S and V187N; and wherein the variant has DNase activity; and wherein position numbers are based on the numbering of SEQ ID NO: 1. In a particularly preferred embodiment, the variant comprises SEQ ID NO: 1 with the substitution K105T, and at least one substitution selected from the group consisting of K155S and V187N, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises the following set of alterations: K105T+K155S+V187N; and wherein the variant having the above set of alterations may further comprise at least one additional substitution disclosed herein, e.g. at least one substitution selected from the group consisting of G37R, A76I, L129K and G141R; wherein the variant has DNase activity; and wherein position numbers are based on the numbering of SEQ ID NO: 1.

### N133Q

In one aspect, the variants comprise the substitution N133Q of SEQ ID NO: 1. The substitution confers improved detergent stability to the variants.

In a preferred embodiment, the variant comprises SEQ ID NO: 1 with the substitution N133Q, and at least one alteration, e.g., at least two, at least three, at least four, at least five, or at least six alterations, selected from the group consisting of G37R, D46G A76I, L129K, G141R, N146A, V148I, P153D, L180S, K185* and D197S; and wherein the variant has DNase activity; and wherein position numbers are based on the numbering of SEQ ID NO: 1. In a particularly preferred embodiment, the variant comprises SEQ ID NO: 1 with the substitution N133Q, and at least one alteration, e.g., at least two, at least three, at least four, or at least five substitutions, selected from the group consisting of D46G, N146A, V148I, P153D, L180S, K185* and D197S, and optionally at least one substitution, e.g., at least two, at least three, or at least four alterations, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
N133Q+N146A+P153D+D197S,
D46G+N133Q+K185*, and
N133Q+V148I+L180S;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

### S144E

In one aspect, the variants comprise the substitution S144E of SEQ ID NO: 1. The substitution confers improved detergent stability to the variants.

In a preferred embodiment, the variant comprises SEQ ID NO: 1 with the substitution S144E, and at least one substitution, e.g., at least two, at least three, at least four, at least five, or at least six substitutions, selected from the group consisting of G37R, A76I, K95I, L129K, G141R, A149E,F, K155E,L, S154E, K192A and D197S; and wherein the variant has DNase activity; and wherein position numbers are based on the numbering of SEQ ID NO: 1. In a particularly preferred embodiment, the variant comprises SEQ ID NO: 1 with the substitution S144E, and at least one substitution, e.g., at least two, at least three, or at least four substitutions, selected from the group consisting of K95I, A149E,F, K155E,L, S154E, K192A and D197S, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
S144E+A149F+K155L+D197S,
K95I+S144E+A149F+S154E, and
S144E+A149E+K155E+K192A;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

### N146A

In one aspect, the variants comprise the substitution N146A of SEQ ID NO: 1. The substitution confers improved detergent stability to the variants.

In a preferred embodiment, the variant comprises SEQ ID NO: 1 with the substitution N146A, and at least one substitution, e.g., at least two, at least three, at least four, at least five, or at least six substitutions, selected from the group consisting of G37R, A76I, K86Q, N, A101E, Q102E, K105Q, L129K, N133Q, G141R, N146A, Q150D, P153D, S154E, K160D and D197S; and wherein the variant has DNase activity; and wherein position numbers are based on the numbering of SEQ ID NO: 1. In a particularly preferred embodiment, the variant comprises SEQ ID NO: 1 with the substitution N146A, and at least one substitution, e.g., at least two, at least three, or at least four substitutions, selected from the group consisting of K86Q, N, A101E, Q102E, K105Q, N133Q, N146A, Q150D, P153D, S154E, K160D and D197S, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
K86Q+A101E+K105Q+N146A,
K86N+N146A+Q150D+S154E,
Q102E+N146A+S154E+K160D, and
N133Q+N146A+P153D+D197S;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

### K147N

In one aspect, the variants comprise the substitution K147N of SEQ ID NO: 1. The substitution confers improved detergent stability to the variants.

In a preferred embodiment, the variant comprises SEQ ID NO: 1 with the substitution K147N, and at least one substitution, e.g., at least two, at least three, at least four, at least five, or at least six substitutions, selected from the group consisting of G37R, N68D, A76I, L92E, L129K, N140H, G141R, Q150D, S154E, K155E, Q157D, A172D, V187N, K192A and D197S; and wherein the variant has DNase activity; and wherein position numbers are based on the numbering of SEQ ID NO: 1. In a particularly preferred embodiment, the variant comprises SEQ ID NO: 1 with the substitution K147N, and at least one substitution, e.g., at least two, at least three, or at least four substitutions, selected from the group consisting of N68D, L92E, N140H, Q150D, S154E, K155E, Q157D, A172D, V187N, K192A and D197S, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
N68D+N140H+K147N+K155E,
K147N+Q150D+S154E+D197S,
K147N+A172D+V187N+K192A, and
L92E+K147N+Q150D+Q157D;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

### V148I

In one aspect, the variants comprise the substitution V148I of SEQ ID NO: 1. The substitution confers improved detergent stability to the variants.

In a preferred embodiment, the variant comprises SEQ ID NO: 1 with the substitution V148I, and at least one substitution, e.g., at least two, at least three, at least four, or at least five substitutions, selected from the group consisting of G37R, A76I, L129K, N133Q, G141R, P153D, A172E, L180S and K192A; and wherein the variant has DNase activity; and wherein position numbers are based on the numbering of SEQ ID NO: 1. In a particularly preferred embodiment, the variant comprises SEQ ID NO: 1 with the substitution V148I, and at least one substitution, e.g., at least two, at least three, or at least four substitutions, selected from the group consisting of N133Q, P153D, A172E, L180S and K192A, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
V148I+P153D+A172E+K192A, and
N133Q+V148I+L180S;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

### A149F

In one aspect, the variants comprise the substitution A149F of SEQ ID NO: 1. The substitution confers improved detergent stability to the variants.

In a preferred embodiment, the variant comprises SEQ ID NO: 1 with the substitution A149F, and at least one substitution, e.g., at least two, at least three, at least four, or at least five substitutions, selected from the group consisting of G37R, A76I, K86V, K95I, L129K, G141R, S144E, S154E, K155F, L, A172E and D197S; and wherein the variant has DNase activity; and wherein position numbers are based on the numbering of SEQ ID NO: 1. In a particularly preferred embodiment, the variant comprises SEQ ID NO: 1 with the substitution A149F, and at least one substitution, e.g., at least two, at least three, or at least four substitutions, selected from the group consisting of K86V, K95I, S144E, S154E, K155F, L, A172E and D197S, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
K86V+A149F+K155F+A172E,
S144E+A149F+K155L+D197S, and
K95I+S144E+A149F+S154E;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

### P153D

In one aspect, the variants comprise the substitution P153D of SEQ ID NO: 1. The substitution confers improved detergent stability to the variants.

In a preferred embodiment, the variant comprises SEQ ID NO: 1 with the substitution P153D, and at least one substitution, e.g., at least two, at least three, at least four, or at least five substitutions, selected from the group consisting of G37R, A76I, L129K, G141R, V148I, A149E, A172E, K192A and Q208V; and wherein the variant has DNase activity; and wherein position numbers are based on the numbering of SEQ ID NO: 1. In a particularly preferred embodiment, the variant comprises SEQ ID NO: 1 with the substitution P153D, and at least one substitution, e.g., at least two, at least three, or at least four substitutions, selected from the group consisting of V148I, A149E, A172E, K192A and Q208V, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
V148I+P153D+A172E+K192A, and
A149E+P153D+A172E+Q208V;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

### S154E

In one aspect, the variants comprise the substitution S154E of SEQ ID NO: 1. The substitution confers improved detergent stability to the variants.

In a preferred embodiment, the variant comprises SEQ ID NO: 1 with the substitution S154E, and at least one substitution, e.g., at least two, at least three, at least four, or at least five substitutions, selected from the group consisting of G37R, A76I, K86N, K95I, Q102E, L129K, N140H, G141R, S144E, N146A, K147E,N, A149F, Q150D, K160D and D197S; and wherein the variant has DNase activity; and wherein position numbers are based on the numbering of SEQ ID NO: 1. In a particularly preferred embodiment, the variant comprises SEQ ID NO: 1 with the substitution S154E, and at least one substitution, e.g., at least two, at least three, at least four, or at least five substitutions, selected from the group consisting of K86N, K95I, Q102E, N140H, S144E, N146A, K147E,N, A149F, Q150D, K160D and D197S, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
K147N+Q150D+S154E+D197S,
K86N+N146A+Q150D+S154E,
Q102E+N146A+S154E+K160D,
K95I+S144E+A149F+S154E, and
K105N+N140H+K147E+S154E;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

### K155E,F,L,S,T

In one aspect, the variants comprise the substitution K155E,F,L,S,T of SEQ ID NO: 1. The substitution confers improved detergent stability to the variants.

In a preferred embodiment, the variant comprises SEQ ID NO: 1 with the substitution K155E, and at least one substitution, e.g., at least two, at least three, at least four, or at least five substitutions, selected from the group consisting of D32Q, G37R, N68D, A76I, K82T, K86Q, V, K95I, L129K, N140H, G141R, S144E, K147N, A149E, A172D, K192A, D197S, and N217A; and wherein the variant has DNase activity; and wherein position numbers are based on the numbering of SEQ ID NO: 1. In a particularly preferred embodiment, the variant comprises SEQ ID NO: 1 with the substitution K155E, and at least one substitution, e.g., at least two, at least three, at least four, or at least five substitutions, selected from the group consisting of D32Q, N68D, K82T, K86Q, V, K95I, N140H, S144E, K147N, A149E, A172D, K192A, D197S, and N217A, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
K86V+K95I+K155E,
K155E+D197S+N217A,
K82T+N140H+K155E,
K86Q+A149E+K155E+A172D,
N68D+N140H+K147N+K155E,
D32Q+K86V+K155E, and
S144E+A149E+K155E+K192A;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

In a preferred embodiment, the variant comprises SEQ ID NO: 1 with the substitution K155F, and at least one alteration, e.g., at least two, at least three, at least four, at least five, or at least six alterations, selected from the group consisting of, G37R, F43W, A76I, K82S, K86Q,V, K105N,Q, L129K, N140H, G141R, K147E, A149F, A172E, N191*, E211P, N214D and N217A; and wherein the variant has DNase activity; and wherein position numbers are based on the numbering of SEQ ID NO: 1. In a particularly preferred embodiment, the variant comprises SEQ ID NO: 1 with the substitution K155F, and at least one alteration, e.g., at least two, at least three, at least four, or at least five alterations, selected from the group consisting of F43W, K82S, K86Q,V, K105N,Q, N140H, K147E, A149F, A172E, N191*, E211P, N214D and N217A, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
K86V+A149F+K155F+A172E,
F43W+K155F+N217A,
K105Q+K155F+N191*,
K105N+K155F+E211P,
K86Q+N140H+K147E+K155F, and
K82S+K155F+N214D;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

In a preferred embodiment, the variant comprises SEQ ID NO: 1 with the substitution K155L, and at least one substitution, e.g., at least two, at least three, at least four, at least five, or at least six substitutions, selected from the group consisting of G37R, A76I, P84D, K86L, V, K95I, P97E, K105N, L129K, N140H, G141R, S144E, A149E,F, K160D, A172D,H, and D197K,S; and wherein the variant has DNase activity; and wherein position numbers are based on the numbering of SEQ ID NO: 1. In a particularly preferred embodiment, the variant comprises SEQ ID NO: 1 with the substitution K155L, and at least one substitution, e.g., at least two, at least three, at least four, or at least five substitutions, selected from the group consisting of P84D, K86L, V, K95I, P97E, K105N, N140H, S144E, A149E,F, K160D, A172D,H, and D197K,S, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
P84D+K155L+K160D+A172D,
K105N+K155L+D197K,
S144E+A149F+K155L+D197S,
K86V+K95I+K155L,
P97E+N140H+A149E+K155L, and
K86L+K155L+A172H;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

In a preferred embodiment, the variant comprises SEQ ID NO: 1 with the substitution K155S, and at least one substitution, e.g., at least two, at least three, at least four, or at least five substitutions, selected from the group consisting of G37R, A40H, A76I, K86G, T, K105Q, T, L129K, G141R, V187N and E211T; and wherein the variant has DNase activity; and wherein position numbers are based on the numbering of SEQ ID NO: 1. In a particularly preferred embodiment, the variant comprises SEQ ID NO: 1 with the substitution K155S, and at least one substitution, e.g., at least two, at least three, or at least four substitutions, selected from the group consisting of A40H, K86G, T, K105Q, T, V187N and E211T, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
K86G+K105Q+K155S,
K105T+K155S+V187N,
A40H+K155S+V187N, and
K86T+K155S+E211T;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

In a preferred embodiment, the variant comprises SEQ ID NO: 1 with the substitution K155T, and at least one substitution, e.g., at least two, at least three, at least four, or at least five substitutions, selected from the group consisting of G37R, A55I, A76I, K86V, A91R, K105Q, L129K, G141R, A149E and E211T; and wherein the variant has DNase activity; and wherein position numbers are based on the numbering of SEQ ID NO: 1. In a particularly preferred embodiment, the variant comprises SEQ ID NO: 1 with the substitution K155T, and at least one substitution, e.g., at least two, at least three, or at least four substitutions, selected from the group consisting of A55I, K86V, A91R, K105Q, A149E and E211T, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
K105Q+K155T+E211T,
A55I+K86V+K155T, and
A91R+A149E+K155T;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

### Q157D

In one aspect, the variants comprise the substitution Q157D of SEQ ID NO: 1. The substitution confers improved detergent stability to the variants.

In a preferred embodiment, the variant comprises SEQ ID NO: 1 with the substitution Q157D, and at least one substitution, e.g., at least two, at least three, at least four, or at least five substitutions, selected from the group consisting of G37R, A76I, L92E, L129K, G141R, K147N and Q150D; and wherein the variant has DNase activity; and wherein position numbers are based on the numbering of SEQ ID NO: 1. In a particularly preferred embodiment, the variant comprises SEQ ID NO: 1 with the substitution Q157D, and at least one substitution, e.g., at least two, or at least three substitutions, selected from the group consisting of L92E, K147N and Q150D, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises the following set of alterations: L92E+K147N+Q150D+Q157D; and wherein the variant having the above set of alterations may further comprise at least one additional substitution disclosed herein, e.g. at least one substitution selected from the group consisting of G37R, A76I, L129K and G141R; wherein the variant has DNase activity; and wherein position numbers are based on the numbering of SEQ ID NO: 1.

### Q158D

In one aspect, the variants comprise the substitution Q158D of SEQ ID NO: 1. The substitution confers improved detergent stability to the variants.

In a preferred embodiment, the variant comprises SEQ ID NO: 1 with the substitution Q158D, and at least one substitution, e.g., at least two, at least three, at least four, or at least five substitutions, selected from the group consisting of G37R, A76I, P84D, L129K, G141R, N140H and K147E; and wherein the variant has DNase activity; and wherein position numbers are based on the numbering of SEQ ID NO: 1. In a particularly preferred embodiment, the variant comprises SEQ ID NO: 1 with the substitution Q158D, and at least one substitution, e.g., at least two or at least three substitutions, selected from the group consisting of P84D, N140H and K147E; and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises the following set of alterations: P84D+N140H+K147E+Q158D; and wherein the variant having the above set of alterations may further comprise at least one additional substitution disclosed herein, e.g. at least one substitution selected from the group consisting of G37R, A76I, L129K and G141R; wherein the variant has DNase activity; and wherein position numbers are based on the numbering of SEQ ID NO: 1.

### K160D

In one aspect, the variants comprise the substitution K160D of SEQ ID NO: 1. The substitution confers improved detergent stability to the variants.

In a preferred embodiment, the variant comprises SEQ ID NO: 1 with the substitution K160D, and at least one substitution, e.g., at least two, at least three, at least four, or at least five substitutions, selected from the group consisting of G37R, A76I, P84D, Q102E, L129K, G141R, N146A, S154E, K155L and A172D; and wherein the variant has DNase activity; and wherein position numbers are based on the numbering of SEQ ID NO: 1. In a particularly preferred embodiment, the variant comprises SEQ ID NO: 1 with the substitution K160D, and at least one substitution, e.g., at least two, or at least three substitutions, selected from the group consisting of P84D, Q102E, N146A, S154E, K155L and A172D, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
P84D+K155L+K160D+A172D, and
Q102E+N146A+S154E+K160D;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

### A172D,E

In one aspect, the variants comprise the substitution A172D or A172E of SEQ ID NO: 1. The substitution confers improved detergent stability to the variants.

In a preferred embodiment, the variant comprises SEQ ID NO: 1 with the substitution A172D, and at least one substitution, e.g., at least two, at least three, at least four, at least five, or at least six substitutions, selected from the group consisting of G37R, A76I, P84D, K86Q, K95I, L129K, G141R, K147N, A149E, K155E,L, K160D, V187N and K192A; and wherein the variant has DNase activity; and wherein position numbers are based on the numbering of SEQ ID NO: 1. In a particularly preferred embodiment, the variant comprises SEQ ID NO: 1 with the substitution A172D, and at least one substitution, e.g., at least two, or at least three substitutions, selected from the group consisting of P84D, K86Q, K95I, K147N, A149E, K155E,L, K160D, V187N and K192A, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
P84D+K155L+K160D+A172D,
K86Q+A149E+K155E+A172D,
K95I+A172D+V187N+K192A, and
K147N+A172D+V187N+K192A;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

In a preferred embodiment, the variant comprises SEQ ID NO: 1 with the substitution A172E, and at least one substitution, e.g., at least two, at least three, at least four, at least five, or at least six substitutions, selected from the group consisting of G37R, A76I, K86V, A101E, K105G, F112Y, L129K, G141R, K147E, V148I, A149E,F, P153D, K155F, V187N, K192A, Q208V, N214D and Y218E; and wherein the variant has DNase activity; and wherein position numbers are based on the numbering of SEQ ID NO: 1. In a particularly preferred embodiment, the variant comprises SEQ ID NO: 1 with the substitution A172E, and at least one substitution, e.g., at least two, at least three or at least four substitutions, selected from the group consisting of K86V, A101E, K105G, F112Y, K147E, V148I, A149E,F, P153D, K155F, V187N, K192A, Q208V, N214D and Y218E, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
K86V+A149F+K155F+A172E,
V148I+P153D+A172E+K192A,
A149E+P153D+A172E+Q208V,
K105G+F112Y+A172E,
A101E+A172E+N214D+Y218E, and
K147E+A172E+V187N+K192A;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

### V187N,Y

In one aspect, the variants comprise the substitution V187N or V187Y of SEQ ID NO: 1. The substitution confers improved detergent stability to the variants.

In a preferred embodiment, the variant comprises SEQ ID NO: 1 with the substitution V187N, and at least one substitution, e.g., at least two, at least three, at least four, at least five, or at least six substitutions, selected from the group consisting of G37R, A40H, S69V, A76I, K86T, L92E, K95I, K105N,Q,T, L129K, G141R, K147E,N, K155S, A172D,E,R, K192A, D197S and Y209H; and wherein the variant has DNase activity; and wherein position numbers are based on the numbering of SEQ ID NO: 1. In a particularly preferred embodiment, the variant comprises SEQ ID NO: 1 with the substitution V187N, and at least one substitution, e.g., at least two, at least three or at least four substitutions, selected from the group consisting of A40H, S69V, K86T, L92E, K95I, K105N,Q,T, K147E,N, K155S, A172D,E,R, K192A, D197S and Y209H, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
K95I+A172D+V187N+K192A,
K105Q+V187N+K192A+D197S,
K147N+A172D+V187N+K192A,
K105N+A172R+V187N,
K105T+K155S+V187N,
S69V+K86T+L92E+V187N,
K147E+A172E+V187N+K192A,
K105Q+V187N+Y209H, and
A40H+K155S+V187N;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

In a preferred embodiment, the variant comprises SEQ ID NO: 1 with the substitution V187Y, and at least one substitution, e.g., at least two, at least three, at least four, at least five, or at least six substitutions, selected from the group consisting of G37R, A76I, K86G,L, A101E, L129K, G141R and T170Q; and wherein the variant has DNase activity; and wherein position numbers are based on the numbering of SEQ ID NO: 1. In a particularly preferred embodiment, the variant comprises SEQ ID NO: 1 with the substitution V187Y, and at least one substitution, e.g., at least two or at least three substitutions, selected from the group consisting of K86G,L, A101E and T170Q, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
K86G+A101E+V187Y, and
K86L+T170Q+V187Y;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

### K192A

In one aspect, the variants comprise the substitution K192A of SEQ ID NO: 1. The substitution confers improved detergent stability to the variants.

In a preferred embodiment, the variant comprises SEQ ID NO: 1 with the substitution K192A, and at least one substitution, e.g., at least two, at least three, at least four, at least five, or at least six substitutions, selected from the group consisting of G37R, S69V, A76I, K86Q, L92E, K95I, K105Q, L129K, G141R, S144E, K147E,N, V148I, A149E, P153D, K155E, A172D,E, V187N and D197S; and wherein the variant has DNase activity; and wherein position numbers are based on the numbering of SEQ ID NO: 1. In a particularly preferred embodiment, the variant comprises SEQ ID NO: 1 with the substitution K192A, and at least one substitution, e.g., at least two, at least three or at least four substitutions, selected from the group consisting of S69V, K86Q, L92E, K95I, K105Q, S144E, K147E,N, V148I, A149E, P153D, K155E, A172D,E, V187N and D197S, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
S69V+K86Q+L92E+K192A,
V148I+P153D+A172E+K192A,
K95I+A172D+V187N+K192A,
K105Q+V187N+K192A+D197S,
K147N+A172D+V187N+K192A,
K147E+A172E+V187N+K192A, and
S144E+A149E+K155E+K192A;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

### D197S

In one aspect, the variants comprise the substitution D197S of SEQ ID NO: 1. The substitution confers improved detergent stability to the variants.

In a preferred embodiment, the variant comprises SEQ ID NO: 1 with the substitution D197S, and at least one substitution, e.g., at least two, at least three, at least four, at least five, or at least six substitutions, selected from the group consisting of G37R, A76I, K105Q, L129K, N133Q, G141R, S144E, N146A, K147N, A149F, Q150D, P153D, S154E, K155E,L, V187N, K192A and N217A; and wherein the variant has DNase activity; and wherein position numbers are based on the numbering of SEQ ID NO: 1. In a particularly preferred embodiment, the variant comprises SEQ ID NO: 1 with the substitution D197S, and at least one substitution, e.g., at least two, at least three or at least four substitutions, selected from the group consisting of K105Q, N133Q, S144E, N146A, K147N, A149F, Q150D, P153D, S154E, K155E,L, V187N, K192A and N217A, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
K155E+D197S+N217A,
S144E+A149F+K155L+D197S,
K147N+Q150D+S154E+D197S,
K105Q+V187N+K192A+D197S, and
N133Q+N146A+P153D+D197S;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

### Q208V

In one aspect, the variants comprise the substitution Q208V of SEQ ID NO: 1. The substitution confers improved detergent stability to the variants.

In a preferred embodiment, the variant comprises SEQ ID NO: 1 with the substitution Q208V, and at least one substitution, e.g., at least two, at least three, at least four, at least five, or at least six substitutions, selected from the group consisting of G37R, A76I, L129K, G141R, A149E, P153D and A172E; and wherein the variant has DNase activity; and wherein position numbers are based on the numbering of SEQ ID NO: 1. In a particularly preferred embodiment, the variant comprises SEQ ID NO: 1 with the substitution Q208V, and at least one substitution, e.g., at least two or at least three substitutions, selected from the group consisting of A149E, P153D and A172E, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises the following set of alterations: A149E+P153D+A172E+Q208V; and wherein the variant having the above set of alterations may further comprise at least one additional substitution disclosed herein, e.g. at least one substitution selected from the group consisting of G37R, A76I, L129K and G141R; wherein the variant has DNase activity; and wherein position numbers are based on the numbering of SEQ ID NO: 1.

### N214D

In one aspect, the variants comprise the substitution N214D of SEQ ID NO: 1. The substitution confers improved detergent stability to the variants.

In a preferred embodiment, the variant comprises SEQ ID NO: 1 with the substitution N214D, and at least one substitution, e.g., at least two, at least three, at least four, at least five, or at least six substitutions, selected from the group consisting of G37R, A76I, K82S, A101E, Q102E, K105D,G,N, F112W, L129K, G141R, A149E, K155F, A172E and Y218D, E; and wherein the variant has DNase activity; and wherein position numbers are based on the numbering of SEQ ID NO: 1. In a particularly preferred embodiment, the variant comprises SEQ ID NO: 1 with the substitution N214D, and at least one substitution, e.g., at least two, at least three or at least four substitutions, selected from the group consisting of K82S, A101E, Q102E, K105D,G,N, F112W, A149E, K155F, A172E and Y218D,E, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
A149E+N214D+Y218E,
A101E+N214D+Y218E,
A149E+N214D+Y218D,
K105G+N214D+Y218E,
A101E+N214D+Y218D,
Q102E+N214D+Y218E,
F112W+N214D+Y218E,
Q102E+N214D+Y218D,
F112W+N214D+Y218D,
A101E+A172E+N214D+Y218E,
A101E+K105N+N214D+Y218E,
K82S+K155F+N214D, and
K105D+N214D+Y218E;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

### N217A

In one aspect, the variants comprise the substitution N217A of SEQ ID NO: 1. The substitution confers improved detergent stability to the variants.

In a preferred embodiment, the variant comprises SEQ ID NO: 1 with the substitution N217A, and at least one substitution, e.g., at least two, at least three, at least four, at least five, or at least six substitutions, selected from the group consisting of G37R, F43W, A76I, L129K, G141R, K155E,F and D197S; and wherein the variant has DNase activity; and wherein position numbers are based on the numbering of SEQ ID NO: 1. In a particularly preferred embodiment, the variant comprises SEQ ID NO: 1 with the substitution N217A, and at least one substitution, e.g., at least two, at least three or at least four substitutions, selected from the group consisting of F43W, K155E,F and D197S, and optionally at least one substitution, e.g., at least two, at least three, or at least four substitutions, selected from the group consisting of G37R, A76I, L129K and G141R; and wherein the variant has DNase activity; and wherein position numbers are based on the numbering of SEQ ID NO: 1. These substitutions confer improved detergent stability and/or activity to the variants.

In a preferred embodiment, the variant has a sequence identity of at least 80%, e.g., at least 85%, at least 90%, at least 95%, at least 96%, at least 97% or at least 98%, but less than 100%, to the parent polypeptide of SEQ ID NO: 1, SEQ ID NO: 2 or SEQ ID NO: 3; wherein the variant comprises one of the following sets of alterations:
K155E+D197S+N217A, and
F43W+K155F+N217A;
wherein the variant having one of the above sets of alterations may further comprise at least one additional substitution disclosed herein, e.g. at least one substitution selected from the group consisting of G37R, A76I, L129K and G141R;
wherein the variant has DNase activity; and wherein position numbers are based on the numbering of SEQ ID NO: 1.

In one preferred embodiment, the DNase variant of the invention comprises at least one substitution selected from the group consisting of S69V, A101E, Q102E, P153V, Q157E, T159Q, A172E, N214D and Y218D, for example one, two, three or four of said substitutions. More preferably, the DNase variant of this embodiment comprises at least one substitution selected from the group consisting of P153V, Q157E, T159Q and A172E, for example one or two of said substitutions. Examples of such variants include the following:
S69V,Q102E,T159Q
A101E,A172E,N214D,Y218E
A101E,K105N,N214D,Y218E
S69V,Q102E,Q157E,T159Q
S69V,Q102E,P153V,T159Q

In preferred embodiments, the DNase variant of the present invention comprises at least one of the following sets of alterations as shown in below table, and may optionally further comprise at least one substitution selected from the group consisting of G37R, A76I, L129K and G141R; and wherein the variant has DNase activity, and wherein the positions correspond to the positions of SEQ ID NO: 1.

| | |
|---|---|
| 1 | N133Q, V148I, L180S |
| 2 | D46G, N133Q, K185* |
| 3 | K86L, K155L, A172H |
| 4 | K86T, K155S, E211T |
| 5 | K86T, K155E, A172R |
| 6 | K86L, T170Q, V187Y |
| 7 | K36H, K86T, K155E |
| 8 | A91R, A149E, K155T |
| 9 | K86Y, Q102E, A149E |
| 10 | A40H, K155S, V187N |
| 11 | K105Q, V187N, Y209H |
| 12 | A55I, K86V, K155T |
| 13 | S144E, A149E, K155E, K192A |
| 14 | K105D, N214D, Y218E |
| 15 | K147E, A172E, V187N, K192A |
| 16 | S69V, Q102E, T159Q |
| 17 | K36C, V138C |
| 18 | L92E, K147N, Q150D, Q157D |
| 19 | S69V, K86T, L92E, V187N |
| 20 | K82S, K155F, N214D |
| 21 | N133Q, N146A, P153D, D197S |
| 22 | S69V, G79C, P107C |
| 23 | K105G, F112Y, A172E |
| 24 | K86Q, N140H, K147E, K155F |
| 25 | F112W, N214D, Y218D |
| 26 | K105N, N140H, K147E, S154E |
| 27 | Q102E, N214D, Y218D |
| 28 | D32Q, K86V, K155E |
| 29 | F112W, N214D, Y218E |
| 30 | K86L, K95I, A149E |
| 31 | K86L, A101E, E211Y |
| 32 | A101E, N214D, Y218D |
| 33 | A101E, N214D, Y218E |
| 34 | A149E, N214D, Y218D |
| 35 | A149E, N214D, Y218E |
| 36 | A149E, P153D, A172E, Q208V |
| 37 | D46G, A101E, G141Q |
| 38 | F43W, K155F, N217A |
| 39 | K105G, N214D, Y218E |
| 40 | K105N, A149D, N213S |
| 41 | K105N, A172R, V187N |
| 42 | K105N, K155F, E211P |
| 43 | K105N, K155L, D197K |
| 44 | K105Q, K155F, N191* |
| 45 | K105Q, K155T, E211T |
| 46 | K105Q, V187N, K192A, D197S |
| 47 | K105T, K155S, V187N |
| 48 | K147N, A172D, V187N, K192A |
| 49 | K147N, Q150D, S154E, D197S |
| 50 | K155E, D197S, N217A |
| 51 | K36C, K105G, V138C |
| 52 | K82T, N140H, K155E |
| 53 | K86G, A101E, V187Y |
| 54 | K86G, K105Q, K155S |
| 55 | K86N, N146A, Q150D, S154E |
| 56 | K86Q, A101E, K105Q, N146A |
| 57 | K86Q, A149E, K155E, A172D |
| 58 | K86T, A101E, G199Q |
| 59 | K86V, A149F, K155F, A172E |
| 60 | K86V, K95I, K155E |
| 61 | K86V, K95I, K155L |
| 62 | K95I, A172D, V187N, K192A |
| 63 | K95I, S144E, A149F, S154E |
| 64 | N68D, N140H, K147N, K155E |
| 65 | P84D, K155L, K160D, A172D |
| 66 | P84D, N140H, K147E, Q158D |
| 67 | P84T, A101E, F112Y |
| 68 | P97E, N140H, A149E, K155L |
| 69 | Q102E, N146A, S154E, K160D |
| 70 | Q102E, N214D, Y218E |
| 71 | S144E, A149F, K155L, D197S |
| 72 | S26*, K105N, G141Q |
| 73 | S69V, K86Q, L92E, K192A |
| 74 | V148I, P153D, A172E, K192A |
| 75 | A101E, A172E, N214D, Y218E |
| 76 | A101E, K105N, N214D, Y218E |
| 77 | S69V, Q102E, P153V, T159Q |
| 78 | S69V, Q102E, Q157E, T159Q |

The DNase variants of the present invention may have improved DNase activity, such as improved specific activity, compared to a reference DNase, e.g. the DNase of SEQ ID NO: 1.

Preferably, the DNase variants of the present invention have improved stability in detergent, in particular improved storage stability in a liquid detergent, compared to a reference DNase e.g. SEQ ID NO: 1, wherein stability may be tested as described in Example 2.

In a preferred embodiment, a DNase variant of the present invention having two or more mutations has improved stability in detergent (e.g. as described in Example 2) compared to at least one single mutation variant in the corresponding positions. For example, the stability of the DNase variant with substitutions of K86Q, A101E, K105Q and N146A is higher than the stability of a single mutation variant selected from the group consisting of K86Q, A101E, K105Q and N146A.

In a preferred embodiment, the DNase variant of the present invention has improved stability and improved activity compared to a reference DNase e.g. SEQ ID NO: 1.

In one embodiment, the DNase variants of the present invention have improved deep cleaning performance compared to a reference DNase e.g. SEQ ID NO: 1.

When items like T-shirts or sportswear are used, they are exposed to bacteria from the body of the user and from the rest of the environment in which they are used. This may cause malodor on the item even after the item is washed. The present invention therefore also relates to methods for removal or reduction of malodor on textile. The malodor may be caused by bacteria producing compounds with an unpleasant smell. One example of such unpleasant smelling compounds is E-2-nonenal. The malodor can be present on newly washed textile which is still wet, or the malodor can be present on newly washed textile which has subsequently been dried. The malodor may also be present on textile which has been stored for some time after wash. The present invention thus also relates to use of a DNase variant of the invention for reduction or removal of malodor such as E-2-nonenal from wet or dry textiles.

In one embodiment, the DNase variants of the present invention have improved malodor removal properties compared to a reference DNase e.g. SEQ ID NO: 1, wherein the malodor is measured as described in Assay II.

Further, the invention relates to the use of a DNase variant of the present invention for preventing, reducing or removing the adherence of soil to an item. In one embodiment, the item is textile. When the soil does not adhere to the item, the item appears cleaner. Thus, the present invention further concerns the use of a DNase variant for maintaining or improving the whiteness of the item.

The present invention further concerns detergent compositions comprising a DNase variant according to the invention and at least one detergent adjunct ingredient, e.g. a surfactant.

The detergent composition comprising a DNase variant according to the invention may be used for preventing, reducing or removing biofilm from an item, for preventing, reducing or removing the stickiness of an item, for pretreating stains on the item, for preventing, reducing redeposition of soil during a wash cycle, for preventing, reducing or removing adherence of soil to an item, for maintaining or improving the whiteness of an item, or for preventing, reducing or removing malodor (such as E-2-nonenal) from an item (as described in Assay II).

In one embodiment, the detergent composition is a liquid or powder laundry detergent, suitable for e.g. washing at high temperature and/or pH, such as at or above 40° C. and/or at or above pH 8. In one embodiment the detergent composition is a liquid or powder laundry detergent, suitable for e.g. washing at low temperature and/or pH, such as at or below 20° C. and/or at pH 6-7.

The detergent composition may also be formulated as a unit dose detergent and/or compact detergent optionally with minimum or no water. The detergent may also be a dishwashing detergent. The laundry and dishwashing detergents are preferably phosphate-free. The detergent composition may further comprise at least one additional enzyme, such as carbohydrate-active enzymes like carbohydrase, pectinase, mannanase, amylase, cellulase, arabinase, galactanase, lichenase, xylanase, protease such as metalloproteases, lipase, cutinase, oxidase, e.g., a laccase, and/or peroxidase.

In some further embodiments, the present invention relates to DNase variants having at least 80% to SEQ ID NO: 1 identity hereto wherein the variant having at least one improved property compared to SEQ ID NO: 1 when tested in a relevant assay. In one embodiment, the property is stability, such as storage stability in detergent. In another embodiment the property is wash performance, such as deep cleaning performance.

In one embodiment, the variants according to the invention have improved stability relative to the parent, e.g. SEQ ID NO: 1, e.g. measured as residual activity.

The variants according to the invention may further comprise one or more additional alterations at one or more other positions. The amino acid changes may be of a minor nature, that is conservative amino acid substitutions or insertions that do not significantly affect the folding and/or activity of the protein; small deletions, typically of 1-5 amino acids; small amino- or carboxyl-terminal extensions, such as an amino-terminal methionine residue; a small linker peptide of up to 20-25 residues, located at the amino- or carboxyl terminal; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Asn/Gln, Thr/Ser, Ala/Gly, Ala/Thr, Ser/Asn, Ala/Val, Ser/Gly, Tyr/Phe, Ala/Pro, Lys/Arg, Asp/Asn, Glu/Gln, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for DNase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Although the changes described above preferably are of a minor nature, such changes may also be of a substantive nature such as fusion of larger polypeptides of up to 300 amino acids or more both as amino- or carboxyl-terminal extensions.

### Parent

The parent DNase may comprise or consist of the amino acid sequence of SEQ ID NO: 1 or an allelic variant thereof; or a fragment thereof having DNase activity. In one aspect, the parent DNase comprises or consists of the amino acid sequence of SEQ ID NO: 1.

In a preferred aspect of the present invention, the parent DNase comprise a polypeptide having at least 60%, such as at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 1, SEQ ID NO: 2 or SEQ ID NO: 3.

The parent DNase may be obtained from organisms of any genus. For purposes of the present invention, the term “obtained from” as used herein in connection with a given source shall mean that the parent encoded by a polynucleotide is produced by the source or by a strain in which the polynucleotide from the source has been inserted. In one aspect, the parent is secreted extracellularly.

The parent may be a fungal DNase. For example, the parent may be an Aspergillus DNase. In one aspect, the parent is an Aspergillus oryzae DNase, e.g., a DNase with SEQ ID NO: 1.

Strains of these species are readily accessible to the public in a number of culture collections, such as the American Type Culture Collection (ATCC), Deutsche Sammlung von Mikroorganismen und Zellkulturen GmbH (DSMZ), Centraalbureau Voor Schimmelcultures (CBS), and Agricultural Research Service Patent Culture Collection, Northern Regional Research Center (NRRL).

The parent may be identified and obtained from other sources including microorganisms isolated from nature (e.g., soil, composts, water, etc.) or DNA samples obtained directly from natural materials (e.g., soil, composts, water, etc.) using nucleic acid probes designed on the basis of a polynucleotide encoding the DNase of SEQ ID NO: 1 as is known in the art. Techniques for isolating microorganisms and DNA directly from natural habitats are well known in the art. A polynucleotide encoding a parent may then be obtained by similarly screening a genomic DNA or cDNA library of another microorganism or mixed DNA sample. Once a polynucleotide encoding a parent has been detected with the probe(s), the polynucleotide can be isolated or cloned by utilizing techniques that are known to those of ordinary skill in the art (see, e.g., Sambrook et al., 1989, supra).

### Preparation of Variants

The present invention also relates to a method for obtaining a DNase variant having at least one improved property compared to SEQ ID NO: 1, comprising:
a) introducing into a DNase at least two alterations at positions selected from the group consisting of positions 26, 32, 36, 37, 43, 46, 55, 68, 69, 76, 79, 82, 84, 86, 91, 92, 95, 97, 101, 102, 105, 107, 112, 129, 133, 138, 140, 141, 144, 146, 147, 148, 149, 150, 153, 154, 155, 157, 158, 159, 160, 170, 172, 185, 187, 191, 192, 197, 199, 208, 211, 213, 214, 217 and 218 of SEQ ID NO: 1, wherein the variant has a sequence identity of at least 80%, but less than 100%, to the polypeptide of SEQ ID NO: 1, SEQ ID NO: 2 or SEQ ID NO: 3; and
b) recovering the variant.

The present invention also relates to a method for obtaining a DNase variant having at least one improved property compared to SEQ ID NO: 1, comprising
a) introducing into a DNase at least two alterations selected from the group consisting of S26*, D32Q, K36C, H, G37R, F43W, D46G, A55I, N68D, S69V, A76I, K82S, T, P84D,T, K86G,L,N,Q,T,V,Y, A91R, L92E, K95I, P97E,N, A101E, Q102E, K105N,G,Q,T,D, F112Y,W, L129K, N133Q, V138C, N140H, G141Q,R, S144E, N146A, K147N,E, V148I, A149D,E,F, Q150D, P153D,V, S154E, K155E,F,L,S,T, Q157D,E, Q158D, T159Q, K160D, T170Q, A172D,E,H,R, K185*, V187N, Y, N191*, K192A, D197K,S, G199Q, Q208V, E211Y,T,P, N213S, N214D, N217A and Y218D, E, wherein the variant has a sequence identity of at least 85%, at least 90%, at least 95%, at least 96%, at least 97%, at least 98% or at least 99%, but less than 100%, to the polypeptide of SEQ ID NO: 1, SEQ ID NO: 2 or SEQ ID NO: 3, wherein the positions correspond to the positions of SEQ ID NO: 1; and
b) recovering the variant.

The present invention also relates to a method for obtaining a DNase variant having at least one improved property compared to SEQ ID NO: 1, comprising
a) introducing into a DNase at least two alterations selected from the group consisting of S26*, G37R, S69V, A76I, K86G,L,N,Q,T,V,Y, K95I, A101E, Q102E, K105N,G,Q,T,D, F112Y,W, L129K, N140H, G141Q,R, S144E, N146A, K147N,E, V148I, A149D,E,F, Q150D, P153D,V, S154E, K155E,F,L,S,T, Q157E, Q158D, T159Q, K160D, A172D,E,H,R, V187N,Y, K192A, D197K,S, G199Q, Q208V, E211Y,T,P, N214D, N217A and Y218D, E, wherein the variant has a sequence identity of at least 85%, at least 90%, at least 95%, at least 96%, at least 97%, at least 98% or at least 99%, but less than 100%, to the polypeptide of SEQ ID NO: 1, SEQ ID NO: 2 or SEQ ID NO: 3, wherein the positions correspond to the positions of SEQ ID NO: 1; and
b) recovering the variant.

The present invention also relates to a method for obtaining a DNase variant having at least one improved property compared to SEQ ID NO: 1, comprising
a) introducing into a DNase at least two alterations selected from the group consisting of S26*, G37R, S69V, A76I, K86G,L,N,Q,T,V,Y, K95I, A101E, Q102E, K105N,G,Q,T,D, L129K, G141Q, R, V148I, P153D, V, K155E,F,L,S,T, Q157E, T159Q, A172E, V187N, Y, D197K,S, N214D and Y218D, E, wherein the variant has a sequence identity of at least 85%, at least 90%, at least 95%, at least 96%, at least 97%, at least 98% or at least 99%, but less than 100%, to the polypeptide of SEQ ID NO: 1, SEQ ID NO: 2 or SEQ ID NO: 3, wherein the positions correspond to the positions of SEQ ID NO: 1; and
b) recovering the variant.

The present invention also relates to a method for obtaining a DNase variant having at least one improved property compared to SEQ ID NO: 1, comprising
a) introducing into a DNase at least one of the following sets of alterations:

| | |
|---|---|
| 1 | N133Q, V148I, L180S |
| 2 | D46G, N133Q, K185* |
| 3 | K86L, K155L, A172H |
| 4 | K86T, K155S, E211T |
| 5 | K86T, K155E, A172R |
| 6 | K86L, T170Q, V187Y |
| 7 | K36H, K86T, K155E |
| 8 | A91R, A149E, K155T |
| 9 | K86Y, Q102E, A149E |
| 10 | A40H, K155S, V187N |
| 11 | K105Q, V187N, Y209H |
| 12 | A55I, K86V, K155T |
| 13 | S144E, A149E, K155E, K192A |
| 14 | K105D, N214D, Y218E |
| 15 | K147E, A172E, V187N, K192A |
| 16 | S69V, Q102E, T159Q |
| 17 | K36C, V138C |
| 18 | L92E, K147N, Q150D, Q157D |
| 19 | S69V, K86T, L92E, V187N |
| 20 | K82S, K155F, N214D |
| 21 | N133Q, N146A, P153D, D197S |
| 22 | S69V, G79C, P107C |
| 23 | K105G, F112Y, A172E |
| 24 | K86Q, N140H, K147E, K155F |
| 25 | F112W, N214D, Y218D |
| 26 | K105N, N140H, K147E, S154E |
| 27 | Q102E, N214D, Y218D |
| 28 | D32Q, K86V, K155E |
| 29 | F112W, N214D, Y218E |
| 30 | K86L, K95I, A149E |
| 31 | K86L, A101E, E211Y |
| 32 | A101E, N214D, Y218D |
| 33 | A101E, N214D, Y218E |
| 34 | A149E, N214D, Y218D |
| 35 | A149E, N214D, Y218E |
| 36 | A149E, P153D, A172E, Q208V |
| 37 | D46G, A101E, G141Q |
| 38 | F43W, K155F, N217A |
| 39 | K105G, N214D, Y218E |
| 40 | K105N, A149D, N213S |
| 41 | K105N, A172R, V187N |
| 42 | K105N, K155F, E211P |
| 43 | K105N, K155L, D197K |
| 44 | K105Q, K155F, N191* |
| 45 | K105Q, K155T, E211T |
| 46 | K105Q, V187N, K192A, D197S |
| 47 | K105T, K155S, V187N |
| 48 | K147N, A172D, V187N, K192A |
| 49 | K147N, Q150D, S154E, D197S |
| 50 | K155E, D197S, N217A |
| 51 | K36C, K105G, V138C |
| 52 | K82T, N140H, K155E |
| 53 | K86G, A101E, V187Y |
| 54 | K86G, K105Q, K155S |
| 55 | K86N, N146A, Q150D, S154E |
| 56 | K86Q, A101E, K105Q, N146A |
| 57 | K86Q, A149E, K155E, A172D |
| 58 | K86T, A101E, G199Q |
| 59 | K86V, A149F, K155F, A172E |
| 60 | K86V, K95I, K155E |
| 61 | K86V, K95I, K155L |
| 62 | K95I, A172D, V187N, K192A |
| 63 | K95I, S144E, A149F, S154E |
| 64 | N68D, N140H, K147N, K155E |
| 65 | P84D, K155L, K160D, A172D |
| 66 | P84D, N140H, K147E, Q158D |
| 67 | P84T, A101E, F112Y |
| 68 | P97E, N140H, A149E, K155L |
| 69 | Q102E, N146A, S154E, K160D |
| 70 | Q102E, N214D, Y218E |
| 71 | S144E, A149F, K155L, D197S |
| 72 | S26*, K105N, G141Q |
| 73 | S69V, K86Q, L92E, K192A |
| 74 | V148I, P153D, A172E, K192A |
| 75 | A101E, A172E, N214D, Y218E |
| 76 | A101E, K105N, N214D, Y218E |
| 77 | S69V, Q102E, P153V, T159Q |
| 78 | S69V, Q102E, Q157E, T159Q |

b) and recovering the variant.

The variants of the present invention may also be prepared by procedures such as those mentioned below.

Site-directed mutagenesis is a technique in which one or more mutations are introduced at one or more defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76: 4949-4955; and Barton et al., 1990, Nucleic Acids Res. 18: 7349-4966.

Site-directed mutagenesis can also be accomplished in vivo by methods known in the art. See, e.g., U.S. Patent Application Publication No. 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16.

Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

### Polynucleotides

The present invention also relates to isolated polynucleotides encoding a DNase variant of the present invention.

The techniques used to isolate or clone a polynucleotide are known in the art and include isolation from genomic DNA or cDNA, or a combination thereof. The cloning of the polynucleotides from genomic DNA can be effected, e.g., by using the well-known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned in a strain of Bacillus subtilis or E. coli, or a related organism and thus, for example, may be an allelic or species variant of the polypeptide encoding region of the polynucleotide.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed on the basis of the polynucleotide presented as the mature polypeptide coding sequence of SEQ ID NO: 1, e.g., a subsequence thereof, and/or by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, Ford et al., (1991), ‘Protein Expression and Purification’, 2: 95-107.

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xyIA and xyIB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2 tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO 1), Saccharomyces cerevisiae galactokinase (GAL1), Saccharomyces cerevisiae alcohol dehydrogenase/glyceraldehyde-3 phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3 phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′ terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3 phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene. Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′ terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO 1), Saccharomyces cerevisiae 3 phosphoglycerate kinase, Saccharomyces cerevisiae alpha-factor, and Saccharomyces cerevisiae alcohol dehydrogenase/glyceraldehyde-3 phosphate dehydrogenase (ADH2/GAP).

The control sequence may also be a polyadenylation sequence; a sequence operably linked to the 3′ terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N terminus of a polypeptide and directs the polypeptide into the cell's secretory pathway. The 5′ end of the coding sequence of the polynucleotide may inherently contain a signal peptide coding sequence naturally linked in translation reading frame with the segment of the coding sequence that encodes the polypeptide. Alternatively, the 5′ end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N terminus of a polypeptide and the signal peptide sequence is positioned next to the N terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression. In a further embodiment, polynucleotide sequence codons have been modified by nucleotide substitutions to correspond to the codon usage of the host organism intended for production of the polypeptide of the present invention. The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosylaminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′ phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome. For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMß1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a polypeptide of the present invention, comprising (a) cultivating a cell, which in its wild-type form produces the polypeptide, under conditions conducive for production of the polypeptide; and optionally (b) recovering the polypeptide. In one aspect, the cell is an Aspergillus cell. In another aspect, the cell is an Aspergillus oryzae cell.

The present invention also relates to methods of producing a polypeptide of the present invention, comprising (a) cultivating a recombinant host cell of the present invention under conditions conducive for production of the polypeptide; and optionally (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

In one embodiment, the invention further comprises producing the polypeptide by cultivating the recombinant host cell further comprising a polynucleotide encoding a second polypeptide of interest; preferably an enzyme of interest; more preferably a secreted enzyme of interest; even more preferably a hydrolase, isomerase, ligase, lyase, oxidoreductase, or a transferase; and most preferably the secreted enzyme is an alpha-galactosidase, alpha-glucosidase, aminopeptidase, amylase, asparaginase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, green fluorescent protein, glucano-transferase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or a xylanase.

In one embodiment, the second polypeptide of interest is heterologous or homologous to the host cell.

In one embodiment, the recombinant host cell is a fungal host cell; preferably a filamentous fungal host cell; more preferably an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell; most preferably an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

In one embodiment, the recombinant host cell is a bacterial host cell; preferably a prokaryotic host cell; more preferably a Gram-positive host cell; even more preferably a Bacillus, Clostridium, Enterococcus, Geobacillus, Lactobacillus, Lactococcus, Oceanobacillus, Staphylococcus, Streptococcus, or Streptomyces host cell; and most preferably a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis host cell.

In one embodiment, a method of producing the second polypeptide of interest comprises cultivating the host cell under conditions conducive for production of the second polypeptide of interest.

In one embodiment, the method further comprises recovering the second polypeptide of interest.

### Compositions

The present invention further concerns a composition comprising at least one DNase variant according to the invention and preferably a detergent adjunct ingredient e.g. a surfactant. In one embodiment, the composition is a detergent composition which may be used for preventing, reducing or removing biofilm from an item, for preventing, reducing or removing the stickiness of an item, for pretreating stains on the item, for preventing, reducing or removing redeposition of soil during a wash cycle, for reducing or removing adherence of soil to an item, for maintaining or improving the whiteness of an item and for preventing, reducing or removing malodor from an item, such as E-2-nonenal as described in Assay II. The DNase variants of the present invention are useful in powder and liquid detergent.

One embodiment of the present invention relates to a detergent composition comprising a DNase variant which compared to SEQ ID NO: 1 comprises at least two alterations at positions selected from the group consisting of positions 26, 32, 36, 37, 43, 46, 55, 68, 69, 76, 79, 82, 84, 86, 91, 92, 95, 97, 101, 102, 105, 107, 112, 129, 133, 138, 140, 141, 144, 146, 147, 148, 149, 150, 153, 154, 155, 157, 158, 159, 160, 170, 172, 185, 187, 191, 192, 197, 199, 208, 211, 213, 214, 217 and 218, wherein the variant has a sequence identity of at least 80%, but less than 100%, to the polypeptide of SEQ ID NO: 1, SEQ ID NO: 2 or SEQ ID NO: 3, and has DNase activity, and a detergent adjunct ingredient.

In one embodiment of the present invention, the detergent composition comprises a DNase variant which compared to SEQ ID NO: 1 comprises at least two alterations selected from the group consisting of S26*, D32Q, K36C,H, G37R, F43W, D46G, A55I, N68D, S69V, A76I, K82S,T, P84D,T, K86G,L,N,Q,T,V,Y, A91R, L92E, K95I, P97E,N, A101E, Q102E, K105N,G,Q,T,D, F112Y,W, L129K, N133Q, V138C, N140H, G141Q,R, S144E, N146A, K147N,E, V148I, A149D,E,F, Q150D, P153D,V, S154E, K155E,F,L,S,T, Q157D,E, Q158D, T159Q, K160D, T170Q, A172D,E,H,R, K185*, V187N, Y, N191*, K192A, D197K,S, G199Q, Q208V, E211Y,T,P, N213S, N214D, N217A and Y218D, E, wherein the variant has a sequence identity of at least 80%, but less than 100%, to the polypeptide of SEQ ID NO: 1, SEQ ID NO: 2 or SEQ ID NO: 3, and has DNase activity, and a detergent adjunct.

In one embodiment of the present invention the detergent composition comprises a DNase variant which compared to SEQ ID NO: 1 comprises at least two alterations selected from the group consisting of S26*, G37R, S69V, A76I, K86G,L,N,Q,T,V,Y, K95I, A101E, Q102E, K105N,G,Q,T,D, F112Y,W, L129K, N140H, G141Q,R, S144E, N146A, K147N,E, V148I, A149D,E,F, Q150D, P153D,V, S154E, K155E,F,L,S,T, Q157E, Q158D, T159Q, K160D, A172D,E,H,R, V187N,Y, K192A, D197K,S, G199Q, Q208V, E211Y,T,P, N214D, N217A and Y218D, E, wherein the variant has a sequence identity of at least 80%, but less than 100%, to the polypeptide of SEQ ID NO: 1, SEQ ID NO: 2 or SEQ ID NO: 3, and has DNase activity, and a detergent adjunct ingredient.

In one embodiment of the present invention the detergent composition comprises a DNase variant which compared to SEQ ID NO: 1 comprises at least two alterations selected from the group consisting of S26*, G37R, S69V, A76I, K86G,L,N,Q,T,V,Y, K95I, A101E, Q102E, K105N,G,Q,T,D, L129K, G141Q,R, V148I, P153D,V, K155E,F,L,S,T, Q157E, T159Q, A172E, V187N, Y, D197K,S, N214D and Y218D, E, wherein the variant has a sequence identity of at least 80%, but less than 100%, to the polypeptide of SEQ ID NO: 1, SEQ ID NO: 2 or SEQ ID NO: 3, and has DNase activity, and a detergent adjunct ingredient.

In one embodiment of the present invention the detergent composition comprises a DNase variant which compared to SEQ ID NO: 1 comprises at least one set of alterations shown in the below table and a detergent adjunct ingredient.

| | |
|---|---|
| 1 | N133Q, V148I, L180S |
| 2 | D46G, N133Q, K185* |
| 3 | K86L, K155L, A172H |
| 4 | K86T, K155S, E211T |
| 5 | K86T, K155E, A172R |
| 6 | K86L, T170Q, V187Y |
| 7 | K36H, K86T, K155E |
| 8 | A91R, A149E, K155T |
| 9 | K86Y, Q102E, A149E |
| 10 | A40H, K155S, V187N |
| 11 | K105Q, V187N, Y209H |
| 12 | A55I, K86V, K155T |
| 13 | S144E, A149E, K155E, K192A |
| 14 | K105D, N214D, Y218E |
| 15 | K147E, A172E, V187N, K192A |
| 16 | S69V, Q102E, T159Q |
| 17 | K36C, V138C |
| 18 | L92E, K147N, Q150D, Q157D |
| 19 | S69V, K86T, L92E, V187N |
| 20 | K82S, K155F, N214D |
| 21 | N133Q, N146A, P153D, D197S |
| 22 | S69V, G79C, P107C |
| 23 | K105G, F112Y, A172E |
| 24 | K86Q, N140H, K147E, K155F |
| 25 | F112W, N214D, Y218D |
| 26 | K105N, N140H, K147E, S154E |
| 27 | Q102E, N214D, Y218D |
| 28 | D32Q, K86V, K155E |
| 29 | F112W, N214D, Y218E |
| 30 | K86L, K95I, A149E |
| 31 | K86L, A101E, E211Y |
| 32 | A101E, N214D, Y218D |
| 33 | A101E, N214D, Y218E |
| 34 | A149E, N214D, Y218D |
| 35 | A149E, N214D, Y218E |
| 36 | A149E, P153D, A172E, Q208V |
| 37 | D46G, A101E, G141Q |
| 38 | F43W, K155F, N217A |
| 39 | K105G, N214D, Y218E |
| 40 | K105N, A149D, N213S |
| 41 | K105N, A172R, V187N |
| 42 | K105N, K155F, E211P |
| 43 | K105N, K155L, D197K |
| 44 | K105Q, K155F, N191* |
| 45 | K105Q, K155T, E211T |
| 46 | K105Q, V187N, K192A, D197S |
| 47 | K105T, K155S, V187N |
| 48 | K147N, A172D, V187N, K192A |
| 49 | K147N, Q150D, S154E, D197S |
| 50 | K155E, D197S, N217A |
| 51 | K36C, K105G, V138C |
| 52 | K82T, N140H, K155E |
| 53 | K86G, A101E, V187Y |
| 54 | K86G, K105Q, K155S |
| 55 | K86N, N146A, Q150D, S154E |
| 56 | K86Q, A101E, K105Q, N146A |
| 57 | K86Q, A149E, K155E, A172D |
| 58 | K86T, A101E, G199Q |
| 59 | K86V, A149F, K155F, A172E |
| 60 | K86V, K95I, K155E |
| 61 | K86V, K95I, K155L |
| 62 | K95I, A172D, V187N, K192A |
| 63 | K95I, S144E, A149F, S154E |
| 64 | N68D, N140H, K147N, K155E |
| 65 | P84D, K155L, K160D, A172D |
| 66 | P84D, N140H, K147E, Q158D |
| 67 | P84T, A101E, F112Y |
| 68 | P97E, N140H, A149E, K155L |
| 69 | Q102E, N146A, S154E, K160D |
| 70 | Q102E, N214D, Y218E |
| 71 | S144E, A149F, K155L, D197S |
| 72 | S26*, K105N, G141Q |
| 73 | S69V, K86Q, L92E, K192A |
| 74 | V148I, P153D, A172E, K192A |
| 75 | A101E, A172E, N214D, Y218E |
| 76 | A101E, K105N, N214D, Y218E |
| 77 | S69V, Q102E, P153V, T159Q |
| 78 | S69V, Q102E, Q157E, T159Q |

In one embodiment of the present invention the detergent adjunct ingredient is selected from the group consisting of surfactants, flocculating aids, chelating agents, dye transfer inhibitors, enzymes, enzyme stabilizers, enzyme inhibitors, catalytic materials, bleach activators, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, builders and co-builders, fabric hueing agents, anti-foaming agents, dispersants, processing aids, and/or pigments.

The detergent adjunct ingredient may be a surfactant. One advantage of including a surfactant in a detergent composition comprising a DNase variant is that the wash performance is improved. In one embodiment, the detergent adjunct ingredient is a builder or a clay soil removal/anti-redeposition agent. Detergent compositions will typically contain at least one surfactant as a detergent adjunct ingredient.

In one embodiment, detergent adjunct ingredient is an enzyme. The detergent composition may comprise one or more additional enzymes, as specified below. The one or more additional enzymes may be selected from the group consisting of proteases, lipases, cutinases, amylases, carbohydrases, cellulases, pectinases, mannanases, arabinases, galactanases, lichenase, xylanases and oxidases. Specific enzymes suitable for the detergent compositions of the present invention are described below.

In one embodiment, the detergent composition comprising a DNase variant of the present invention is capable of reducing adhesion of bacteria selected from the group consisting of Acinetobacter sp., Aeromicrobium sp., Brevundimonas sp., Microbacterium sp., Micrococcus luteus, Pseudomonas sp., Staphylococcus epidermidis, and Stenotrophomonas sp. to a surface, or releasing the bacteria from a surface to which they adhere. Biofilm growth in laundry items may originate from many organisms as described previously. One particular abundant bacterium in biofilm originates from Brevundimonas. The DNase variants of the present invention are particularly effective in reducing the growth of the bacterium and reducing the malodor, stickiness and re-deposition coursed by these bacteria. In one embodiment of the present invention, the surface is a textile surface. The textile can be made of cotton, cotton/polyester, polyester, polyamide, polyacryl and/or silk.

The DNase variants of the invention and detergent compositions containing the variants are suitable for use in cleaning, e.g. for use in laundry or hard surface cleaning such as dishwashing.

Thus, the present invention also relates to a method for cleaning an item, comprising exposing the item to a wash liquor comprising a DNase variant of the invention or to a detergent composition comprising a DNase variant of the invention. In one embodiment, the item to be cleaned may be a textile. In another embodiment, the item to be cleaned may be a hard surface, for example dishware, or e.g. a surface such as a tabletop, wall or floor or an interior surface of a machine such as a washing machine or a dishwasher.

One embodiment thus relates to a method for laundering an item, which method comprises the steps of:
a. exposing an item to a wash liquor comprising a DNase variant of the invention;
b. completing at least one wash cycle; and
C. optionally rinsing the item, wherein the item is a textile.

The pH of the liquid solution is in the range of 1 to 11, such as in the range of 5.5 to 11, such as in the range of 7 to 9, in the range of 7 to 8 or in the range of 7 to 8.5.

The wash liquor may have a temperature in the range of 5° C. to 95° C., or in the range of 10° C. to 80° C., in the range of 10° C. to 70° C., in the range of 10° C. to 60° C., in the range of 10° C. to 50° C., in the range of 15° C. to 40° C. or in the range of 20° C. to 30° C. In one embodiment the temperature of the wash liquor is 30° C.

In one embodiment of the present invention, the method for laundering an item further comprises draining of the wash liquor or part of the wash liquor after completion of a wash cycle. The wash liquor can then be re-used in a subsequent wash cycle or in a subsequent rinse cycle. The item may be exposed to the wash liquor during a first and optionally a second or a third wash cycle. In one embodiment the item is rinsed after being exposed to the wash liquor. The item can be rinsed with water or with water comprising a conditioner.

The DNase variants of the present invention may be added to a wash liquor as part of a detergent composition or separately. The concentration of the DNase variant in the wash liquor is typically in the range of from 0.0001 mg/l to 10 mg/l enzyme protein, from 0.0002 mg/l to 10 mg/l, from 0.001 mg/l to 10 mg/l, from 0.002 mg/l to 10 mg/l, from 0.01 mg/l to 10 mg/l, from 0.02 mg/l to 10 mg/l, from 0.1 mg/l to 10 mg/l, from 0.2 mg/l to 10 mg/l, or from 0.2 mg/l to 5 mg/l enzyme protein.

The amount of DNase in the cleaning composition may vary depending on factors such as the degree of concentration or compactness of the composition and the desired DNase concentration in the wash liquor. The DNase will normally be included in the cleaning composition in an amount of up to about 10,000 ppm, typically up to about 5000 ppm or up to about 2000 ppm. The DNase can e.g. be included in the cleaning composition at a level of from 1 ppm to 10,000 ppm, such as from 10 ppm to 5000 ppm, from 20 ppm to 2000 ppm, from 50 ppm to 1000 ppm, from 80 ppm to 600 ppm, or from 100 ppm to 500 ppm. The unit “ppm” in this context is intended to refer to mg/l for an enzyme added to a liquid composition (e.g. liquid, gel, etc.), or mg/kg for an enzyme added to a solid composition (e.g. powder, granulate, tablet, etc.)

The DNase variants of the invention may be stabilized using conventional stabilizing agents, e.g. a polyol such as propylene glycol or glycerol, a sugar or sugar alcohol, lactic acid, boric acid, or a boric acid derivative, e.g. an aromatic borate ester, or a phenyl boronic acid derivative such as 4-formylphenyl boronic acid, and the composition may be formulated as described in, for example, WO92/19709 and WO92/19708. The DNase variants of the invention may also be incorporated in the detergent compositions disclosed in WO97/07202, which is hereby incorporated by reference.

The composition of the present invention may be formulated as a bar, a homogenous tablet, and a tablet having two or more layers, a pouch having one or more compartments, a regular or compact powder, a granule, a paste, a gel, or a regular, compact or concentrated liquid. The detergent composition for example can be a liquid detergent, a powder detergent or a granule detergent.

In one embodiment, the DNase variant of the present invention is formulated in a liquid laundry composition comprising:
a) at least 0.005 mg of active DNase variant protein per mililitre detergent,
b) 2 wt % to 60 wt % of at least one surfactant, and
c) 5 wt % to 50 wt % of at least one builder.

In another embodiment of the present invention, the liquid laundry compositions composition may comprise:
a) at least 0.005 mg of DNase variant per mililitre of composition,
b) 1% to 15% by weight of at least one surfactant wherein the surfactant is LAS, AEOS and/or SLES, and
c) 5% to 50% by weight of at least one builder selected from HEDP, DTMPA or DTPMPA.

The liquid detergent composition may typically contain at least 20% by weight and up to 95% water, such as up to 70% water, up to 50% water, up to 40% water, up to 30% water, or up to 20% water. An aqueous liquid detergent may contain from 0-30% organic solvent. A liquid detergent may even be non-aqueous, wherein the water content is below 10%, preferably below 5%.

The detergent composition may also be formulated into a granular detergent for laundry or dishwashing. One embodiment of the present invention concerns a granular detergent composition comprising:
a) at least 0.005 mg of active DNase variant protein per gram of composition,
b) 5 wt % to 50 wt % anionic surfactant
c) 1 wt % to 8 wt % nonionic surfactant, and
d) 5 wt % to 40 wt % builder such as carbonates, zeolites, phosphate builder, calcium sequestering builders or complexing agents.

Any suitable detergent adjunct materials (e.g. surfactants or builders) known in the art may be used in the detergent composition of the present invention. Details and examples are described below.

### Additional Enzymes

The detergent composition of the present invention may comprise one or more additional enzymes such as a protease, lipase, cutinase, an amylase, carbohydrase, cellulase, pectinase, mannanase, arabinase, galactanase, lichenase, xylanase, nuclease, hexosaminidase, oxidase, e.g., a laccase, and/or peroxidase.

In general, the properties of the selected enzyme(s) should be compatible with the selected detergent, (i.e., pH-optimum, compatibility with other enzymatic and non-enzymatic ingredients, etc.), and the enzyme(s) should be present in effective amounts.

### Proteases

Suitable proteases may be of any origin, but are preferably of bacterial or fungal origin, optionally in the form of protein engineered or chemically modified mutants. The protease may be an alkaline protease, such as a serine protease or a metalloprotease. A serine protease may for example be of the S1 family, such as trypsin, or the S8 family such as a subtilisin. A metalloprotease may for example be a thermolysin, e.g. from the M4 family, or another metalloprotease such as those from the M5, M7 or M8 families.

The term “subtilases” refers to a sub-group of serine proteases according to Siezen et al., Protein Eng. 4 (1991) 719-737 and Siezen et al., Protein Sci. 6 (1997) 501-523. Serine proteases are a subgroup of proteases characterized by having a serine in the active site, which forms a covalent adduct with the substrate. The subtilases may be divided into six subdivisions, the Subtilisin family, the Thermitase family, the Proteinase K family, the Lantibiotic peptidase family, the Kexin family and the Pyrolysin family.

Although proteases suitable for detergent use may be obtained from a variety of organisms, including fungi such as Aspergillus, detergent proteases have generally been obtained from bacteria and in particular from Bacillus. Examples of Bacillus species from which subtilases have been derived include Bacillus lentus, Bacillus alkalophilus, Bacillus subtilis, Bacillus amyloliquefaciens, Bacillus licheniformis, Bacillus pumilus and Bacillus gibsonii. Particular subtilisins include subtilisin lentus, subtilisin Novo, subtilisin Carlsberg, subtilisin BPN′, subtilisin 309, subtilisin 147 and subtilisin 168 and e.g. protease PD138 (described in WO 93/18140). Other useful proteases are e.g. those described in WO 01/16285 and WO 02/16547.

Examples of trypsin-like proteases include the Fusarium protease described in WO 94/25583 and WO 2005/040372, and the chymotrypsin proteases derived from Cellumonas described in WO 2005/052161 and WO 2005/052146.

Examples of metalloproteases include the neutral metalloproteases described in WO 2007/044993 such as those derived from Bacillus amyloliquefaciens, as well as e.g. the metalloproteases described in WO 2015/158723 and WO 2016/075078.

Examples of useful proteases are the protease variants described in WO 89/06279 WO 92/19729, WO 96/34946, WO 98/20115, WO 98/20116, WO 99/11768, WO 01/44452, WO 03/006602, WO 2004/003186, WO 2004/041979, WO 2007/006305, WO 2011/036263, WO 2014/207227, WO 2016/087617 and WO 2016/174234. Preferred protease variants may, for example, comprise one or more of the mutations selected from the group consisting of: S3T, V41, S9R, S9E, A15T, S24G, S24R, K27R, N42R, S55P, G59E, G59D, N60D, N60E, V66A, N74D, S85R, A96S, S97G, S97D, S97A, S97SD, S99E, S99D, S99G, S99M, S99N, S99R, S99H, S101A, V1021, V102Y, V102N, S104A, G116V, G116R, H118D, H118N, A120S, S126L, P127Q, S128A, S154D, A156E, G157D, G157P, S158E, Y161A, R164S, Q176E, N179E, S182E, Q185N, A188P, G189E, V193M, N198D, V1991, Q200L, Y203W, S206G, L211Q, L211D, N212D, N212S, M216S, A226V, K229L, Q230H, Q239R, N246K, S253D, N255W, N255D, N255E, L256E, L256D T268A and R269H, wherein position numbers correspond to positions of the Bacillus lentus protease shown in SEQ ID NO: 1 of WO 2016/001449. Protease variants having one or more of these mutations are preferably variants of the Bacillus lentus protease (Savinase®, also known as subtilisin 309) shown in SEQ ID NO: 1 of WO 2016/001449 or of the Bacillus amyloliquefaciens protease (BPN′) shown in SEQ ID NO: 2 of WO 2016/001449. Such protease variants preferably have at least 80% sequence identity to SEQ ID NO: 1 or to SEQ ID NO: 2 of WO 2016/001449.

Another protease of interest is the alkaline protease from Bacillus lentus DSM 5483, as described for example in WO 91/02792, and variants thereof which are described for example in WO 92/21760, WO 95/23221, EP 1921147, EP 1921148 and WO 2016/096711.

The protease may alternatively be a variant of the TY145 protease having SEQ ID NO: 1 of WO 2004/067737, for example a variant comprising a substitution at one or more positions corresponding to positions 27, 109, 111, 171, 173, 174, 175, 180, 182, 184, 198, 199 and 297 of SEQ ID NO: 1 of WO 2004/067737, wherein said protease variant has a sequence identity of at least 75% but less than 100% to SEQ ID NO: 1 of WO 2004/067737. TY145 variants of interest are described in e.g. WO 2015/014790, WO 2015/014803, WO 2015/014804, WO 2016/097350, WO 2016/097352, WO 2016/097357 and WO 2016/097354.

Examples of preferred proteases include:
(a) variants of SEQ ID NO: 1 of WO 2016/001449 comprising two or more substitutions selected from the group consisting of S9E, N43R, N76D, Q206L, Y209W, S259D and L262E, for example a variant with the substitutions S9E, N43R, N76D, V2051, Q206L, Y209W, S259D, N261W and L262E, or with the substitutions S9E, N43R, N76D, N185E, S188E, Q191N, A194P, Q206L, Y209W, S259D and L262E, wherein position numbers are based on the numbering of SEQ ID NO: 2 of WO 2016/001449;
(b) a variant of the polypeptide of SEQ ID NO: 1 of WO 2016/001449 with the mutation S99SE, wherein position numbers are based on the numbering of SEQ ID NO: 2 of WO 2016/001449;
(c) a variant of the polypeptide of SEQ ID NO: 1 of WO 2016/001449 with the mutation S99AD, wherein position numbers are based on the numbering of SEQ ID NO: 2 of WO 2016/001449;
(d) a variant of the polypeptide of SEQ ID NO: 1 of WO 2016/001449 with the substitutions Y167A+R170S+A194P, wherein position numbers are based on the numbering of SEQ ID NO: 2 of WO 2016/001449;
(e) a variant of the polypeptide of SEQ ID NO: 1 of WO 2016/001449 with the substitutions S9R+A15T+V68A+N218D+Q245R, wherein position numbers are based on the numbering of SEQ ID NO: 2 of WO 2016/001449;
(f) a variant of the polypeptide of SEQ ID NO: 1 of WO 2016/001449 with the substitutions S9R+A15T+G61E+V68A+A194P+V2051+Q245R+N261D, wherein position numbers are based on the numbering of SEQ ID NO: 2 of WO 2016/001449;
(g) a variant of the polypeptide of SEQ ID NO: 1 of WO 2016/001449 with the substitutions S99D+S101R/E+S103A+V104I+G160S; for example a variant of SEQ ID NO: 1 of WO 2016/001449 with the substitutions S3T+V41+S99D+S101E+S103A+V104I+G160S+V2051, wherein position numbers are based on the numbering of SEQ ID NO: 2 of WO 2016/001449;
(h) a variant of the polypeptide of SEQ ID NO: 2 of WO 2016/001449 with the substitutions S24G+S53G+S78N+S101N+G128A/S+Y217Q, wherein position numbers are based on the numbering of SEQ ID NO: 2 of WO 2016/001449;
(i) the polypeptide disclosed in GENESEQP under accession number BER84782, corresponding to SEQ ID NO: 302 in WO 2017/210295;
(j) a variant of the polypeptide of SEQ ID NO: 1 of WO 2016/001449 with the substitutions S99D+S101E+S103A+V104I+S156D+G160S+L262E, wherein position numbers are based on the numbering of SEQ ID NO: 2 of WO 2016/001449;
(k) a variant of the polypeptide of SEQ ID NO: 1 of WO 2016/001449 with the substitutions S9R+A15T+G61E+V68A+N76D+S99G+N218D+Q245R, wherein position numbers are based on the numbering of SEQ ID NO: 2 of WO 2016/001449;
(l) a variant of the polypeptide of SEQ ID NO: 1 of WO 2016/001449 with the substitutions V68A+S106A, wherein position numbers are based on the numbering of SEQ ID NO: 2 of WO 2016/001449; and
(m) a variant of the polypeptide of SEQ ID NO: 1 of WO 2004/067737 with the substitutions S27K+N109K+S111E+S171E+S173P+G174K+S175P+F180Y+G182A+L184F+Q198E+N199+T297P, wherein position numbers are based on the numbering of SEQ ID NO: 1 of WO 2004/067737.

Suitable commercially available protease enzymes include those sold under the trade names Alcalase®, Duralase™, Durazym™, Relase®, Relase® Ultra, Savinase®, Savinase® Ultra, Primase™, Polarzyme®, Kannase®, Liquanase®, Liquanase® Ultra, Ovozyme®, Coronase®, Coronase® Ultra, Blaze®, Blaze® Pro, Blaze Evity® 100T, Blaze Evity® 125T, Blaze Evity® 150T, Blaze Evity® 200T, Neutrase®, Everlase®, Esperase®, Progress® Uno, Progress® In and Progress® Excel (Novozymes A/S), those sold under the tradename Maxatase™, Maxacal™, Maxapem®, Purafect® Ox, Purafect® OxP, Puramax®, FN2™, FN3™, FN4^{ex}™, Excellase®, Excellenz™ P1000, Excellenz™ P1250, Eraser™, Preferenz® P100, Preferenz® P300, Purafect Prime, Preferenz P110™, Effectenz P1000™, Purafect®, Effectenz P1050™ Purafect® Ox, Effectenz™ P2000, Purafast™, Properase®, Opticlean™ and Optimase® (Danisco/DuPont), BLAP (sequence shown in FIG. 29 of U.S. Pat. No. 5,352,604) and variants hereof (Henkel AG), and KAP (Bacillus alkalophilus subtilisin) from Kao.

### Lipases and Cutinases

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

### Amylases

Suitable amylases which can be used together with the DNase of the invention may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:
M197T;
H156Y+A181T+N190F+A209V+Q264S; or
G48A+T49I+G107A+H156Y+A181T+N190F+I201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, I206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:
N128C+K178L+T182G+Y305R+G475K;
N128C+K178L+T182G+F202Y+Y305R+D319T+G475K;
S125A+N128C+K178L+T182G+Y305R+G475K; or
S125A+N128C+T131I+T165I+K178L+T182G+Y305R+G475K wherein the variants are C-terminally truncated and optionally further comprises a substitution at position 243 and/or a deletion at position 180 and/or position 181.

Further suitable amylases are amylases having SEQ ID NO: 1 of WO13184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, I203, S241, R458, T459, D460, G476 and G477. More preferred variants of SEQ ID NO: 1 are those having the substitution in one of more of the following positions: K176L, E187P, N192FYH, M199L, I203YF, S241QADN, R458N, T459S, D460T, G476K and G477K and/or deletion in position R178 and/or S179 or of T180 and/or G181. Most preferred amylase variants of SEQ ID NO: 1 are those having the substitutions:
E187P+I203Y+G476K
E187P+I203Y+R458N+T459S+D460T+G476K
   wherein the variants optionally further comprise a substitution at position 241 and/or a deletion at position 178 and/or position 179.

Further suitable amylases are amylases having SEQ ID NO: 1 of WO10104675 or variants having 90% sequence identity to SEQ ID NO: 1 thereof. Preferred variants of SEQ ID NO: 1 are those having a substitution, a deletion or an insertion in one of more of the following positions: N21, D97, V128 K177, R179, S180, I181, G182, M200, L204, E242, G477 and G478. More preferred variants of SEQ ID NO: 1 are those having the substitution in one of more of the following positions: N21D, D97N, V128I K177L, M200L, L204YF, E242QA, G477K and G478K and/or deletion in position R179 and/or S180 or of I181 and/or G182. Most preferred amylase variants of SEQ ID NO: 1 are those having the substitutions:
N21D+D97N+V128I
   wherein the variants optionally further comprise a substitution at position 200 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Fungamyl™, Stainzyme™, Stainzyme Plus™, Natalase™, Liquozyme X and BAN™ (from Novozymes A/S), and Rapidase™, Purastar™/Effectenz™, Powerase, Preferenz S1000, Preferenz S100 and Preferenz S110 (from Genencor International Inc./DuPont).

### Cellulases

Suitable cellulases include mono-component and mixtures of enzymes of bacterial or fungal origin. Chemically modified or protein engineered mutants are also contemplated. The cellulase may for example be a mono-component or a mixture of mono-component endo-1,4-beta-glucanase also referred to as endoglucanase.

Suitable cellulases include those from the genera Bacillus, Pseudomonas, Humicola, Myceliophthora, Fusarium, Thielavia, Trichoderma, and Acremonium. Exemplary cellulases include a fungal cellulase from Humicola insolens (U.S. Pat. No. 4,435,307) or from Trichoderma, e.g. T. reesei or T. viride. Other suitable cellulases are from Thielavia e.g. Thielavia terrestris as described in WO 96/29397 or the fungal cellulases produced from Myceliophthora thermophila and Fusarium oxysporum disclosed in U.S. Pat. Nos. 5,648,263, 5,691,178, 5,776,757, WO 89/09259 and WO 91/17244. Also relevant are cellulases from Bacillus as described in WO 02/099091 and JP 2000210081. Suitable cellulases are alkaline or neutral cellulases having care benefits. Examples of cellulases are described in EP 0 495 257, EP 0 531 372, WO 96/11262, WO 96/29397, WO 98/08940. Other examples are cellulase variants such as those described in WO 94/07998, EP 0 531 315, U.S. Pat. Nos. 5,457,046, 5,686,593, 5,763,254, WO 95/24471, WO 98/12307.

Other cellulases are endo-beta-1,4-glucanase enzyme having a sequence of at least 97% identity to the amino acid sequence of position 1 to position 773 of SEQ ID NO:2 of WO 2002/099091 or a family 44 xyloglucanase, which a xyloglucanase enzyme having a sequence of at least 60% identity to positions 40-559 of SEQ ID NO: 2 of WO 2001/062903.

Commercially available cellulases include Carezyme®, Carezyme® Premium, Celluzyme®, Celluclean®, Celluclast®, Endolase®, Renozyme®; Whitezyme® Celluclean® Classic, Cellusoft® (Novozymes A/S), Puradax®, Puradax HA, and Puradax EG (available from Genencor International Inc.) and KAC-500(B)™ (Kao Corporation).

### Mannanases

Suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 1999/064619. A commercially available mannanase is Mannaway (Novozymes A/S).

### Hexosaminidases

Detergent compositions comprising a DNase of the invention may also include one or more hexosaminidases. The term hexosaminidase includes “dispersin” and the abbreviation “Dsp”, which means a polypeptide having hexosaminidase activity, EC 3.2.1.-, that catalyzes the hydrolysis of β-1,6-glycosidic linkages of N-acetyl-glucosamine polymers found e.g. in biofilm. The term hexosaminidase includes polypeptides having N-acetylglucosaminidase activity and β-N-acetylglucosaminidase activity.

A polypeptide having hexosaminidase activity may be obtained from microorganisms of any genus, in particular from bacteria or fungi. Preferably the hexosaminidase, e.g. a dispersin, is obtained from Terribacillus, Curtobacterium, Aggregatibacter, Haemophilus or Actinobacillus, preferably Terribacillus. The hexosaminidase may also be a variant of a polypeptide obtained from any of these or other organisms.

Suitable hexosaminidases include those disclosed in WO2017186936, WO2017186937, WO2017186943, WO2017207770, WO2018184873, WO2019086520, WO2019086528, WO2019086530, WO2019086532, WO2019086521, WO2019086526, WO2020002604, WO2020002608, WO2020007863, WO2020007875, WO2020008024, WO2020070063, WO2020070249, WO2020088957, WO2020088958 and WO2020207944.

### Peroxidases/Oxidases

Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257. Commercially available peroxidases include Guardzyme™ (Novozymes A/S).

A suitable peroxidase is preferably a peroxidase enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity.

Suitable peroxidases also include a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions. The haloperoxidase may be a chloroperoxidase. Preferably, the haloperoxidase is a vanadium haloperoxidase, i.e., a vanadate-containing haloperoxidase. In a preferred method the vanadate-containing haloperoxidase is combined with a source of chloride ion.

Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis.

Haloperoxidases have also been isolated from bacteria such as Pseudomonas, e.g., P. pyrrocinia and Streptomyces, e.g., S. aureofaciens.

The haloperoxidase may be derivable from Curvularia sp., in particular Curvularia verruculosa or Curvularia inaequalis, such as C. inaequalis CBS 102.42 as described in WO 95/27046; or C. verruculosa CBS 147.63 or C. verruculosa CBS 444.70 as described in WO 97/04102; or from Drechslera hartlebii as described in WO 01/79459, Dendryphiella salina as described in WO 01/79458, Phaeotrichoconis crotalarie as described in WO 01/79461, or Geniculosporium sp. as described in WO 01/79460.

Suitable oxidases include, in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5).

Preferred laccase enzymes are enzymes of microbial origin. The enzymes may be derived from plants, bacteria or fungi (including filamentous fungi and yeasts).

Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporous, e.g., P. pinsitus, Phlebia, e.g., P. radiata (WO 92/01046), or Coriolus, e.g., C. hirsutus (JP 2238885).

Suitable examples from bacteria include a laccase derivable from a strain of Bacillus. A laccase derived from Coprinopsis or Myceliophthora is preferred; in particular a laccase derived from Coprinopsis cinerea, as disclosed in WO 97/08325; or from Myceliophthora thermophila, as disclosed in WO 95/33836.

### Protease Stabilizers/Inhibitors

The protease(s), as described above, may be stabilized using compounds that act by temporarily reducing the proteolytic activity (reversible inhibitors).

Thus, the composition of the invention may also include a protease inhibitor/stabilizer, which is a reversible inhibitor of protease activity, e.g., serine protease activity. Preferably, the protease inhibitor is a (reversible) subtilisin protease inhibitor. In particular, the protease inhibitor may be a peptide aldehyde, boric acid, or a boronic acid; or a derivative of any of these.

### Boronic Acids

The protease inhibitor may be a boronic acid or a derivative thereof; preferably, a phenylboronic acid or a derivative thereof. In an embodiment of the invention, the phenyl boronic acid derivative is of the following formula:

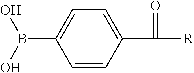

wherein R is selected from the group consisting of hydrogen, hydroxy, C1-C6 alkyl, substituted C1-C6 alkyl, C1-C6 alkenyl and substituted C1-C6 alkenyl. Preferably, R is hydrogen, CH₃, CH₃CH₂ or CH₃CH₂CH₂.

In a preferred embodiment, the protease inhibitor (phenyl boronic acid derivative) is 4-formyl-phenyl boronic acid (4-FPBA).

In another particular embodiment, the protease inhibitor is selected from the group consisting of thiophene-2 boronic acid, thiophene-3 boronic acid, acetamidophenyl boronic acid, benzofuran-2 boronic acid, naphtalene-1 boronic acid, naphtalene-2 boronic acid, 2-FPBA, 3-FBPA, 4-FPBA, 1-thianthrene boronic acid, 4-dibenzofuran boronic acid, 5-methylthiophene-2 boronic, acid, thionaphthene boronic acid, furan-2 boronic acid, furan-3 boronic acid, 4,4 biphenyl-diborinic acid, 6-hydroxy-2-naphtalene, 4-(methylthio) phenyl boronic acid, 4 (trimethyl-silyl)phenyl boronic acid, 3-bromothiophene boronic acid, 4-methylthiophene boronic acid, 2-naphtyl boronic acid, 5-bromothiophene boronic acid, 5-chlorothiophene boronic acid, dimethylthiophene boronic acid, 2-bromophenyl boronic acid, 3-chlorophenyl boronic acid, 3-methoxy-2-thiophene, p-methyl-phenylethyl boronic acid, 2-thianthrene boronic acid, di-benzothiophene boronic acid, 4-carboxyphenyl boronic acid, 9-anthryl boronic acid, 3,5 dichlorophenyl boronic, acid, diphenyl boronic acidanhydride, o-chlorophenyl boronic acid, p-chlorophenyl boronic acid, m-bromophenyl boronic acid, p-bromophenyl boronic acid, p-fluorophenyl boronic acid, p-tolyl boronic acid, o-tolyl boronic acid, octyl boronic acid, 1,3,5 trimethylphenyl boronic acid, 3-chloro-4-fluorophenyl boronic acid, 3-aminophenyl boronic acid, 3,5-bis-(trifluoromethyl) phenyl boronic acid, 2,4 dichlorophenyl boronic acid, and 4-methoxyphenyl boronic acid.

Further boronic acid derivatives suitable as protease inhibitors in the detergent composition are described in U.S. Pat. Nos. 4,963,655, 5,159,060, WO 95/12655, WO 95/29223, WO 92/19707, WO 94/04653, WO 94/04654, U.S. Pat. Nos. 5,442,100, 5,488,157 and 5,472,628.

### Peptide Aldehyde or Ketone

The protease stabilizer may have the formula: P-A-L-B-B0-R* wherein:
R* is H (hydrogen), CH₃, CX₃, CHX₂, or CH₂X, wherein X is a halogen atom, particularly F (fluorine); preferably, R*═H (so the stabilizer is a peptide aldehyde with the formula P-A-L-B-B0-H);
L is either absent or a linker group of the formula —C(═O)—, —C(═O)—C(═O)—, —C(═S)—, —C(═S)—C(═S)—, or —C(═S)—C(═O)—;
A is absent if L is absent, or is 1 or 2 amino acid residues connected to L via the N-terminal; thus, A may represent A1 or A2-A1, where A2 and A1 each represent one amino acid residue;
B may be 1, 2, or 3 amino acid residues; thus, B may represent B1, B2-B1, or B3-B2-B1, which is connected to B0 via the C-terminal, where B3, B2, and B1 each represent one amino acid residue; B0 is a single amino acid residue with L- or D-configuration of the formula —NH—CH(R)—C(═O)—;
R is independently selected from the group consisting of C₁₋₆ alkyl, C₆₋₁₀ aryl or C₇₋₁₀ arylalkyl, optionally substituted with one or more, identical or different, substituents R′;
R′ is independently selected from the group consisting of halogen, —OH, —OR″, —SH, —SR″, —NH₂, —NHR″, —NR″₂, —CO₂H, —CONH₂, —CONHR″, —CONR″₂, —NHC(═N)NH₂;
R″ is a C₁₋₆ alkyl group; and
P is selected from the group consisting of hydrogen, or—if L is absent—an N-terminal protection group;
B0 may be a single amino acid residue with L- or D-configuration, which is connected to H via the C-terminal of the amino acid. B0 has the formula —NH—CH(R)—C(═O)—, wherein R is a C₁₋₆ alkyl, C₆₋₁₀ aryl or C₇₋₁₀ arylalkyl side chain, such as methyl, ethyl, propyl, isopropyl, butyl, isobutyl, phenyl or benzyl, and wherein R may be optionally substituted with one or more, identical or different, substituents R′. Particular examples of B0 are the D- or L-form of arginine (Arg), 3,4-dihydroxyphenylalanine, isoleucine (Ile), leucine (Leu), methionine (Met), norleucine (Nle), norvaline (Nva), phenylalanine (Phe), m-tyrosine, p-tyrosine (Tyr) and valine (Val). A particular embodiment is when B0 is leucine, methionine, phenylalanine, p-tyrosine, or valine. Particularly preferred is p-tyrosine.
B1, which is connected to B0 via the C-terminal of the amino acid, may be an aliphatic, hydrophobic and/or neutral amino acid. Examples of B1 are alanine (Ala), cysteine (Cys), glycine (Gly), isoleucine (Ile), leucine (Leu), norleucine (Nle), norvaline (Nva), proline (Pro), serine (Ser), threonine (Thr) and valine (Val). Particular examples of B1 are alanine, glycine, isoleucine, leucine and valine. A particular embodiment is when B1 is alanine, glycine, or valine.
B2, if present, is connected to B1 via the C-terminal of the amino acid, and may be an aliphatic, hydrophobic, neutral and/or polar amino acid. Examples of B2 are alanine (Ala), arginine (Arg), capreomycidine (Cpd), cysteine (Cys), glycine (Gly), isoleucine (Ile), leucine (Leu), norleucine (Nle), norvaline (Nva), phenylalanine (Phe), proline (Pro), serine (Ser), threonine (Thr), and valine (Val). Particular examples of B2 are alanine, arginine, capreomycidine, glycine, isoleucine, leucine, phenylalanine and valine. A particular embodiment is when B2 is arginine, glycine, leucine, phenylalanine, or valine.
B3, if present, is connected to B2 via the C-terminal of the amino acid, and may be a large, aliphatic, aromatic, hydrophobic and/or neutral amino acid. Examples of B3 are isoleucine (Ile), leucine (Leu), norleucine (Nle), norvaline (Nva), phenylalanine (Phe), phenylglycine, tyrosine (Tyr), tryptophan (Trp) and valine (Val). Particular examples of B3 are leucine, phenylalanine, tyrosine, and tryptophan.

The linker group L may be absent or selected from the group consisting of —C(═O)—, —C(═O)—C(═O)—, —C(═S)—, —C(═S)—C(═S)— or —C(═S)—C(═O)—. Particular embodiments of the invention are when L is absent or L is a carbonyl group-C(═O)—.

A1, if present, is connected to L via the N-terminal of the amino acid, and may be an aliphatic, aromatic, hydrophobic, neutral and/or polar amino acid. Examples of A1 are alanine (Ala), arginine (Arg), capreomycidine (Cpd), glycine (Gly), isoleucine (Ile), leucine (Leu), norleucine (Nle), norvaline (Nva), phenylalanine (Phe), threonine (Thr), tyrosine (Tyr), tryptophan (Trp) and valine (Val). Particular examples of A1 are alanine, arginine, glycine, leucine, phenylalanine, tyrosine, tryptophan and valine. A particular embodiment is when B2 is leucine, phenylalanine, tyrosine or tryptophan.

A2, if present, is connected to A1 via the N-terminal of the amino acid, and may be a large, aliphatic, aromatic, hydrophobic and/or neutral amino acid. Examples of A2 are arginine (Arg), isoleucine (Ile), leucine (Leu), norleucine (Nle), norvaline (Nva), phenylalanine (Phe), phenylglycine, Tyrosine (Tyr), tryptophan (Trp) and valine (Val). Particular examples of A2 are phenylalanine and tyrosine.

The N-terminal protection group P (if present) may be selected from formyl, acetyl (Ac), benzoyl (Bz), trifluoroacetyl, methoxysuccinyl, aromatic and aliphatic urethane protecting groups such as fluorenylmethyloxycarbonyl (Fmoc), methoxycarbonyl (Moc), (fluoromethoxy)carbonyl, benzyloxycarbonyl (Cbz), t-butyloxycarbonyl (Boc) and adamantyloxycarbonyl; p-methoxybenzyl carbonyl, benzyl (Bn), p-methoxybenzyl (PMB), p-methoxyphenyl (PMP), methoxyacetyl, methylamino carbonyl, methylsulfonyl, ethylsulfonyl, benzylsulfonyl, methylphosphoramidyl (MeOP(OH)(═O)) and benzylphosphoramidyl (PhCH₂OP(OH)(═O)).

Suitable peptide aldehydes are described in WO94/04651, WO95/25791, WO98/13458, WO98/13459, WO98/13460, WO98/13461, WO98/13462, WO07/141736, WO07/145963, WO09/118375, WO10/055052 and WO11/036153. More particularly, the peptide aldehyde may be Cbz-Arg-Ala-Tyr-H, Ac-Gly-Ala-Tyr-H, Cbz-Gly-Ala-Tyr-H, Cbz-Gly-Ala-Tyr-CF₃, Cbz-Gly-Ala-Leu-H, Cbz-Val-Ala-Leu-H, Cbz-Val-Ala-Leu-CF₃, Moc-Val-Ala-Leu-CF₃, Cbz-Gly-Ala-Phe-H, Cbz-Gly-Ala-Phe-CF₃, Cbz-Gly-Ala-Val-H, Cbz-Gly-Gly-Tyr-H, Cbz-Gly-Gly-Phe-H, Cbz-Arg-Val-Tyr-H, Cbz-Leu-Val-Tyr-H, Ac-Leu-Gly-Ala-Tyr-H, Ac-Phe-Gly-Ala-Tyr-H, Ac-Tyr-Gly-Ala-Tyr-H, Ac-Phe-Gly-Ala-Leu-H, Ac-Phe-Gly-Ala-Phe-H, Ac-Phe-Gly-Val-Tyr-H, Ac-Phe-Gly-Ala-Met-H, Ac-Trp-Leu-Val-Tyr-H, MeO—CO-Val-Ala-Leu-H, MeNCO-Val-Ala-Leu-H, MeO—CO-Phe-Gly-Ala-Leu-H, MeO—CO-Phe-Gly-Ala-Phe-H, MeSO₂-Phe-Gly-Ala-Leu-H, MeSO₂-Val-Ala-Leu-H, PhCH₂O—P(OH)(O)-Val-Ala-Leu-H, EtSO₂-Phe-Gly-Ala-Leu-H, PhCH₂SO₂-Val-Ala-Leu-H, PhCH₂O—P(OH)(O)-Leu-Ala-Leu-H, PhCH₂O—P(OH)(O)-Phe-Ala-Leu-H, or MeO—P(OH)(O)-Leu-Gly-Ala-Leu-H. A preferred stabilizer for use in the liquid composition of the invention is Cbz-Gly-Ala-Tyr-H, or a hydrosulfite adduct thereof, wherein Cbz is benzyloxycarbonyl.

Further examples of such peptide aldehydes include a-MAPI, B-MAPI, Phe-C(═O)-Arg-Val-Tyr-H, Phe-C(═O)-Gly-Gly-Tyr-H, Phe-C(═O)-Gly-Ala-Phe-H, Phe-C(═O)-Gly-Ala-Tyr-H, Phe-C(═O)-Gly-Ala-L-H, Phe-C(═O)-Gly-Ala-Nva-H, Phe-C(═O)-Gly-Ala-Nle-H, Tyr-C(═O)-Arg-Val-Tyr-H, Tyr-C(═O)-Gly-Ala-Tyr-H, Phe-C(═S)-Arg-Val-Phe-H, Phe-C(═S)-Arg-Val-Tyr-H, Phe-C(═S)-Gly-Ala-Tyr-H, Antipain, GE20372A, GE20372B, Chymostatin A, Chymostatin B, and Chymostatin C.

The protease stabilizer may be a hydrosulfite adduct of the peptide aldehyde or ketone described above, e.g., as described in WO 2013/004636. The adduct may have the formula P-A-L-B—N(H)—CHR—CH(OH)—SO₃M, wherein P, A, L, B, and R are defined as above, and M is H or an alkali metal, preferably Na or K.

An aqueous solution of the hydrosulfite adduct may be prepared by reacting the corresponding peptide aldehyde with an aqueous solution of sodium bisulfite (sodium hydrogen sulfite, NaHSO₃); potassium bisulfite (KHSO₃) by known methods, e.g., as described in WO 98/47523; U.S. Pat. Nos. 6,500,802; 5,436,229; J. Am. Chem. Soc. (1978) 100, 1228; Org. Synth., Coll. vol. 7: 361.

Particularly preferred peptide aldehyde protease stabilizers have the formula P-B3-B2-B1-B0-H, or a hydrosulfite adduct having the formula P-B3-B2-B1-N(H)—CHR—CHOH—SO₃M, wherein
i) H is hydrogen;
ii) B0 is a single amino acid residue with L- or D-configuration of the formula —NH—CH(R)—C(═O)—;
iii) B1 and B2 are independently single amino acid residues;
iv) B3 is a single amino acid residue, or is absent;
v) R is independently selected from the group consisting of C₁₋₆ alkyl, C₆₋₁₀ aryl or C₇₋₁₀ arylalkyl optionally substituted with one or more, identical or different, substituents R′;
vi) R′ is independently selected from the group consisting of halogen, —OH, —OR″, —SH, —SR″, —NH₂, —NHR″, —NR″₂, —CO₂H, —CONH₂, —CONHR″, —CONR″₂, —NHC(═N)NH₂;
vii) R″ is a C₁₋₆ alkyl group;
viii) P is an N-terminal protection group, preferably methoxycarbonyl (Moc) or benzyloxycarbonyl (Cbz); and
ix) M is H or an alkali metal, preferably Na or K.

In an even more preferred embodiment, the peptide aldehyde protease stabilizer has the formula P-B2-B1-B0-H or an adduct having the formula P-B2-B1-N(H)—CHR—CHOH—SO₃M, wherein
i) H is hydrogen;
ii) B0 is a single amino acid residue with L- or D-configuration of the formula —NH—CH(R)—C(═O)—;
iii) B1 and B2 are independently single amino acid residues;
iv) R is independently selected from the group consisting of C₁₋₆ alkyl, C₆₋₁₀ aryl or C₇₋₁₀ arylalkyl optionally substituted with one or more, identical or different, substituents R′;
v) R′ is independently selected from the group consisting of halogen, —OH, —OR″, —SH, —SR″, —NH₂, —NHR″, —NR″₂, —CO₂H, —CONH₂, —CONHR″, —CONR″₂, —NHC(═N)NH₂;
vi) R″ is a C₁₋₆ alkyl group;
vii) P is an N-terminal protection group, preferably methoxycarbonyl (Moc) or benzyloxycarbonyl (Cbz); and
viii) M is H or an alkali metal, preferably Na or K.

Preferred embodiments of B0, B1, B2, B3, and P are as described above.

When the peptide aldehyde has the formula P-B3-B2-B1-B0-H, or a hydrosulfite adduct thereof, P is preferably acetyl, methoxycarbonyl, benzyloxycarbonyl, methylamino carbonyl, methylsulfonyl, benzylsulfonyl and benzylphosphoramidyl.

When the peptide aldehyde has the formula P-B2-B1-B0-H, or a hydrosulfite adduct thereof, P is preferably acetyl, methoxycarbonyl, methylsulfonyl, ethylsulfonyl and methylphosphoramidyl.

The molar ratio of the above-mentioned peptide aldehydes (or hydrosulfite adducts) to the protease may be at least 1:1 or 1.5:1, and it may be less than 1000:1, more preferred less than 500:1, even more preferred from 100:1 to 2:1 or from 20:1 to 2:1, or most preferred, the molar ratio is from 10:1 to 2:1.

Formate salts (e.g., sodium formate) and formic acid have also shown good effects as inhibitor of protease activity. Formate can be used synergistically with the above-mentioned protease inhibitors, as shown in WO 2013/004635. The formate salts may be present in the composition in an amount of at least 0.1% w/w or 0.5% w/w, e.g., at least 1.0%, at least 1.2% or at least 1.5%. The amount is typically below 5% w/w, below 4% or below 3%.

In an embodiment, the protease is a metalloprotease and the inhibitor is a metalloprotease inhibitor, e.g., a protein hydrolysate based inhibitor (e.g., as described in WO 2008/134343).

### Surfactants

The detergent composition may comprise one or more surfactants, which may be anionic and/or cationic and/or non-ionic and/or semi-polar and/or zwitterionic, or a mixture thereof. In a particular embodiment, the detergent composition includes a surfactant system (comprising more than one surfactant) e.g. a mixture of one or more nonionic surfactants and one or more anionic surfactants. In one embodiment the detergent comprises at least one anionic surfactant than at least one non-ionic surfactant, the weight ratio of anionic to nonionic surfactant may be from 10:1 to 1:10. In one embodiment the amount of anionic surfactant is higher than the amount of non-ionic surfactant e.g. the weight ratio of anionic to non-ionic surfactant may be from 10:1 to 1.1:1 or from 5:1 to 1.5:1. The amount of anionic to non-ionic surfactant may also be equal and the weight ratios 1:1. In one embodiment the amount of non-ionic surfactant is higher than the amount of anionic surfactant and the weight ratio may be 1:10 to 1:1.1. Preferably the weight ratio of anionic to non-ionic surfactant is from 10:1 to 1:10, such as from 5:1 to 1:5, or from 5:1 to 1:1.2. Preferably, the weight fraction of non-ionic surfactant to anionic surfactant is from 0 to 0.5 or 0 to 0.2 thus non-ionic surfactant can be present or absent if the weight fraction is 0, but if non-ionic surfactant is present, then the weight fraction of the nonionic surfactant is preferably at most 50% or at most 20% of the total weight of anionic surfactant and non-ionic surfactant. Light duty detergent usually comprises more nonionic than anionic surfactant and there the fraction of non-ionic surfactant to anionic surfactant is preferably from 0.5 to 0.9. The total weight of surfactant(s) is typically present at a level of from about 0.1% to about 60% by weight, such as about 1% to about 40%, or about 3% to about 20%, or about 3% to about 10%.

The surfactant(s) is chosen based on the desired cleaning application, and may include any conventional surfactant(s) known in the art. When included therein the detergent will usually contain from about 1% to about 40% by weight of an anionic surfactant, such as from about 5% to about 30%, including from about 5% to about 15%, or from about 15% to about 20%, or from about 20% to about 25% of an anionic surfactant. Non-limiting examples of anionic surfactants include sulfates and sulfonates, typically available as sodium or potassium salts or salts of monoethanolamine (MEA, 2-aminoethan-1-ol) or triethanolamine (TEA, 2,2′,2″-nitrilotriethan-1-ol); in particular, linear alkylbenzenesulfonates (LAS), isomers of LAS such as branched alkylbenzenesulfonates (BABS) and phenylalkanesulfonates; olefin sulfonates, in particular alpha-olefinsulfonates (AOS); alkyl sulfates (AS), in particular fatty alcohol sulfates (FAS), i.e., primary alcohol sulfates (PAS) such as dodecyl sulfate; alcohol ethersulfates (AES or AEOS or FES, also known as alcohol ethoxysulfates or fatty alcohol ether sulfates); paraffin sulfonates (PS) including alkane-1-sulfonates and secondary alkanesulfonates (SAS); ester sulfonates, including sulfonated fatty acid glycerol esters and alpha-sulfo fatty acid methyl esters (alpha-SFMe or SES or MES); alkyl- or alkenylsuccinic acids such as dodecenyl/tetradecenyl succinic acid (DTSA); diesters and monoesters of sulfosuccinic acid; fatty acid derivatives of amino acids. Furthermore, salts of fatty acids (soaps) may be included.

When included therein the detergent will usually contain from about 1% to about 40% by weight of a cationic surfactant, for example from about 0.5% to about 30%, in particular from about 1% to about 20%, from about 3% to about 10%, such as from about 3% to about 5%, from about 8% to about 12% or from about 10% to about 12%. Non-limiting examples of cationic surfactants include alkyldimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, ester quats, and combinations thereof.

When included therein the detergent will usually contain from about 0.2% to about 40% by weight of a nonionic surfactant, for example from about 0.5% to about 30%, in particular from about 1% to about 20%, from about 3% to about 10%, such as from about 3% to about 5%, from about 8% to about 12%, or from about 10% to about 12%. Non-limiting examples of nonionic surfactants include alcohol ethoxylates (AE or AEO) e.g. the AEO-series such as AEO-7, alcohol propoxylates, in particular propoxylated fatty alcohols (PFA), ethoxylated and propoxylated alcohols, alkoxylated fatty acid alkyl esters, such as ethoxylated and/or propoxylated fatty acid alkyl esters (in particular methyl ester ethoxylates, MEE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof.

When included therein the detergent will usually contain from about 0.01 to about 10% by weight of a semipolar surfactant. Non-limiting examples of semipolar surfactants include amine oxides (AO) such as alkyldimethylamine oxides, in particular N-(coco alkyl)-N, N-dimethylamine oxide and N-(tallow-alkyl)-N, N-bis(2-hydroxyethyl)amine oxide, and combinations thereof.

When included therein the detergent will usually contain from about 0.01% to about 10% by weight of a zwitterionic surfactant. Non-limiting examples of zwitterionic surfactants include betaines such as alkyldimethylbetaines, sulfobetaines, and combinations thereof.

Additional bio-based surfactants may be used e.g. wherein the surfactant is a sugar-based non-ionic surfactant which may be a hexyl-β-D-maltopyranoside, thiomaltopyranoside or a cyclic-maltopyranoside, such as described in EP2516606 B1.

### Hydrotropes

A hydrotrope is a compound that solubilizes hydrophobic compounds in aqueous solutions (or oppositely, polar substances in a non-polar environment). Typically, hydrotropes have both hydrophilic and a hydrophobic character (so-called amphiphilic properties as known from surfactants); however the molecular structure of hydrotropes generally do not favor spontaneous self-aggregation, see e.g. review by Hodgdon and Kaler (2007), Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in detergent compositions allow for example more concentrated formulations of surfactants (as in the process of compacting liquid detergents by removing water) without inducing undesired phenomena such as phase separation or high viscosity.

The detergent may contain 0-10% by weight, for example 0-5% by weight, such as about 0.5 to about 5%, or about 3% to about 5%, of a hydrotrope. Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Bleaching Systems

The cleaning composition may contain 0-50% by weight, such as 1-40%, such as 1-30%, such as about 1% to about 20%, of a bleaching system. Any oxygen-based bleaching system comprising components known in the art for use in cleaning detergents may be utilized. Suitable bleaching system components include sources of hydrogen peroxide; peracids and sources of peracids (bleach activators); and bleach catalysts or boosters.

### Sources of Hydrogen Peroxide:

Suitable sources of hydrogen peroxide are inorganic persalts, including alkali metal salts such as sodium percarbonate and sodium perborates (usually mono- or tetrahydrate), and hydrogen peroxide-urea (1/1).

### Sources of Peracids:

Peracids may be (a) incorporated directly as preformed peracids or (b) formed in situ in the wash liquor from hydrogen peroxide and a bleach activator (perhydrolysis) or (c) formed in situ in the wash liquor from hydrogen peroxide and a perhydrolase and a suitable substrate for the latter, e.g., an ester.
a) Suitable preformed peracids include, but are not limited to, peroxycarboxylic acids such as peroxybenzoic acid and its ring-substituted derivatives, peroxy-α-naphthoic acid, peroxyphthalic acid, peroxylauric acid, peroxystearic acid, ε-phthalimidoperoxycaproic acid [phthalimidoperoxyhexanoic acid (PAP)], and o-carboxybenzamidoperoxycaproic acid; aliphatic and aromatic diperoxydicarboxylic acids such as diperoxydodecanedioic acid, diperoxyazelaic acid, diperoxysebacic acid, diperoxybrassylic acid, 2-decyldiperoxybutanedioic acid, and diperoxyphthalic, -isophthalic and -terephthalic acids; perimidic acids; peroxymonosulfuric acid; peroxydisulfuric acid; peroxyphosphoric acid; peroxysilicic acid; and mixtures of said compounds. It is understood that the peracids mentioned may in some cases be best added as suitable salts, such as alkali metal salts (e.g., Oxone®) or alkaline earth-metal salts.
b) Suitable bleach activators include those belonging to the class of esters, amides, imides, nitriles or anhydrides and, where applicable, salts thereof. Suitable examples are tetraacetylethylenediamine (TAED), sodium 4-[(3,5,5-trimethylhexanoyl)oxy]benzene-1-sulfonate (ISONOBS), sodium 4-(dodecanoyloxy)benzene-1-sulfonate (LOBS), sodium 4-(decanoyloxy)benzene-1-sulfonate, 4-(decanoyloxy)benzoic acid (DOBA), sodium 4-(nonanoyloxy)benzene-1-sulfonate (NOBS), and/or those disclosed in WO98/17767. A particular family of bleach activators of interest was disclosed in EP624154 and particularly preferred in that family is acetyl triethyl citrate (ATC). ATC or a short chain triglyceride like triacetin has the advantage that they are environmentally friendly. Furthermore, acetyl triethyl citrate and triacetin have good hydrolytical stability in the product upon storage and are efficient bleach activators. Finally, ATC is multifunctional, as the citrate released in the perhydrolysis reaction may function as a builder.

### Bleach Catalysts and Boosters

The bleaching system may also include a bleach catalyst or booster.

Some non-limiting examples of bleach catalysts that may be used in the compositions of the present invention include manganese oxalate, manganese acetate, manganese-collagen, cobalt-amine catalysts and manganese triazacyclononane (MnTACN) catalysts; particularly preferred are complexes of manganese with 1,4,7-trimethyl-1,4,7-triazacyclononane (Me3-TACN) or 1,2,4,7-tetramethyl-1,4,7-triazacyclononane (Me4-TACN), in particular Me3-TACN, such as the dinuclear manganese complex [(Me3-TACN)Mn(O)3Mn(Me3-TACN)](PF6)2, and [2,2′,2″-nitrilotris(ethane-1,2-diylazanylylidene-κN-methanylylidene)triphenolato-κ3O]manganese(III). The bleach catalysts may also be other metal compounds; such as iron or cobalt complexes.

In some embodiments, where a source of a peracid is included, an organic bleach catalyst or bleach booster may be used having one of the following formulae:

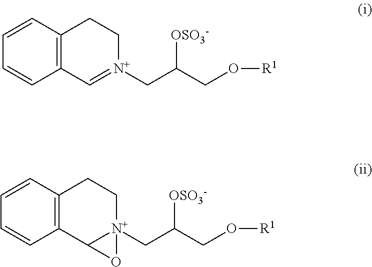

(iii) and mixtures thereof;
wherein each R1 is independently a branched alkyl group containing from 9 to 24 carbons or linear alkyl group containing from 11 to 24 carbons, preferably each R1 is independently a branched alkyl group containing from 9 to 18 carbons or linear alkyl group containing from 11 to 18 carbons, more preferably each R1 is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl.

Other exemplary bleaching systems are described, e.g. in WO2007/087258, WO2007/087244, WO2007/087259, EP1867708 (Vitamin K) and WO2007/087242.

Suitable photobleaches may for example be sulfonated zinc or aluminium phthalocyanines.

### Builders and Co-Builders

The detergent composition may contain about 0-65% by weight, such as about 5% to about 50%, 20-60% of a detergent builder or co-builder, or a mixture thereof. In a dishwashing detergent, the level of builder is typically in the range 40-65%, particularly in the range 50-65%. The builder and/or co-builder may particularly be a chelating agent that forms water-soluble complexes with Ca and Mg. Any builder and/or co-builder known in the art for use in cleaning detergents may be utilized.

Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Clariant), ethanolamines such as 2-aminoethan-1-ol (MEA), diethanolamine (DEA, also known as 2,2′-iminodiethan-1-ol), triethanolamine (TEA, also known as 2,2′,2″-nitrilotriethan-1-ol), and (carboxymethyl)inulin (CMI), and combinations thereof.

The detergent composition may also contain from about 0-50% by weight, such as about 5% to about 30%, of a detergent co-builder. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N, N′-disuccinic acid (EDDS), methylglycinediacetic acid (MGDA), glutamic acid-N,N-diacetic acid (GLDA), 1-hydroxyethane-1,1-diylbis(phosphonic acid (HEDP), ethylenediaminetetramethylenetetrakis (phosphonic acid) (EDTMPA), diethylenetriaminepentamethylenepentakis (phosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)aspartic acid (SMAS), N-(2-sulfoethyl)aspartic acid (SEAS), N-(2-sulfomethyl)glutamic acid (SMGL), N-(2-sulfoethyl)glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA), N-(2-hydroxyethyl)ethylenediamine-N,N′,N″-triacetic acid (HEDTA), diethanolglycine (DEG), aminotrimethylenetris(phosphonic acid) (ATMP), and combinations and salts thereof. Further exemplary builders and/or co-builders are described in, e.g., WO 09/102854, U.S. Pat. No. 5,977,053.

### Polymers

The detergent composition may contain 0.005-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties. Exemplary polymers include (carboxymethyl)cellulose (CMC), poly(vinyl alcohol) (PVA), poly(ethyleneglycol) or poly(ethylene oxide) (PEG or PEO), ethoxylated poly(ethyleneimine), (carboxymethyl)inulin (CMI), carboxylate polymers and d, and lauryl methacrylate/acrylic acid copolymers, hydrophobically modified CMC (HM-CMC), silicones, copolycarboxylates such as polyacrylates, maleic/acrylic acid copolymers, acrylate/styrene copolymers, poly(aspartic) acipolymers of terephthalic acid and oligomeric glycols, copolymers of poly(ethylene terephthalate) and poly(oxyethene terephthalate) (PET-POET), poly(vinylpyrrolidone) (PVP), poly(vinylimidazole) (PVI), poly(vinylpyridine-N-oxide) (PVPO or PVPNO) and copoly(vinylimidazole/vinylpyrrolidone) (PVPVI). Suitable examples include PVP-K15, PVP-K30, ChromaBond S-400, ChromaBond S-403E and Chromabond S-100 from Ashland Aqualon, and Sokalan® HP 165, Sokalan® HP 50 (Dispersing agent), Sokalan® HP 53 (Dispersing agent), Sokalan® HP 59 (Dispersing agent), Sokalan® HP 56 (dye transfer inhibitor), Sokalan® HP 66 K (dye transfer inhibitor) from BASF. Further exemplary polymers include sulfonated polycarboxylates, polyethylene oxide and polypropylene oxide (PEO-PPO) and diquaternium ethoxy sulfate. Particularly preferred polymer is ethoxylated homopolymer Sokalan® HP 20 from BASF, which helps to prevent redeposition of soil in the wash liquor. Further exemplary polymers include sulfonated polycarboxylates, ethylene oxide-propylene oxide copolymers (PEO-PPO), copolymers of PEG with and vinyl acetate, and diquaternium ethoxy sulfate or quaternized sulfated ethoxylated hexamethylenediamine. Other exemplary polymers are disclosed in, e.g., WO 2006/130575. Salts of the above-mentioned polymers are also contemplated.

### Adjunct Materials

Any detergent adjunct components known in the art may also be utilized. Exemplary adjunct materials may include anti-corrosion agents, anti-shrink agents, anti-soil redeposition agents, anti-wrinkling agents, bactericides, binders, corrosion inhibitors, disintegrates/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. The choice of such ingredients is well within the skill of the artisan.

### Dispersants

The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

### Dye Transfer Inhibiting Agents

The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

### Fluorescent Whitening Agent

The detergent compositions of the present invention will preferably also contain additional components that may tint articles being cleaned, such as fluorescent whitening agent or optical brighteners. Where present the brightener is preferably at a level of about 0.01% to about 0.5%. Any fluorescent whitening agent suitable for use in a cleaning detergent composition may be used in the composition of the present invention. The most commonly used fluorescent whitening agents are those belonging to the classes of diaminostilbene-sulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2.2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins.

Suitable fluorescent brightener levels include lower levels of from about 0.01, from 0.05, from about 0.1 or even from about 0.2 wt % to upper levels of 0.5 or even 0.75 wt %.

### Soil Release Polymers

The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalate based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore random graft co-polymers are suitable soil release polymers. Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

### Anti-Redeposition Agents

The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

### Formulation of Detergent Products

The detergent composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

Pouches can be configured as single or multicompartments. They can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid cleaning composition or part components and/or a liquid cleaning composition or part components separated by the water soluble film. The compartment for liquid components can be different in composition than compartments containing solids: US2009/0011970 A1.

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent.

A liquid or gel detergent may be non-aqueous.

### Laundry Soap Bars

The enzyme of the invention may be added to laundry soap bars and used for hand washing laundry, fabrics and/or textiles. The term laundry soap bar includes laundry bars, soap bars, combo bars, syndet bars and detergent bars. The types of bar usually differ in the type of surfactant they contain, and the term laundry soap bar includes those containing soaps from fatty acids and/or synthetic soaps. The laundry soap bar has a physical form which is solid and not a liquid, gel or a powder at room temperature. The term solid is defined as a physical form which does not significantly change over time, i.e. if a solid object (e.g. laundry soap bar) is placed inside a container, the solid object does not change to fill the container it is placed in. The bar is a solid typically in bar form but can be in other solid shapes such as round or oval.

The laundry soap bar may contain one or more additional enzymes, protease inhibitors such as peptide aldehydes (or hydrosulfite adduct or hemiacetal adduct), boric acid, borate, borax and/or phenylboronic acid derivatives such as 4-formylphenylboronic acid, one or more soaps or synthetic surfactants, polyols such as glycerine, pH controlling compounds such as fatty acids, citric acid, acetic acid and/or formic acid, and/or a salt of a monovalent cation and an organic anion wherein the monovalent cation may be for example Na⁺, K⁺ or NH₄⁺ and the organic anion may be for example formate, acetate, citrate or lactate such that the salt of a monovalent cation and an organic anion may be, for example, sodium formate.

The laundry soap bar may also contain complexing agents like EDTA and HEDP, perfumes and/or different type of fillers, surfactants e.g. anionic synthetic surfactants, builders, polymeric soil release agents, detergent chelators, stabilizing agents, fillers, dyes, colorants, dye transfer inhibitors, alkoxylated polycarbonates, suds suppressers, structurants, binders, leaching agents, bleaching activators, clay soil removal agents, anti-redeposition agents, polymeric dispersing agents, brighteners, fabric softeners, perfumes and/or other compounds known in the art.

The laundry soap bar may be processed in conventional laundry soap bar making equipment such as but not limited to: mixers, plodders, e.g a two stage vacuum plodder, extruders, cutters, logo-stampers, cooling tunnels and wrappers. The invention is not limited to preparing the laundry soap bars by any single method. The premix of the invention may be added to the soap at different stages of the process. For example, the premix containing a soap, a DNase of the present invention, optionally one or more additional enzymes, a protease inhibitor, and a salt of a monovalent cation and an organic anion may be prepared and the mixture is then plodded. The DNase of the present invention and optional additional enzymes may be added at the same time as the protease inhibitor for example in liquid form. Besides the mixing step and the plodding step, the process may further comprise the steps of milling, extruding, cutting, stamping, cooling and/or wrapping.

### Granular Detergent Compositions

Enzymes in the form of granules, comprising an enzyme-containing core and optionally one or more coatings, are commonly used in granular (powder) detergents. Various methods for preparing the core are well-known in the art and include, for example, a) spray drying of a liquid enzyme-containing solution, b) production of layered products with an enzyme coated as a layer around a pre-formed inert core particle, e.g. using a fluid bed apparatus, c) absorbing an enzyme onto and/or into the surface of a pre-formed core, d) extrusion of an enzyme-containing paste, e) suspending an enzyme-containing powder in molten wax and atomization to result in prilled products, f) mixer granulation by adding an enzyme-containing liquid to a dry powder composition of granulation components, g) size reduction of enzyme-containing cores by milling or crushing of larger particles, pellets, etc., and h) fluid bed granulation. The enzyme-containing cores may be dried, e.g. using a fluid bed drier or other known methods for drying granules in the feed or enzyme industry, to result in a water content of typically 0.1-10% w/w water.

The enzyme-containing cores are optionally provided with a coating to improve storage stability and/or to reduce dust formation. One type of coating that is often used for enzyme granulates for detergents is a salt coating, typically an inorganic salt coating, which may e.g. be applied as a solution of the salt using a fluid bed. Other coating materials that may be used are, for example, polyethylene glycol (PEG), methyl hydroxy-propyl cellulose (MHPC) and polyvinyl alcohol (PVA). The granules may contain more than one coating, for example a salt coating followed by an additional coating of a material such as PEG, MHPC or PVA.

For further information on enzyme granules and production thereof, see WO 2013/007594 as well as e.g. WO 2009/092699, EP 1705241, EP 1382668, WO 2007/001262, U.S. Pat. No. 6,472,364, WO 2004/074419 and WO 2009/102854.

### Formulation of Enzyme in Co-Granule

The enzyme of the invention may be formulated as a granule for example as a co-granule that combines one or more enzymes. Each enzyme will then be present in more granules securing a more uniform distribution of enzymes in the detergent. This also reduces the physical segregation of different enzymes due to different particle sizes. Methods for producing multi-enzyme co-granulates for the detergent industry are disclosed in the IP.com disclosure IPCOM000200739D.

Another example of formulation of enzymes by the use of co-granulates are disclosed in WO 2013/188331, which relates to a detergent composition comprising (a) a multi-enzyme co-granule; (b) less than 10 wt zeolite (anhydrous basis); and (c) less than 10 wt phosphate salt (anhydrous basis), wherein said enzyme co-granule comprises from 10 to 98 wt % moisture sink component and the composition additionally comprises from 20 to 80 wt % detergent moisture sink component. WO 2013/188331 also relates to a method of treating and/or cleaning a surface, preferably a fabric surface comprising the steps of (i) contacting said surface with the detergent composition as claimed and described herein in an aqueous wash liquor, (ii) rinsing and/or drying the surface.

The multi-enzyme co-granule may comprise an enzyme of the invention and (a) one or more enzymes selected from the group consisting of first-wash lipases, cleaning cellulases, xyloglucanases, perhydrolases, peroxidases, lipoxygenases, laccases and mixtures thereof; and (b) one or more enzymes selected from the group consisting of hemicellulases, proteases, care cellulases, cellobiose dehydrogenases, xylanases, phospho lipases, esterases, cutinases, pectinases, mannanases, pectate lyases, keratinases, reductases, oxidases, phenoloxidases, ligninases, pullulanases, tannases, pentosanases, lichenases, glucanases, arabinosidases, hyaluronidase, chondroitinase, amylases, and mixtures thereof.

The present invention is further described in the following non-limiting paragraphs:
1. A DNase variant, comprising at least two alterations at positions selected from the group consisting of positions 20, 26, 30, 32, 36, 37, 43, 46, 55, 68, 69, 76, 79, 82, 84, 86, 91, 92, 95, 97, 101, 102, 103, 105, 106, 107, 112, 125, 129, 133, 134, 138, 140, 141, 144, 146, 147, 148, 149, 150, 153, 154, 155, 157, 158, 159, 160, 170, 172, 185, 187, 191, 192, 197, 199, 208, 211, 213, 214, 217 and 218 of SEQ ID NO: 1, wherein the variant has a sequence identity of at least 80%, but less than 100%, to the polypeptide of SEQ ID NO: 1, wherein the variant has DNase activity, and preferably wherein the variant has improved stability and/or activity as compared to SEQ ID NO: 1, SEQ ID NO: 2 or SEQ ID NO: 3.
2. The DNase variant of paragraph 1, wherein the variant comprises at least two alterations selected from the group consisting of G20C, S26*, K30C, D32Q, K36C,H, G37C,R, F43W, D46G, A55I, N68D, S69V, A76I, K82S, T, P84D, T, K86G,L,N,Q,T,V,Y, A91R, L92E, K95I, P97E,N, A101E,N, Q102E, D103S, K105N,G,Q,T,D, S106C,D, F112Y,W, A125C, L129K, N133Q,R, S134C, V138C, N140H, G141Q,R, S144E, N146A, K147N,E,R, V148I, A149D,E,F, Q150D, P153D,V, S154E, K155E,F,L,S,T, Q157D,E, Q158D, T159Q, K160D, T170Q, A172D,E,H,R, K185*, V187N,Y, N191*, K192A, D197K,S, G199Q, Q208V, E211Y,T,P, N213S, N214D, N217A and Y218D,E, wherein positions correspond to the positions of SEQ ID NO: 1.
3. The DNase variant of paragraph 1 or 2, wherein the variant comprises at least two alterations selected from the group consisting of S26*, G37R, S69V, A76I, K86G,L,N,Q,T,V,Y, K95I, A101E, Q102E, K105N,G,Q,T,D, F112Y,W, L129K, N140H, G141Q,R, S144E, N146A, K147N,E, V148I, A149D,E,F, Q150D, P153D,V, S154E, K155E,F,L,S,T, Q157E, Q158D, T159Q, K160D, A172D,E,H,R, V187N,Y, K192A, D197K,S, G199Q, Q208V, E211Y,T,P, N214D, N217A and Y218D,E, wherein positions correspond to the positions of SEQ ID NO: 1.
4. The DNase variant of any one of paragraphs 1-3, wherein the variant comprises at least two alterations selected from the group consisting of S26*, G37R, S69V, A76I, K86G,L,N,Q,T,V,Y, K95I, A101E, Q102E, K105N,G,Q,T,D, L129K, G141Q,R, V148I, P153D,V, K155E,F,L,S,T, Q157E, T159Q, A172E, V187N,Y, D197K,S, N214D and Y218D,E, wherein positions correspond to the positions of SEQ ID NO: 1.
5. The DNase variant of any one of paragraphs 1-4, wherein the variant has a sequence identity of at least 85%, at least 90%, at least 95%, at least 96%, at least 97%, at least 98% or at least 99% to the polypeptide of SEQ ID NO: 1, SEQ ID NO: 2 or SEQ ID NO: 3.
6. The DNase variant of any one of paragraphs 1-5, wherein the total number of alterations of the DNase variant is 2-20, e.g. 3-15 or 4-12, such as 3, 4, 5, 6, 7, 8, 9, 10 or 11 alterations, compared to SEQ ID NO: 1, SEQ ID NO: 2 or SEQ ID NO: 3.
7. The DNase variant of any one of paragraphs 1-6, wherein the variant comprises at least one of the following sets of alterations:

| | |
|---|---|
| 1 | N133Q, V148I, L180S |
| 2 | D46G, N133Q, K185* |
| 3 | K86L, K155L, A172H |
| 4 | K86T, K155S, E211T |
| 5 | K86T, K155E, A172R |
| 6 | K86L, T170Q, V187Y |
| 7 | K36H, K86T, K155E |
| 8 | A91R, A149E, K155T |
| 9 | K86Y, Q102E, A149E |
| 10 | A40H, K155S, V187N |
| 11 | K105Q, V187N, Y209H |
| 12 | A55I, K86V, K155T |
| 13 | S144E, A149E, K155E, K192A |
| 14 | K105D, N214D, Y218E |
| 15 | K147E, A172E, V187N, K192A |
| 16 | S69V, Q102E, T159Q |
| 17 | K36C, V138C |
| 18 | L92E, K147N, Q150D, Q157D |
| 19 | S69V, K86T, L92E, V187N |
| 20 | K82S, K155F, N214D |
| 21 | N133Q, N146A, P153D, D197S |
| 22 | S69V, G79C, P107C |
| 23 | K105G, F112Y, A172E |
| 24 | K86Q, N140H, K147E, K155F |
| 25 | F112W, N214D, Y218D |
| 26 | K105N, N140H, K147E, S154E |
| 27 | Q102E, N214D, Y218D |
| 28 | D32Q, K86V, K155E |
| 29 | F112W, N214D, Y218E |
| 30 | K86L, K95I, A149E |
| 31 | K86L, A101E, E211Y |
| 32 | A101E, N214D, Y218D |
| 33 | A101E, N214D, Y218E |
| 34 | A149E, N214D, Y218D |
| 35 | A149E, N214D, Y218E |
| 36 | A149E, P153D, A172E, Q208V |
| 37 | D46G, A101E, G141Q |
| 38 | F43W, K155F, N217A |
| 39 | K105G, N214D, Y218E |
| 40 | K105N, A149D, N213S |
| 41 | K105N, A172R, V187N |
| 42 | K105N, K155F, E211P |
| 43 | K105N, K155L, D197K |
| 44 | K105Q, K155F, N191* |
| 45 | K105Q, K155T, E211T |
| 46 | K105Q, V187N, K192A, D197S |
| 47 | K105T, K155S, V187N |
| 48 | K147N, A172D, V187N, K192A |
| 49 | K147N, Q150D, S154E, D197S |
| 50 | K155E, D197S, N217A |
| 51 | K36C, K105G, V138C |
| 52 | K82T, N140H, K155E |
| 53 | K86G, A101E, V187Y |
| 54 | K86G, K105Q, K155S |
| 55 | K86N, N146A, Q150D, S154E |
| 56 | K86Q, A101E, K105Q, N146A |
| 57 | K86Q, A149E, K155E, A172D |
| 58 | K86T, A101E, G199Q |
| 59 | K86V, A149F, K155F, A172E |
| 60 | K86V, K95I, K155E |
| 61 | K86V, K95I, K155L |
| 62 | K95I, A172D, V187N, K192A |
| 63 | K95I, S144E, A149F, S154E |
| 64 | N68D, N140H, K147N, K155E |
| 65 | P84D, K155L, K160D, A172D |
| 66 | P84D, N140H, K147E, Q158D |
| 67 | P84T, A101E, F112Y |
| 68 | P97E, N140H, A149E, K155L |
| 69 | Q102E, N146A, S154E, K160D |
| 70 | Q102E, N214D, Y218E |
| 71 | S144E, A149F, K155L, D197S |
| 72 | S26*, K105N, G141Q |
| 73 | S69V, K86Q, L92E, K192A |
| 74 | V148I, P153D, A172E, K192A |
| 75 | A101E, A172E, N214D, Y218E |
| 76 | A101E, K105N, N214D, Y218E |
| 77 | S69V, Q102E, P153V, T159Q |
| 78 | S69V, Q102E, Q157E, T159Q |

8. An isolated polynucleotide encoding the DNase variant of any one of the preceding paragraphs.
9. A nucleic acid construct or expression vector capable of expressing the polynucleotide of paragraph 8.
10. A host cell comprising the polynucleotide of paragraph 8.
11. A method of producing a DNase variant, comprising:
   a) cultivating the host cell of paragraph 10 under conditions suitable for expression of the DNase variant; and
   b) recovering the DNase variant.
12. A detergent composition comprising a DNase variant of any one of paragraphs 1 to 7 and at least one detergent adjunct ingredient.
13. The composition of paragraph 12, wherein the composition comprises at least 0.002 ppm DNase variant, and:
   a) 2 wt % to 60 wt % of at least one surfactant, and/or
   b) 5 wt % to 50 wt % of at least one builder, and/or
   c) at least one additional enzyme.
14. The composition of paragraph 12 or 13, further comprising one or more additional enzymes selected from the group comprising of proteases, amylases, lipases, lichenases, cutinases, cellulases, endoglucanases, lichenases, xyloglucanases, pectinases, pectin lyases, xanthanases, peroxidases, haloperoxygenases, catalases and mannanases, or any mixture thereof.
15. The composition of any one of paragraphs 12-14, wherein the composition is in the form of a bar, a homogenous tablet, a tablet having two or more layers, a pouch having one or more compartments, a regular or compact powder, a granule, a paste, a gel, or a regular, compact or concentrated liquid.
16. Use of the DNase variants of any one of paragraphs 1-7 or the composition of any one of paragraphs 12-15 in a cleaning process, such as laundry or hard surface cleaning such as dishwashing.
17. A method for obtaining a DNase variant, comprising
   a) introducing into a DNase at least two alterations at positions selected from the group consisting of positions 26, 32, 36, 37, 43, 46, 55, 68, 69, 76, 79, 82, 84, 86, 91, 92, 95, 97, 101, 102, 105, 107, 112, 129, 133, 138, 140, 141, 144, 146, 147, 148, 149, 150, 153, 154, 155, 157, 158, 159, 160, 170, 172, 185, 187, 191, 192, 197, 199, 208, 211, 213, 214, 217 and 218 of SEQ ID NO: 1, wherein the variant has a sequence identity of at least 80%, but less than 100%, to the polypeptide of SEQ ID NO: 1, SEQ ID NO: 2 or SEQ ID NO: 3; and
   b) recovering the variant.
18. The method of paragraph 17, wherein the DNase variant comprises at least two alterations selected from the group consisting of S26*, D32Q, K36C,H, G37R, F43W, D46G, A55I, N68D, S69V, A76I, K82S,T, P84D, T, K86G,L,N,Q,T,V,Y, A91R, L92E, K95I, P97E,N, A101E, Q102E, K105N,G,Q,T,D, F112Y,W, L129K, N133Q, V138C, N140H, G141Q,R, S144E, N146A, K147N,E, V148I, A149D,E,F, Q150D, P153D,V, S154E, K155E,F,L,S,T, Q157D,E, Q158D, T159Q, K160D, T170Q, A172D,E,H,R, K185*, V187N,Y, N191*, K192A, D197K,S, G199Q, Q208V, E211Y,T,P, N213S, N214D, N217A and Y218D,E, wherein positions correspond to the positions of SEQ ID NO: 1.
19. The method of paragraph 17 or 18, wherein the DNase variant comprises at least two alterations selected from the group consisting of S26*, G37R, S69V, A76I, K86G,L,N,Q,T,V,Y, K95I, A101E, Q102E, K105N,G,Q,T,D, F112Y,W, L129K, N140H, G141Q,R, S144E, N146A, K147N,E, V148I, A149D,E,F, Q150D, P153D,V, S154E, K155E,F,L,S,T, Q157E, Q158D, T159Q, K160D, A172D,E,H,R, V187N,Y, K192A, D197K,S, G199Q, Q208V, E211Y,T,P, N214D, N217A and Y218D,E, wherein the positions correspond to positions of SEQ ID NO: 1.
20. The method of any one of paragraphs 17-19, wherein the variant comprises at least two alterations selected from the group consisting of S26*, G37R, S69V, A76I, K86G,L,N,Q,T,V,Y, K95I, A101E, Q102E, K105N,G,Q,T,D, L129K, G141Q,R, V148I, P153D,V, K155E,F,L,S,T, Q157E, T159Q, A172E, V187N,Y, D197K,S, N214D and Y218D, E, wherein positions correspond to the positions of SEQ ID NO: 1.
21. The method of any one of paragraphs 17-20, wherein the variant has a sequence identity of at least 85%, at least 90%, at least 95%, at least 96%, at least 97%, at least 98% or at least 99% to the polypeptide of SEQ ID NO: 1, SEQ ID NO: 2 or SEQ ID NO: 3.
22. The method of any one of paragraphs 17-21, wherein the total number of alterations of the DNase variant is 2-20, e.g. 3-15 or 4-12, such as 3, 4, 5, 6, 7, 8, 9, 10 or 11 alterations, compared to SEQ ID NO: 1.
23. The method of any one of paragraphs 17-22, wherein the variant comprises at least one of the following sets of alterations:

| | |
|---|---|
| 1 | N133Q, V148I, L180S |
| 2 | D46G, N133Q, K185* |
| 3 | K86L, K155L, A172H |
| 4 | K86T, K155S, E211T |
| 5 | K86T, K155E, A172R |
| 6 | K86L, T170Q, V187Y |
| 7 | K36H, K86T, K155E |
| 8 | A91R, A149E, K155T |
| 9 | K86Y, Q102E, A149E |
| 10 | A40H, K155S, V187N |
| 11 | K105Q, V187N, Y209H |
| 12 | A55I, K86V, K155T |
| 13 | S144E, A149E, K155E, K192A |
| 14 | K105D, N214D, Y218E |
| 15 | K147E, A172E, V187N, K192A |
| 16 | S69V, Q102E, T159Q |
| 17 | K36C, V138C |
| 18 | L92E, K147N, Q150D, Q157D |
| 19 | S69V, K86T, L92E, V187N |
| 20 | K82S, K155F, N214D |
| 21 | N133Q, N146A, P153D, D197S |
| 22 | S69V, G79C, P107C |
| 23 | K105G, F112Y, A172E |
| 24 | K86Q, N140H, K147E, K155F |
| 25 | F112W, N214D, Y218D |
| 26 | K105N, N140H, K147E, S154E |
| 27 | Q102E, N214D, Y218D |
| 28 | D32Q, K86V, K155E |
| 29 | F112W, N214D, Y218E |
| 30 | K86L, K95I, A149E |
| 31 | K86L, A101E, E211Y |
| 32 | A101E, N214D, Y218D |
| 33 | A101E, N214D, Y218E |
| 34 | A149E, N214D, Y218D |
| 35 | A149E, N214D, Y218E |
| 36 | A149E, P153D, A172E, Q208V |
| 37 | D46G, A101E, G141Q |
| 38 | F43W, K155F, N217A |
| 39 | K105G, N214D, Y218E |
| 40 | K105N, A149D, N213S |
| 41 | K105N, A172R, V187N |
| 42 | K105N, K155F, E211P |
| 43 | K105N, K155L, D197K |
| 44 | K105Q, K155F, N191* |
| 45 | K105Q, K155T, E211T |
| 46 | K105Q, V187N, K192A, D197S |
| 47 | K105T, K155S, V187N |
| 48 | K147N, A172D, V187N, K192A |
| 49 | K147N, Q150D, S154E, D197S |
| 50 | K155E, D197S, N217A |
| 51 | K36C, K105G, V138C |
| 52 | K82T, N140H, K155E |
| 53 | K86G, A101E, V187Y |
| 54 | K86G, K105Q, K155S |
| 55 | K86N, N146A, Q150D, S154E |
| 56 | K86Q, A101E, K105Q, N146A |
| 57 | K86Q, A149E, K155E, A172D |
| 58 | K86T, A101E, G199Q |
| 59 | K86V, A149F, K155F, A172E |
| 60 | K86V, K95I, K155E |
| 61 | K86V, K95I, K155L |
| 62 | K95I, A172D, V187N, K192A |
| 63 | K95I, S144E, A149F, S154E |
| 64 | N68D, N140H, K147N, K155E |
| 65 | P84D, K155L, K160D, A172D |
| 66 | P84D, N140H, K147E, Q158D |
| 67 | P84T, A101E, F112Y |
| 68 | P97E, N140H, A149E, K155L |
| 69 | Q102E, N146A, S154E, K160D |
| 70 | Q102E, N214D, Y218E |
| 71 | S144E, A149F, K155L, D197S |
| 72 | S26*, K105N, G141Q |
| 73 | S69V, K86Q, L92E, K192A |
| 74 | V148I, P153D, A172E, K192A |
| 75 | A101E, A172E, N214D, Y218E |
| 76 | A101E, K105N, N214D, Y218E |
| 77 | S69V, Q102E, P153V, T159Q |
| 78 | S69V, Q102E, Q157E, T159Q |

24. A method for cleaning an item, comprising exposing the item to a wash liquor comprising the DNase variant of any one of paragraphs 1-7 or the composition of any one of paragraphs 12-15, wherein the item is e.g. a textile or a hard surface.
25. The DNase variant of any one of paragraphs 1-7, wherein the variant has improved storage stability in a liquid detergent compared to a parent DNase without said alterations or to the DNase of SEQ ID NO: 1.
26. The DNase of paragraph 25, wherein the DNase has a residual activity (RA), determined e.g. as described in Example 2 herein, of at least 0.2, more preferably at least 0.3, such as at least 0.4, at least 0.5, at least 0.6, at least 0.7, at least 0.8 or at least 0.9, and most preferably 1.

### Examples

### Assays, Detergent Compositions and Media

### Model Detergent A2 (Liquid)

Ingredients: 12% LAS, 12% AEO, 4% AEOS/SLES, 2% MPG (monopropylene glycol), 3% ethanol, 2% TEA, 3% soap, 0.5% NaOH, 3.9% sodium citrate, 1.5% DTMPA Na7, 0.5% phenoxyethanol, and water to 100% (all percentages are w/w)

### MDU-2 Medium

Ingredients per liter medium: 45 g maltose, 1 g MgSO₄·7H₂O, 1 g NaCl, 2 g K₂SO₄, 12 g KH₂PO₄, 0.5 ml trace metal mixture, 0.1 ml DOWFAX™ 63N10 nonionic surfactant, 60 g maltose syrup (75%), and ion-exchanged water to 1000 ml. The trace metal mixture contains, per liter, 0.04 g di-sodium tetraborate, 0.4 g copper sulfate, 0.8 g iron sulfate, 0.7 g manganese sulfate, 0.8 g sodium molybdate, 8 g zinc sulfate and 2 g citric acid.

### Assay I: Testing of DNase Activity

DNase activity was determined on DNase Test Agar with Methyl Green (BD, Franklin Lakes, NJ, USA), which was prepared according to the supplier's manual. Briefly, 21 g of agar was dissolved in 500 ml water and then autoclaved for 15 min at 121° C. Autoclaved agar was temperated to 48° C. in water bath, and 20 ml of agar was poured into petri dishes and allowed to solidify by incubation overnight at room temperature. On solidified agar plates, 5 μl of enzyme solutions are added and DNase activity is observed as colorless zones around the spotted enzyme solutions.

### Assay II: Analysis of E-2-Nonenal on Textile Using an Electronic Nose.

One way of testing for the presence of malodor on textiles is by using E-2-Nonenal as a marker for the malodor, as this compound contributes to malodor on laundry. A solution of E-2-nonenal is added to a 5 cm×5 cm textile swatch and the swatch is placed in a 20 mL glass vial for GC analysis, and the vial is capped. 5 mL headspace from the capped vials is analyzed in a Heracles II Electronic nose from Alpha M.O.S., France (double column gas chromatograph with 2 FIDs, column 1: MXT5 and column 2: MXT1701) after 20 minutes incubation at 40° C.

### Methods

General methods of PCR, cloning, ligation nucleotides etc. are well-known to a person skilled in the art and may for example be found in in “Molecular cloning: A laboratory manual”, Sambrook et al. (1989), Cold Spring Harbor lab., Cold Spring Harbor, NY; Ausubel, F. M. et al. (eds.); “Current protocols in Molecular Biology”, John Wiley and Sons, (1995); Harwood, C. R., and Cutting, S. M. (eds.); “DNA Cloning: A Practical Approach, Volumes I and II”, D. N. Glover ed. (1985); “Oligonucleotide Synthesis”, M. J. Gait ed. (1984); “Nucleic Acid Hybridization”, B. D. Hames & S. J. Higgins eds (1985); “A Practical Guide To Molecular Cloning”, B. Perbal, (1984).

### Example 1: Preparation of DNase Variants

Site-directed variants were generated using specific primers that contained the desired new mutation. The new codon selected was the codon with the highest natural abundance for the specific amino acid in Aspergillus oryzae. The transformation substrate was made using PCR:

### 1) Separate Amplification of N-Terminal and C-Terminal Fragment, Relative to the Mutation Site.

The N-terminal fragment was amplified using a universal forward primer and a position specific reverse primer. The C-terminal fragment was amplified using a mutation specific forward primer and universal reverse primer. Both universal primers are complementary to sequences necessary for homologous integration in Aspergillus genome.

### 2) Assembly of the N-Terminal and C-Terminal Fragments In Vivo Recombination in the Cells.

The resulting transformation substrate was transformed into Aspergillus oryzae and transformants were selected for by growth on nitrate as the sole nitrogen source. Three single colonies of each type were picked into microtiter plates and grown for 4 days at 30° C. in broth specific for Aspergillus. The supernatants were used for screening.

### Example 2: Testing DNase Variants for Stability

The stability of DNase variants compared to DNase with SEQ ID NO: 1 (i.e., wild type) was evaluated by determining residual activity by incubating the DNase samples under conditions described below in a model detergent solution (Model detergent A2). A higher residual activity indicates a better or improved stability in detergent.

Spores from the Aspergillus transformations were incubated for 7 days in 220 μl MDU-2 media with 10 mM NaNO₃ in a 96 microwell plate (assay plate) at 30° C. Fermentation broth from the assay plate was assayed for residual activity using DNaseAlert™ substrate. Fermentation broth (20 μl) was added to 180 μl Model detergent A2 or buffer (50 mM TRIS pH 7.5, 5 mM MgCl₂, 0.01% Model detergent A2) and incubated for 30 min at room temperature. Following incubation, fermentation broth in buffer or detergent was diluted 10,000 times. The concentration of the DNaseAlert™ substrate in the reaction was 8 nM. Intensities (536 nm excitation, 556 nm emission) were measured continuously for 10 min, pH 7.5 at room temperature using a Tecan Infinite® M1000Pro microplate reader.

The measured intensities were plotted against time and the slopes were calculated from linear regression lines. Residual activity was calculated by taking the ratio of the measured slopes for the DNase variants incubated in detergent and slopes for the variants incubated in buffer. Residual activity (RA) values range from zero to one and refer to the activity that remains after exposure to Model detergent A2 for 30 min (RA₃₀ₘᵢₙ). The value 0 means that no remaining activity following Model detergent A2 treatment was measured, and the value 1 signifies 100% activity compared to the reference sample (buffer).

Measured residual activity values for DNase variants and the wild-type DNase (SEQ ID NO: 1) after incubation for 30 min (RA₃₀ₘᵢₙ) are summarized in Table 1 below. The table shows, for each individual substitution, the RA value for the substitution alone followed by the RA value for one or more variants having that substitution together with one or more additional substitutions.

TABLE 1
Residual activity (RA₃₀ₘᵢₙ) of DNase variants
and wildtype DNase

| | | Residual |
|---|---|---|
| Sample | | activity |
| # | Alterations | (RA) |
| 1 | K36H | 0.45 |
| 2 | K36H, K86T, K155E | 0.73 |
| 3 | K36C, K105G, V138C | 1.00 |
| 4 | K36C, V138C | 0.88 |
| 5 | N68D | 0.37 |
| 6 | N68D, N140H, K147N, K155E | 1.00 |
| 7 | S69V | 0.73 |
| 8 | S69V, K86Q, L92E, K192A | 1.00 |
| 9 | S69V, G79C, P107C | 0.91 |
| 10 | S69V, K86T, L92E, V187N | 0.88 |
| 11 | S69V, Q102E, T159Q | 0.87 |
| 12 | S69V Q102E Q157E T159Q | 1.00 |
| 13 | S69V Q102E P153V T159Q | 1.00 |
| 14 | K82S | 0.71 |
| 15 | K82S, K155F, N214D | 0.89 |
| 16 | P84D | 0.89 |
| 17 | P84D, K155L, K160D, A172D | 1.00 |
| 18 | P84D, N140H, K147E, Q158D | 1.00 |
| 19 | P84T, A101E, F112Y | 1.00 |
| 20 | K86G | 0.51 |
| 21 | K86G, K105Q, K155S | 1.00 |
| 22 | K86G, A101E, V187Y | 1.00 |
| 23 | K86L | 0.51 |
| 24 | K86L, A101E, E211Y | 0.99 |
| 25 | K86L, K951, A149E | 0.98 |
| 26 | K86L, T170Q, V187Y | 0.71 |
| 27 | K86L, K155L, A172H | 0.64 |
| 28 | K86N | 0.43 |
| 29 | K86N, N146A, Q150D, S154E | 1.00 |
| 30 | K86Q | 0.25 |
| 31 | K86Q, A101E, K105Q, N146A | 1.00 |
| 32 | S69V, K86Q, L92E, K192A | 1.00 |
| 33 | K86Q, A149E, K155E, A172D | 1.00 |
| 34 | K86Q, N140H, K147E, K155F | 0.92 |
| 35 | K86T | 0.54 |
| 36 | K86T, A101E, G199Q | 1.00 |
| 37 | S69V, K86T, L92E, V187N | 0.88 |
| 38 | K36H, K86T, K155E | 0.73 |
| 39 | K86T, K155E, A172R | 0.69 |
| 40 | K86T, K155S, E211T | 0.64 |
| 41 | K86V | 0.55 |
| 42 | K86V, K95I, K155E | 1.00 |
| 43 | K86V, A149F, K155F, A172E | 1.00 |
| 44 | K86V, K95I, K155L | 1.00 |
| 45 | D32Q, K86V, K155E | 0.97 |
| 46 | K86Y | 0.44 |
| 47 | K86Y, Q102E, A149E | 0.77 |
| 48 | K95I | 0.36 |
| 49 | K86V, K95I, K155E | 1.00 |
| 50 | K95I, A172D, V187N, K192A | 1.00 |
| 51 | K95I, S144E, A149F, S154E | 1.00 |
| 52 | K86V, K95I, K155L | 1.00 |
| 53 | K86L, K95I, A149E | 0.98 |
| 54 | P97E | 0.29 |
| 55 | P97E, N140H, A149E, K155L | 1.00 |
| 56 | A101E | 0.80 |
| 57 | K86Q, A101E, K105Q, N146A | 1.00 |
| 58 | A101E, N214D, Y218E | 1.00 |
| 59 | K86G, A101E, V187Y | 1.00 |
| 60 | P84T, A101E, F112Y | 1.00 |
| 61 | A101E, N214D, Y218D | 1.00 |
| 62 | A101E A172E N214D Y218E | 1.00 |
| 63 | A101E K105N N214D Y218E | 1.00 |
| 64 | K86T, A101E, G199Q | 1.00 |
| 65 | D46G, A101E, G141Q | 1.00 |
| 66 | K86L, A101E, E211Y | 0.99 |
| 67 | K105N | 0.72 |
| 68 | K105N, A149D, N213S | 1.00 |
| 69 | K105N, K155L, D197K | 1.00 |
| 70 | K105N, K155F, E211P | 1.00 |
| 71 | S26*, K105N, G141Q | 1.00 |
| 72 | K105N, A172R, V187N | 1.00 |
| 73 | K105N, N140H, K147E, S154E | 0.95 |
| 74 | K105Q | 0.47 |
| 75 | K86G, K105Q, K155S | 1.00 |
| 76 | K86Q, A101E, K105Q, N146A | 1.00 |
| 77 | K105Q, K155F, N191* | 1.00 |
| 78 | K105Q, V187N, K192A, D197S | 1.00 |
| 79 | K105Q, K155T, E211T | 1.00 |
| 80 | K105T | 0.43 |
| 81 | K105T, K155S, V187N | 1.00 |
| 82 | N133Q | 0.22 |
| 83 | N133Q, N146A, P153D, D197S | 0.91 |
| 84 | D46G, N133Q, K185* | 0.57 |
| 85 | S144E | 0.45 |
| 86 | S144E, A149F, K155L, D197S | 1.00 |
| 87 | K951, S144E, A149F, S154E | 1.00 |
| 88 | S144E, A149E, K155E, K192A | 0.84 |
| 89 | N146A | 0.32 |
| 90 | K86Q, A101E, K105Q, N146A | 1.00 |
| 91 | K86N, N146A, Q150D, S154E | 1.00 |
| 92 | Q102E, N146A, S154E, K160D | 1.00 |
| 93 | N133Q, N146A, P153D, D197S | 0.91 |
| 94 | K147N | 0.46 |
| 95 | N68D, N140H, K147N, K155E | 1.00 |
| 96 | K147N, Q150D, S154E, D197S | 1.00 |
| 97 | K147N, A172D, V187N, K192A | 1.00 |
| 98 | L92E, K147N, Q150D, Q157D | 0.88 |
| 99 | V148I | 0.30 |
| 100 | V148I, P153D, A172E, K192A | 1.00 |
| 101 | A149F | 0.84 |
| 102 | K86V, A149F, K155F, A172E | 1.00 |
| 103 | S144E, A149F, K155L, D197S | 1.00 |
| 104 | K95I, S144E, A149F, S154E | 1.00 |
| 105 | S154E | 0.68 |
| 106 | K147N, Q150D, S154E, D197S | 1.00 |
| 107 | K86N, N146A, Q150D, S154E | 1.00 |
| 108 | Q102E, N146A, S154E, K160D | 1.00 |
| 109 | K95I, S144E, A149F, S154E | 1.00 |
| 110 | K105N, N140H, K147E, S154E | 0.95 |
| 111 | K155E | 0.76 |
| 112 | K86V, K95I, K155E | 1.00 |
| 113 | K155E, D197S, N217A | 1.00 |
| 114 | K82T, N140H, K155E | 1.00 |
| 115 | K86Q, A149E, K155E, A172D | 1.00 |
| 116 | N68D, N140H, K147N, K155E | 1.00 |
| 117 | D32Q, K86V, K155E | 0.97 |
| 118 | S144E, A149E, K155E, K192A | 0.84 |
| 119 | K155F | 0.39 |
| 120 | K86V, A149F, K155F, A172E | 1.00 |
| 121 | F43W, K155F, N217A | 1.00 |
| 122 | K105Q, K155F, N191* | 1.00 |
| 123 | K105N, K155F, E211P | 1.00 |
| 124 | K86Q, N140H, K147E, K155F | 0.92 |
| 125 | K82S, K155F, N214D | 0.89 |
| 126 | K155L | 0.56 |
| 127 | P84D, K155L, K160D, A172D | 1.00 |
| 128 | K105N, K155L, D197K | 1.00 |
| 129 | S144E, A149F, K155L, D197S | 1.00 |
| 130 | K86V, K95I, K155L | 1.00 |
| 131 | P97E, N140H, A149E, K155L | 1.00 |
| 132 | K86L, K155L, A172H | 0.64 |
| 133 | K155S | 0.51 |
| 134 | K86G, K105Q, K155S | 1.00 |
| 135 | K105T, K155S, V187N | 1.00 |
| 136 | A40H, K155S, V187N | 0.79 |
| 137 | K86T, K155S, E211T | 0.64 |
| 138 | K155T | 0.55 |
| 139 | K105Q, K155T, E211T | 1.00 |
| 140 | A55I, K86V, K155T | 0.83 |
| 141 | A91R, A149E, K155T | 0.77 |
| 142 | Q157D | 0.24 |
| 143 | L92E, K147N, Q150D, Q157D | 0.88 |
| 144 | Q157E | 0.29 |
| 145 | S69V Q102E Q157E T159Q | 1.00 |
| 146 | Q158D | 0.32 |
| 147 | P84D, N140H, K147E, Q158D | 1.00 |
| 148 | K160D | 0.40 |
| 149 | P84D, K155L, K160D, A172D | 1.00 |
| 150 | Q102E, N146A, S154E, K160D | 1.00 |
| 151 | A172D | 0.45 |
| 152 | P84D, K155L, K160D, A172D | 1.00 |
| 153 | K86Q, A149E, K155E, A172D | 1.00 |
| 154 | K95I, A172D, V187N, K192A | 1.00 |
| 155 | K147N, A172D, V187N, K192A | 1.00 |
| 156 | A172E | 0.41 |
| 157 | K86V, A149F, K155F, A172E | 1.00 |
| 158 | V148I, P153D, A172E, K192A | 1.00 |
| 159 | A149E, P153D, A172E, Q208V | 1.00 |
| 160 | K105G, F112Y, A172E | 0.92 |
| 161 | K147E, A172E, V187N, K192A | 0.86 |
| 162 | K951, A172D, V187N, K192A | 1.00 |
| 163 | K105Q, V187N, K192A, D197S | 1.00 |
| 164 | K147N, A172D, V187N, K192A | 1.00 |
| 165 | K105N, A172R, V187N | 1.00 |
| 166 | K105T, K155S, V187N | 1.00 |
| 167 | S69V, K86T, L92E, V187N | 0.88 |
| 168 | K147E, A172E, V187N, K192A | 0.86 |
| 169 | K105Q, V187N, Y209H | 0.82 |
| 170 | A40H, K155S, V187N | 0.79 |
| 171 | K86G, A101E, V187Y | 1.00 |
| 172 | K86L, T170Q, V187Y | 0.71 |
| 173 | K192A | 0.76 |
| 174 | S69V, K86Q, L92E, K192A | 1.00 |
| 175 | V148I, P153D, A172E, K192A | 1.00 |
| 176 | K95I, A172D, V187N, K192A | 1.00 |
| 177 | K105Q, V187N, K192A, D197S | 1.00 |
| 178 | K147N, A172D, V187N, K192A | 1.00 |
| 179 | K147E, A172E, V187N, K192A | 0.86 |
| 180 | S144E, A149E, K155E, K192A | 0.84 |
| 181 | D197S | 0.51 |
| 182 | K155E, D197S, N217A | 1.00 |
| 183 | S144E, A149F, K155L, D197S | 1.00 |
| 184 | K147N, Q150D, S154E, D197S | 1.00 |
| 185 | K105Q, V187N, K192A, D197S | 1.00 |
| 186 | N133Q, N146A, P153D, D197S | 0.91 |
| 187 | Q208V | 0.83 |
| 188 | A149E, P153D, A172E, Q208V | 1.00 |
| 189 | N214D | 0.35 |
| 190 | A149E, N214D, Y218E | 1.00 |
| 191 | A101E, N214D, Y218E | 1.00 |
| 192 | A149E, N214D, Y218D | 1.00 |
| 193 | K105G, N214D, Y218E | 1.00 |
| 194 | A101E, N214D, Y218D | 1.00 |
| 195 | Q102E, N214D, Y218E | 1.00 |
| 196 | F112W, N214D, Y218E | 0.97 |
| 197 | Q102E, N214D, Y218D | 0.96 |
| 198 | F112W, N214D, Y218D | 0.94 |
| 199 | K82S, K155F, N214D | 0.89 |
| 200 | K105D, N214D, Y218E | 0.86 |
| 201 | N214D, Y218E | 0.37 |
| 202 | N214D, Y218D | 0.36 |
| 203 | N217A | 0.84 |
| 204 | K155E, D197S, N217A | 1.00 |
| 205 | F43W, K155F, N217A | 1.00 |
| 206 | Wild-type (SEQ ID NO: 1) | 0.16 |

### Example 3: DNase Variant Thermostability

The thermal stability of non-purified DNase variants of SEQ ID NO: 1 was estimated by differential scanning fluorimetry (nanoDSF, Prometheus NT.48 or NT.Plex, NanoTemper Technologies GmbH, Germany), which measures the change in intrinsic tryptophan and tyrosine fluorescence due to unfolding upon temperature increase (20° C.-95° C., rate of increase 3.3° C./min). Melting temperatures are calculated from the inflection points in the melting curve using the first and second derivative. Expressed variants (7 days in MDU-2, 10 mM NaNO₃, 30° C.) in supernatants were diluted 1:1 in a 500 mM HEPES pH 8, 10 mM MgCl₂. The results are expressed below in Table 2 as a melting temperature, Tm.

TABLE 2
Melting temperature (Tm) of DNase variants and
wild-type DNase

| Substitutions compared to SEQ ID NO: 1 | Tm (° C.) |
|---|---|
| Wild-type (SEQ ID NO: 1) | 77.7 |
| S69V, K105D, K147E | 79.6 |
| S69V, A149E, D197K | 77.9 |
| S106D, N133R | 77.9 |
| S106C, A125C | 79.6 |
| Q102E, K105D, A149E, N213S | 80.3 |
| K105N, K147R, V187N | 79.6 |
| G37C, S134C | 78.4 |
| G20C, K30C | 85.0 |
| A101N, D103S | 78.2 |

1. A DNase variant, comprising at least two alterations at positions selected from the group consisting of positions 69, 102, 26, 32, 36, 37, 43, 46, 55, 68, 76, 79, 82, 84, 86, 91, 92, 95, 97, 101, 105, 107, 112, 129, 133, 138, 140, 141, 144, 146, 147, 148, 149, 150, 153, 154, 155, 157, 158, 159, 160, 170, 172, 185, 187, 191, 192, 197, 199, 208, 211, 213, 214, 217 and 218 of SEQ ID NO: 1, wherein the variant has a sequence identity of at least 80%, but less than 100%, to the polypeptide of SEQ ID NO: 1, SEQ ID NO: 2 or SEQ ID NO: 3; wherein the variant has DNase activity and wherein the variant has improved stability and/or activity as compared to SEQ ID NO: 1.

2. The DNase variant of claim 1, wherein the variant comprises at least two alterations selected from the group consisting of S69V, Q102E, S26*, D32Q, K36C,H, G37R, F43W, D46G, A55I, N68D, A76I, K82S, T, P84D, T, K86G,L,N,Q,T,V,Y, A91R, L92E, K95I, P97E,N, A101E, K105N,G,Q,T,D, F112Y,W, L129K, N133Q, V138C, N140H, G141Q,R, S144E, N146A, K147N,E, V148I, A149D,E,F, Q150D, P153D,V, S154E, K155E,F,L,S,T, Q157D,E, Q158D, T159Q, K160D, T170Q, A172D,E,H,R, K185*, V187N,Y, N191*, K192A, D197K,S, G199Q, Q208V, E211Y,T,P, N213S, N214D, N217A and Y218D,E, wherein positions correspond to the positions of SEQ ID NO: 1.

3. The DNase variant of claim 1, wherein the variant comprises at least two alterations selected from the group consisting of S69V, Q102E, S26*, G37R, A76I, K86G,L,N,Q,T,V,Y, K95I, A101E, K105N,G,Q,T,D, F112Y,W, L129K, N140H, G141Q,R, S144E, N146A, K147N,E, V148I, A149D,E,F, Q150D, P153D,V, S154E, K155E,F,L,S,T, Q157E, Q158D, T159Q, K160D, A172D,E,H,R, V187N,Y, K192A, D197K,S, G199Q, Q208V, E211Y,T,P, N214D, N217A and Y218D,E, wherein positions correspond to positions of SEQ ID NO: 1.

4. The DNase variant of claim 1, wherein the variant comprises at least two alterations selected from the group consisting of S69V, Q102E, S26*, G37R, A76I, K86G,L,N,Q,T,V,Y, K95I, A101E, K105N,G,Q,T,D, L129K, G141Q,R, V148I, P153D,V, K155E,F,L,S,T, Q157E, T159Q, A172E, V187N,Y, D197K,S, N214D and Y218D,E, wherein positions correspond to the positions of SEQ ID NO: 1.

5. The DNase variant of claim 1, wherein the variant has a sequence identity of at least 85% to the polypeptide of SEQ ID NO: 1, SEQ ID NO: 2 or SEQ ID NO: 3.

6. The DNase variant of claim 1, wherein the variant comprises at least one of the following sets of alterations:

| | |
|---|---|
| 1 | N133Q, V148I, L180S |
| 2 | D46G, N133Q, K185* |
| 3 | K86L, K155L, A172H |
| 4 | K86T, K155S, E211T |
| 5 | K86T, K155E, A172R |
| 6 | K86L, T170Q, V187Y |
| 7 | K36H, K86T, K155E |
| 8 | A91R, A149E, K155T |
| 9 | K86Y, Q102E, A149E |
| 10 | A40H, K155S, V187N |
| 11 | K105Q, V187N, Y209H |
| 12 | A55I, K86V, K155T |
| 13 | S144E, A149E, K155E, K192A |
| 14 | K105D, N214D, Y218E |
| 15 | K147E, A172E, V187N, K192A |
| 16 | S69V, Q102E, T159Q |
| 17 | K36C, V138C |
| 18 | L92E, K147N, Q150D, Q157D |
| 19 | S69V, K86T, L92E, V187N |
| 20 | K82S, K155F, N214D |
| 21 | N133Q, N146A, P153D, D197S |
| 22 | S69V, G79C, P107C |
| 23 | K105G, F112Y, A172E |
| 24 | K86Q, N140H, K147E, K155F |
| 25 | F112W, N214D, Y218D |
| 26 | K105N, N140H, K147E, S154E |
| 27 | Q102E, N214D, Y218D |
| 28 | D32Q, K86V, K155E |
| 29 | F112W, N214D, Y218E |
| 30 | K86L, K95I, A149E |
| 31 | K86L, A101E, E211Y |
| 32 | A101E, N214D, Y218D |
| 33 | A101E, N214D, Y218E |
| 34 | A149E, N214D, Y218D |
| 35 | A149E, N214D, Y218E |
| 36 | A149E, P153D, A172E, Q208V |
| 37 | D46G, A101E, G141Q |
| 38 | F43W, K155F, N217A |
| 39 | K105G, N214D, Y218E |
| 40 | K105N, A149D, N213S |
| 41 | K105N, A172R, V187N |
| 42 | K105N, K155F, E211P |
| 43 | K105N, K155L, D197K |
| 44 | K105Q, K155F, N191* |
| 45 | K105Q, K155T, E211T |
| 46 | K105Q, V187N, K192A, D197S |
| 47 | K105T, K155S, V187N |
| 48 | K147N, A172D, V187N, K192A |
| 49 | K147N, Q150D, S154E, D197S |
| 50 | K155E, D197S, N217A |
| 51 | K36C, K105G, V138C |
| 52 | K82T, N140H, K155E |
| 53 | K86G, A101E, V187Y |
| 54 | K86G, K105Q, K155S |
| 55 | K86N, N146A, Q150D, S154E |
| 56 | K86Q, A101E, K105Q, N146A |
| 57 | K86Q, A149E, K155E, A172D |
| 58 | K86T, A101E, G199Q |
| 59 | K86V, A149F, K155F, A172E |
| 60 | K86V, K95I, K155E |
| 61 | K86V, K95I, K155L |
| 62 | K95I, A172D, V187N, K192A |
| 63 | K95I, S144E, A149F, S154E |
| 64 | N68D, N140H, K147N, K155E |
| 65 | P84D, K155L, K160D, A172D |
| 66 | P84D, N140H, K147E, Q158D |
| 67 | P84T, A101E, F112Y |
| 68 | P97E, N140H, A149E, K155L |
| 69 | Q102E, N146A, S154E, K160D |
| 70 | Q102E, N214D, Y218E |
| 71 | S144E, A149F, K155L, D197S |
| 72 | S26*, K105N, G141Q |
| 73 | S69V, K86Q, L92E, K192A |
| 74 | V148I, P153D, A172E, K192A |
| 75 | A101E, A172E, N214D, Y218E |
| 76 | A101E, K105N, N214D, Y218E |
| 77 | S69V, Q102E, P153V, T159Q |
| 78 | S69V, Q102E, Q157E, T159Q |

7. The DNase variant of claim 1, wherein the variant has improved storage stability in a liquid detergent compared to a parent DNase without said alterations or to the DNase of SEQ ID NO: 1.

8. The DNase variant of claim 7, wherein the DNase has a residual activity (RA), determined as described in Example 2 herein, of at least 0.2.

9. An isolated polynucleotide encoding the DNase variant of claim 1; a nucleic acid construct or expression vector capable of expressing said polynucleotide; or a host cell comprising said polynucleotide.

10. A method of producing a DNase variant, comprising:
a) cultivating the host cell of claim 9 under conditions suitable for expression of the DNase variant; and
b) recovering the DNase variant.

11. A detergent composition comprising a DNase variant of claim 1 and at least one detergent adjunct ingredient.

12. (canceled)

13. A method for obtaining a DNase variant, comprising
a) introducing into a DNase at least two alterations at positions selected from the group consisting of positions 69, 102, 26, 32, 36, 37, 43, 46, 55, 68, 76, 79, 82, 84, 86, 91, 92, 95, 97, 101, 105, 107, 112, 129, 133, 138, 140, 141, 144, 146, 147, 148, 149, 150, 153, 154, 155, 157, 158, 159, 160, 170, 172, 185, 187, 191, 192, 197, 199, 208, 211, 213, 214, 217 and 218 of SEQ ID NO: 1, wherein the variant has a sequence identity of at least 80%, but less than 100%, to the polypeptide of SEQ ID NO: 1, SEQ ID NO: 2 or SEQ ID NO: 3; and
b) recovering the variant.

14. (canceled)

15. The DNase variant of claim 1, wherein the variant has a sequence identity of at least 90% to the polypeptide of SEQ ID NO: 1, SEQ ID NO: 2 or SEQ ID NO: 3.

16. The DNase variant of claim 1, wherein the variant has a sequence identity of at least 95% to the polypeptide of SEQ ID NO: 1, SEQ ID NO: 2 or SEQ ID NO: 3.

17. The DNase variant of claim 1, wherein the variant has a sequence identity of at least 96% to the polypeptide of SEQ ID NO: 1, SEQ ID NO: 2 or SEQ ID NO: 3.

18. The DNase variant of claim 1, wherein the variant has a sequence identity of at least 97% to the polypeptide of SEQ ID NO: 1, SEQ ID NO: 2 or SEQ ID NO: 3.


SEQUENCE LISTING (3 sequences)

SEQ ID NO: 1 - protein, 221 aa, Aspergillus oryzae
VPVNPEPDATSVENVALKTGSGDSQSDPIKADLEVKGQSALPFDVDCWAILCKGAPNVLQRVNEKTKNSNRDRSGANKGPFKDPQKWGIKALPPKNPSWSAQDFKSPEEYAFASSLQGGTNAILAPVNLASQNSQGGVLNGFYSANKVAQFDPSKPQQTKGTWFQITKFTGAAGPYCKALGSNDKSVCDKNKNIAGDWGFDPAKWAYQYDEKNNKFNYVGK

SEQ ID NO: 2 - protein, 206 aa, Aspergillus oryzae
ALKTGSGDSQSDPIKADLEVKGQSALPFDVDCWAILCKGAPNVLQRVNEKTKNSNRDRSGANKGPFKDPQKWGIKALPPKNPSWSAQDFKSPEEYAFASSLQGGTNAILAPVNLASQNSQGGVLNGFYSANKVAQFDPSKPQQTKGTWFQITKFTGAAGPYCKALGSNDKSVCDKNKNIAGDWGFDPAKWAYQYDEKNNKFNYVGK

SEQ ID NO: 3 - protein, 204 aa, Aspergillus oryzae
KTGSGDSQSDPIKADLEVKGQSALPFDVDCWAILCKGAPNVLQRVNEKTKNSNRDRSGANKGPFKDPQKWGIKALPPKNPSWSAQDFKSPEEYAFASSLQGGTNAILAPVNLASQNSQGGVLNGFYSANKVAQFDPSKPQQTKGTWFQITKFTGAAGPYCKALGSNDKSVCDKNKNIAGDWGFDPAKWAYQYDEKNNKFNYVGK